(19) United States
(12) Patent Application Publication
(10) Pub. No.: US 2022/0242834 A1
(43) Pub. Date: Aug. 4, 2022

(54) Organic Compound, Light-Emitting Device, Light-Emitting Apparatus, Electronic Device, and Lighting Device

(71) Applicant: Semiconductor Energy Laboratory Co., Ltd., Kanagawa-ken (JP)

(72) Inventors: Keito Tosu, Kanagawa (JP); Yuta Kawano, Kanagawa (JP); Airi Ueda, Kanagawa (JP); Takeyoshi Watabe, Kanagawa (JP); Nobuharu Ohsawa, Kanagawa (JP); Stoshi Seo, Kanagawa (JP); Harue Osaka, Kanagawa (JP); Yuko Kubota, Kanagawa (JP)

(73) Assignee: Semiconductor Energy Laboratory Co., Ltd., Kanagawa-ken (JP)

(21) Appl. No.: 17/578,782

(22) Filed: Jan. 19, 2022

(30) Foreign Application Priority Data
Jan. 28, 2021 (JP) 2021-011969

(51) Int. Cl. C07D 251/24; H01L 51/00; H01L 51/50; H01L 51/52

(52) U.S. Cl. CPC C07D 251/24; H01L 51/0067; H01L 2251/552; H01L 51/5072; H01L 51/5016; H01L 51/5237; H01L 51/5004

(57) ABSTRACT

An electron-transport layer material with a low refractive index is provided. An organic compound represented by General Formula (G1) is provided. In General Formula (G1), one to three of Q¹ to Q³ represent N and when one or two of Q¹ to Q³ represent N, the remaining two or one of Q¹ to Q³ represent CH. Furthermore, R⁰ represents any of hydrogen, an alkyl group having 1 to 6 carbon atoms, an alicyclic group having 3 to 10 carbon atoms, and a group represented by Formula (G1-1). At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group.

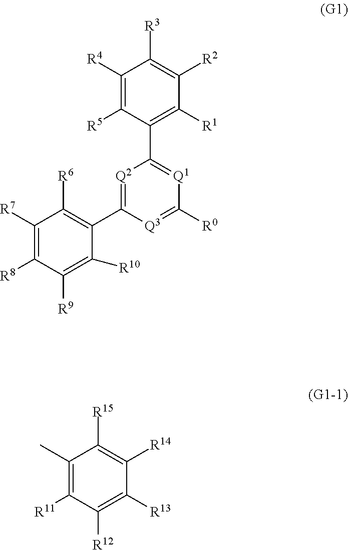

### BACKGROUND OF THE INVENTION

### 1. Field of the Invention

One embodiment of the present invention relates to an organic compound, a light-emitting device, a display module, a lighting module, a display apparatus, a light-emitting apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### 2. Description of the Related Art

Light-emitting devices (organic EL devices) including organic compounds and utilizing electroluminescence (EL) have been put to more practical use. In the basic structure of such light-emitting devices, an organic compound layer containing a light-emitting material (an EL layer) is interposed between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal devices, such as high visibility and no need for backlight when used in pixels of a display, and are suitable as flat panel display devices. Displays including such light-emitting devices are also highly advantageous in that they can be thin and lightweight. Moreover, such light-emitting devices also have a feature that response speed is extremely fast.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, the light-emitting devices also have great potential as planar light sources, which can be used for lighting devices and the like.

Displays or lighting devices including light-emitting devices can be used for a variety of electronic appliances as described above, and research and development of light-emitting devices have progressed for more favorable characteristics.

Low outcoupling efficiency is often a problem in an organic EL device. In particular, the attenuation due to reflection which is caused by a difference in refractive index between adjacent layers is a main cause of a reduction in device efficiency. In order to reduce this effect, a structure including a layer formed using a low refractive index material in an EL layer (see Non-Patent Document 1, for example) has been proposed.

A light-emitting device having this structure can have higher outcoupling efficiency and higher external quantum efficiency than a light-emitting device having a conventional structure; however, it is not easy to form such a layer with a low refractive index in an EL layer without adversely affecting other critical characteristics of the light-emitting device. This is because a low refractive index is in a trade-off relationship with a high carrier-transport property or high reliability of a light-emitting device including a layer with a low refractive index. This problem is caused because the carrier-transport property and reliability of an organic compound largely depend on an unsaturated bond, and an organic compound having many unsaturated bonds tends to have a high refractive index.

### REFERENCE

### Non-Patent Document

[Non-Patent Document 1] Jaeho Lee et al., “Synergetic electrode architecture for efficient graphene-based flexible organic light-emitting diodes”, Nature COMMUNICATIONS, Jun. 2, 2016, DOI: 10.1038/ncomms 11791.

### SUMMARY OF THE INVENTION

An object of one embodiment of the present invention is to provide a novel organic compound. Another object of one embodiment of the present invention is to provide a novel organic compound having a carrier-transport property. Another object of one embodiment of the present invention is to provide a novel organic compound having an electron-transport property. Another object of one embodiment of the present invention is to provide an organic compound with a low refractive index. Another object of one embodiment of the present invention is to provide an organic compound with a low refractive index and a carrier-transport property. Another object of one embodiment of the present invention is to provide an organic compound with a low refractive index and an electron-transport property.

Another object of one embodiment of the present invention is to provide a light-emitting device having high emission efficiency. Another object of one embodiment of the present invention is to provide a light-emitting device having high reliability. Another object of one embodiment of the present invention is to provide a light-emitting device, a light-emitting apparatus, an electronic appliance, a display apparatus, and an electronic device each having low power consumption. Another object of one embodiment of the present invention is to provide a light-emitting device, a light-emitting apparatus, an electronic appliance, a display apparatus, and an electronic device each having low power consumption and high reliability.

Note that the description of these objects does not preclude the existence of other objects. One embodiment of the present invention does not necessarily achieve all the objects listed above. Other objects will be apparent from and can be derived from the description of the specification, the drawings, the claims, and the like.

It is only necessary that at least one of the above-described objects be achieved in the present invention.

One embodiment of the present invention is an organic compound represented by General Formula (G1).

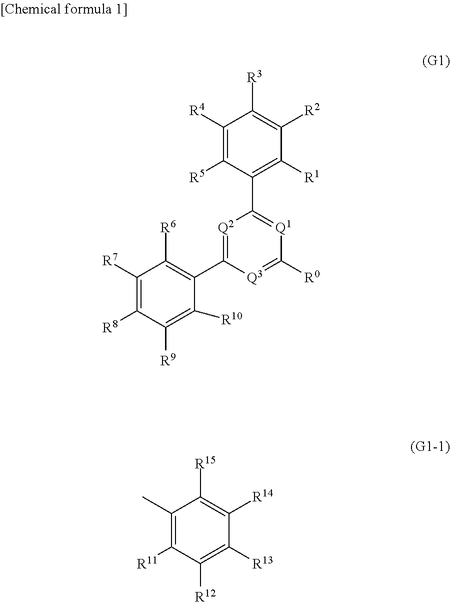

In General Formula (G1), one to three of Q¹ to Q³ represent N and when one or two of Q¹ to Q³ represent N, the remaining two or one of Q¹ to Q³ represent CH. Furthermore, R⁰ represents any of hydrogen, an alkyl group having 1 to 6 carbon atoms, an alicyclic group having 3 to 10 carbon atoms, and a group represented by Formula (G1-1). At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G1) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Another embodiment of the present invention is an organic compound represented by General Formula (G2).

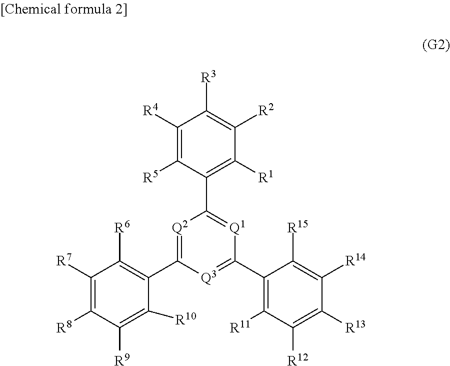

In General Formula (G2), one to three of Q¹ to Q³ represent N and when one or two of Q¹ to Q³ represent N, the remaining two or one of Q¹ to Q³ represent CH. At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G2) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Another embodiment of the present invention is an organic compound represented by General Formula (G3).

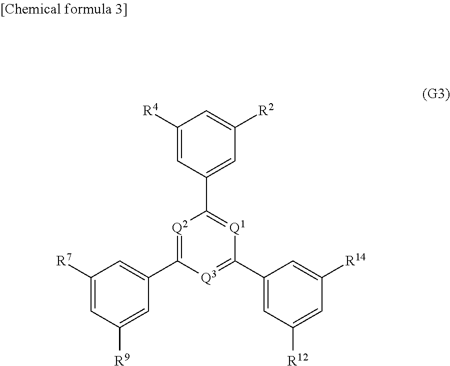

In General Formula (G3), one to three of Q¹ to Q³ represent N and when one or two of Q¹ to Q³ represent N, the remaining two or one of Q¹ to Q³ represent CH. At least one of R², R⁴, R⁷, R⁹, R¹², and R¹⁴ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G3) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

In each of the above structures, the substituted or unsubstituted group including any one of a pyrimidinyl group, a pyrazinyl group, and a triazinyl group in the organic compounds represented by General Formula (G1), General Formula (G2), and General Formula (G3) is preferably represented by Formula (G1-2).

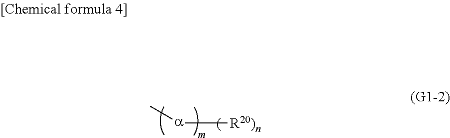

In Formula (G1-2), α represents a substituted or unsubstituted phenylene group. Furthermore, R²⁰ represents any one of a substituted or unsubstituted pyrimidinyl group, a substituted or unsubstituted pyrazinyl group, and a substituted or unsubstituted triazinyl group. Furthermore, m is 0 to 2. In the case where m is 2, a plurality of α's may be the same or different from each other. Furthermore, n is 1 or 2. In the case where n is 2, a plurality of R²⁰'s may be the same or different from each other.

In the above structure, one or both of R² and R⁴ preferably represent the group represented by Formula (G1-2) (note that in the case where both R² and R⁴ represent the groups represented by Formula (G1-2), the two groups represented by Formula (G1-2) may be the same or different from each other).

In each of the above structures, the substituted or unsubstituted group including any one of a pyrimidinyl group, a pyrazinyl group, and a triazinyl group in the organic compounds represented by General Formula (G1), General Formula (G2), and General Formula (G3) is preferably represented by Formula (G1-3).

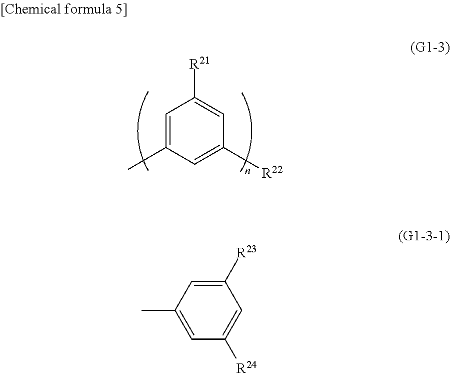

In Formula (G1-3), R²¹ represents any one of hydrogen, an alkyl group having 1 to 6 carbon atoms, an alicyclic group having 3 to 10 carbon atoms, and a group represented by Formula (G1-3-1). In addition, R²² represents the group represented by Formula (G1-3-1). In Formula (G1-3-1), R²³ and R²⁴ each independently represent any one of hydrogen, an alkyl group having 1 to 6 carbon atoms, an alicyclic group having 3 to 10 carbon atoms, a substituted or unsubstituted pyrimidinyl group, a substituted or unsubstituted pyrazinyl group, and a substituted or unsubstituted triazinyl group. At least one of R²³ and R²⁴ represents any one of a substituted or unsubstituted pyrimidinyl group, a substituted or unsubstituted pyrazinyl group, and a substituted or unsubstituted triazinyl group. Furthermore, n is 0 to 2. In the case where n is 2, a plurality of R²¹'s may be the same or different from each other.

In the above structure, one or both of R² and R⁴ preferably represent the group represented by Formula (G1-3) (note that in the case where both R² and R⁴ represent the groups represented by Formula (G1-3), the two groups represented by Formula (G1-3) may be the same or different from each other).

In each of the above structures, in the case where the aromatic hydrocarbon group having 6 to 14 carbon atoms forming a ring in the organic compounds represented by General Formula (G1), General Formula (G2), and General Formula (G3) has a substituent, the substituent is preferably any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded.

In each of the above structures, the aromatic hydrocarbon group having 6 to 14 carbon atoms forming a ring in the organic compounds represented by General Formula (G1), General Formula (G2), and General Formula (G3) is preferably any one of a phenyl group, a naphthyl group, a phenanthrenyl group, and a fluorenyl group.

In each of the above structures, the aromatic hydrocarbon group having 6 to 14 carbon atoms forming a ring in the organic compounds represented by General Formula (G1), General Formula (G2), and General Formula (G3) is preferably represented by any one of Formulae (ra-1) to (ra-16).

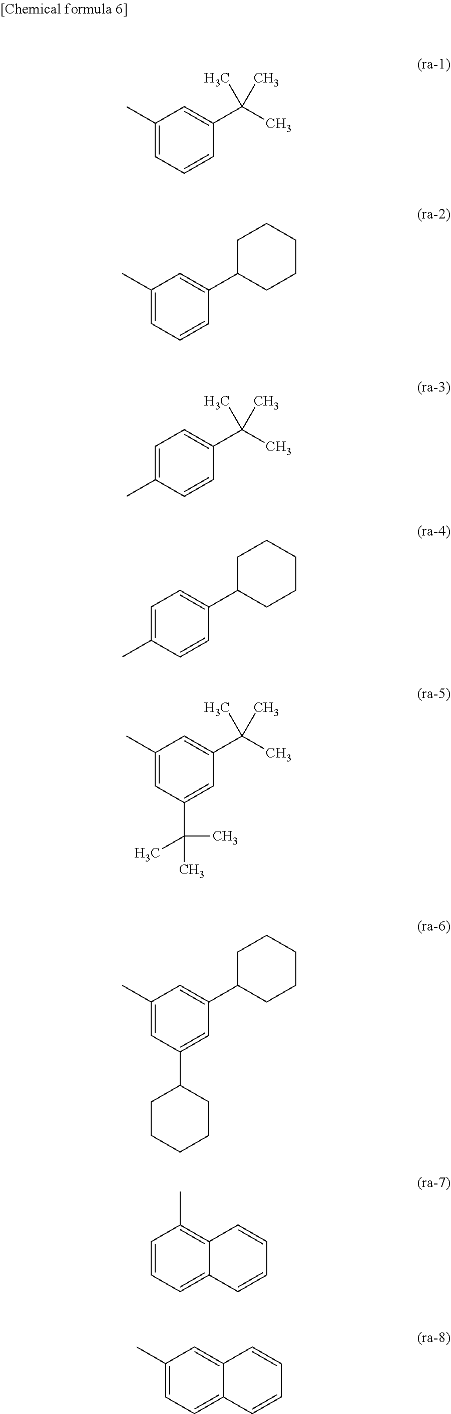

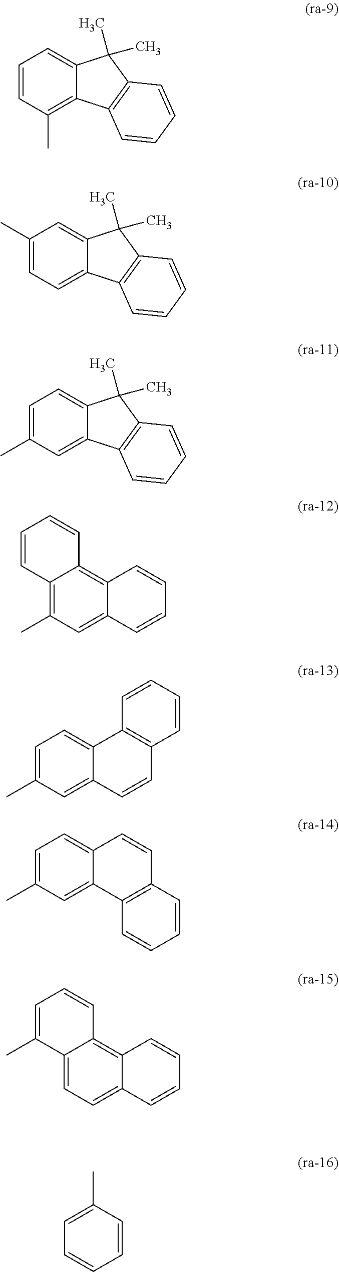

In each of the above structures, the substituted or unsubstituted pyridinyl group in the organic compounds represented by General Formula (G1), General Formula (G2), and General Formula (G3) is preferably an unsubstituted pyridinyl group or a pyridinyl group to which one or more methyl groups are bonded.

In each of the above structures, the alicyclic group is preferably a cycloalkyl group having 3 to 6 carbon atoms in the organic compounds represented by General Formula (G1), General Formula (G2), and General Formula (G3) and the substituted or unsubstituted groups including any one of a pyrimidinyl group, a pyrazinyl group, and a triazinyl group which are represented by Formula (G1-3) and Formula (G1-3-1).

In each of the above structures, the alkyl group having 1 to 6 carbon atoms is preferably a branched alkyl group having 3 to 5 carbon atoms in the organic compounds represented by General Formula (G1), General Formula (G2), and General Formula (G3) and the substituted or unsubstituted groups including any one of a pyrimidinyl group, a pyrazinyl group, and a triazinyl group which are represented by Formula (G1-3) and Formula (G1-3-1).

Another embodiment of the present invention is an organic compound represented by General Formula (G3′).

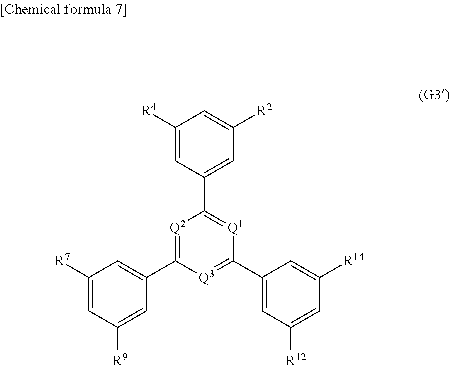

In General Formula (G3′), one to three of Q¹ to Q³ represent N and when one or two of Q¹ to Q³ represent N, the remaining two or one of Q¹ to Q³ represent CH. Furthermore, R² represents a group represented by Formula (R²-1), and R⁴, R⁷, R⁹, R¹², and R¹⁴ each independently represent any one of groups represented by Formulae (r-1) to (r-44). Note that in Formula (R²-1), R represents a substituted or unsubstituted phenylene group or a substituted or unsubstituted biphenyldiyl group, R²⁵ represents any one of the groups represented by Formulae (r-1) to (r-24), and n is 1 or 2. Note that the organic compound represented by General Formula (G3′) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

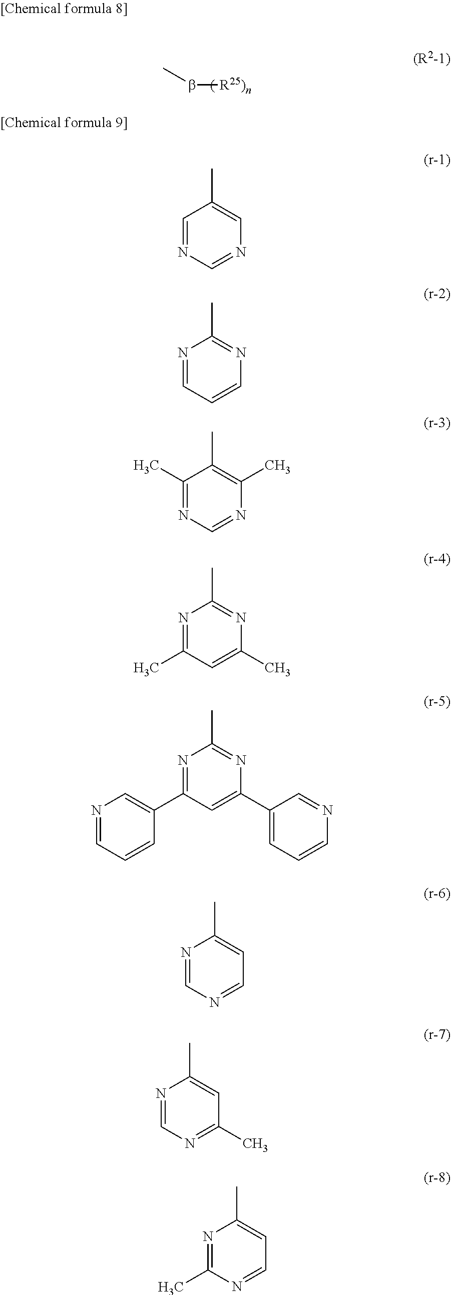

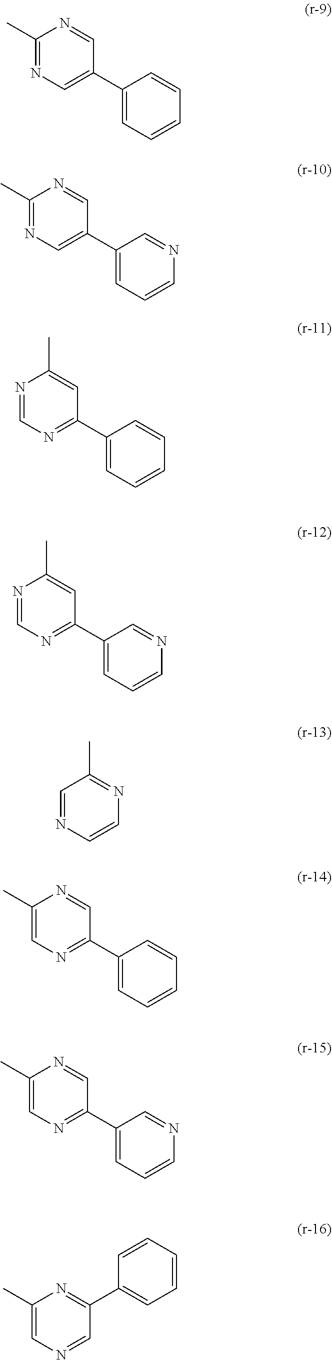

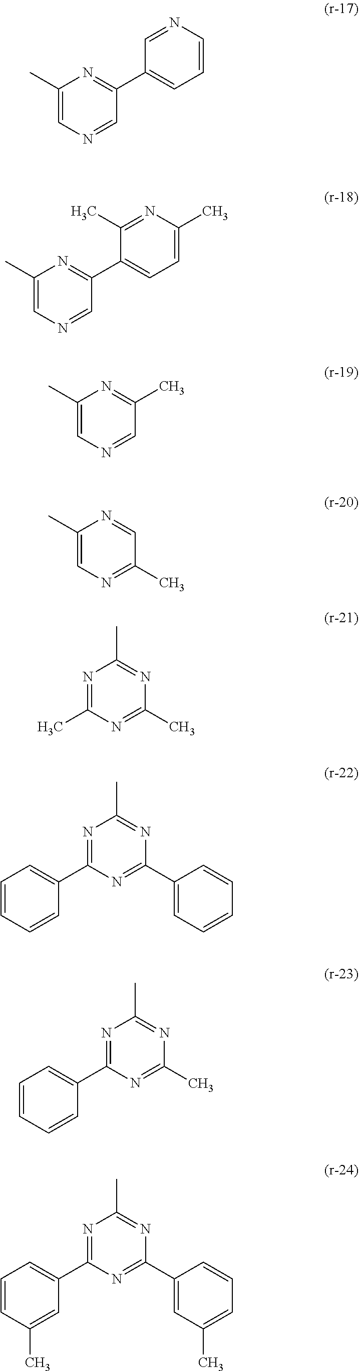

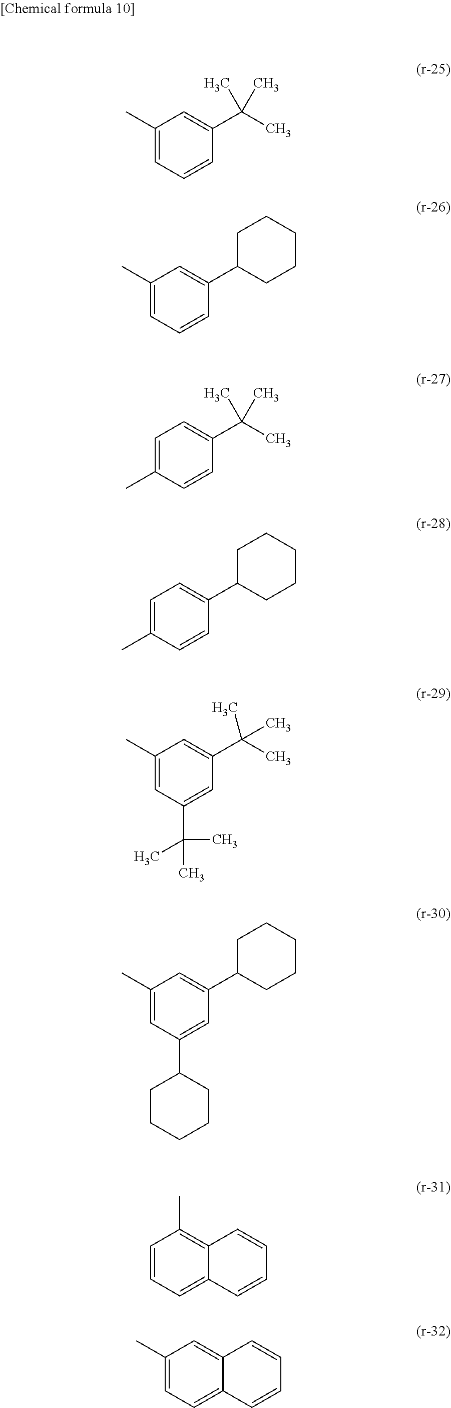

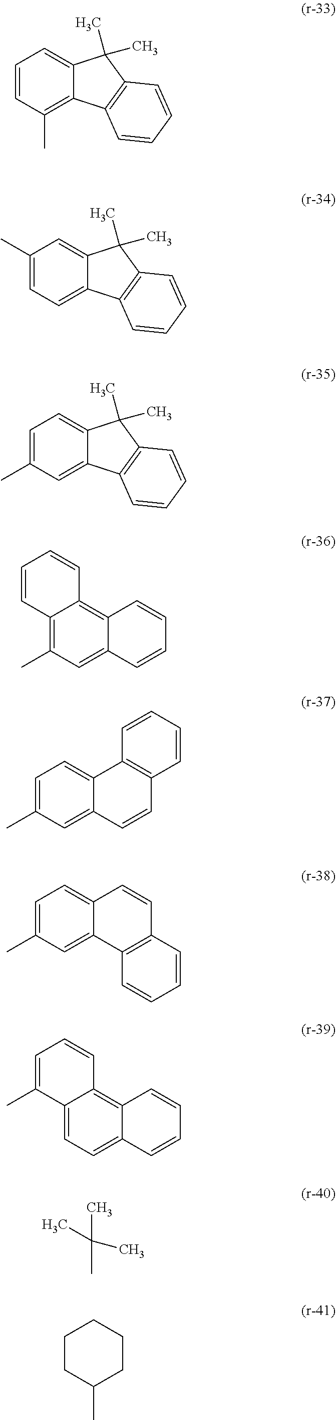

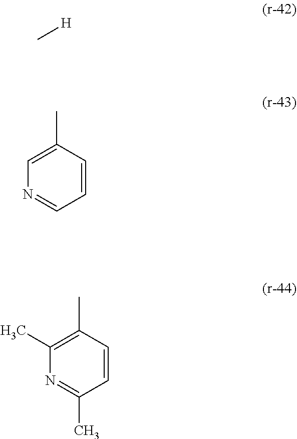

In the above structure, β in the group represented by Formula (R²-1) preferably represents a group represented by any one of Formulae (β-1) to (β-14).

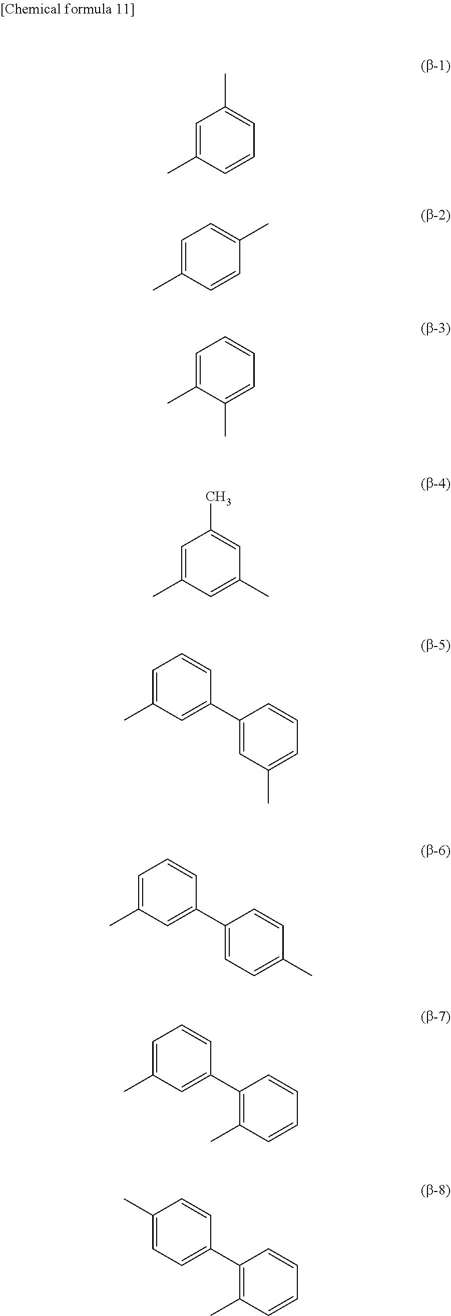

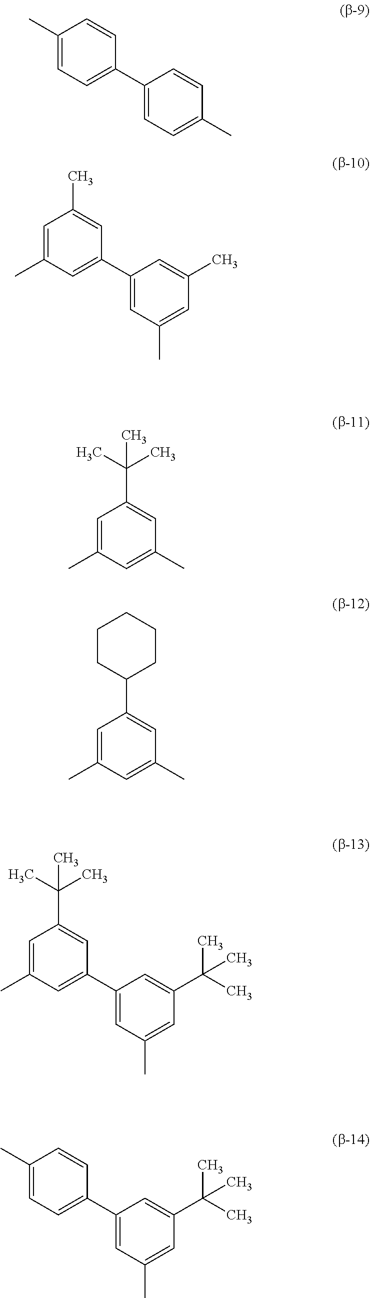

Another embodiment of the present invention is an organic compound represented by Structural Formula (137) or (154).

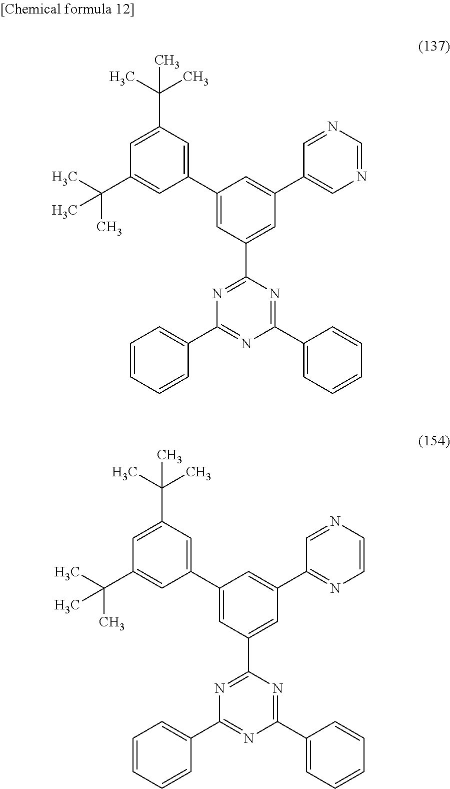 [SMILES: CC(C)(C)c1cc(-c2cc(-c3cncnc3)cc(-c3nc(-c4ccccc4)nc(-c4ccccc4)n3)c2)cc(C(C)(C)C)c1 ; CC(C)(C)c1cc(-c2cc(-c3cnccn3)cc(-c3nc(-c4ccccc4)nc(-c4ccccc4)n3)c2)cc(C(C)(C)C)c1]

Another embodiment of the present invention is a light-emitting device including the above-described organic compound of one embodiment of the present invention. The present invention also includes a light-emitting device including a guest material as well as the above-described organic compound.

Note that the present invention also includes a light-emitting device in which an EL layer provided between a pair of electrodes or a light-emitting layer included in the EL layer contains the organic compound of one embodiment of the present invention. In addition to the aforementioned light-emitting device, the present invention includes a light-emitting device including a layer (e.g., a cap layer) that is in contact with an electrode and contains an organic compound. In addition to the light-emitting devices, a light-emitting apparatus including a transistor, a substrate, and the like is also included in the scope of the invention. Furthermore, an electronic appliance and a lighting device each including any of these light-emitting devices and any of a sensor unit, an input unit, a communication unit, and the like are also included in the scope of the invention.

In addition, the scope of one embodiment of the present invention includes a light-emitting apparatus including a light-emitting device, and a lighting device including the light-emitting apparatus. Accordingly, the light-emitting apparatus in this specification refers to an image display device and a light source (including a lighting device). In addition, the light-emitting apparatus includes the following in its category: a module in which a connector such as a flexible printed circuit (FPC) or a tape carrier package (TCP) is attached to a light-emitting apparatus; a module in which a printed wiring board is provided at the end of a TCP; and a module in which an integrated circuit (IC) is directly mounted on a light-emitting device by a chip on glass (COG) method.

According to one embodiment of the present invention, a novel organic compound can be provided. According to another embodiment of the present invention, a novel organic compound having a carrier-transport property can be provided. According to another embodiment of the present invention, a novel organic compound having an electron-transport property can be provided. According to another embodiment of the present invention, an organic compound with a low refractive index can be provided. According to another embodiment of the present invention, an organic compound with a low refractive index and a carrier-transport property can be provided. According to another embodiment of the present invention, an organic compound with a low refractive index and an electron-transport property can be provided.

According to another embodiment of the present invention, a light-emitting device having high emission efficiency can be provided. According to another embodiment of the present invention, a light-emitting device having high reliability can be provided. According to another embodiment of the present invention, a light-emitting device, a light-emitting apparatus, an electronic appliance, a display apparatus, and an electronic device each having low power consumption can be provided. According to another embodiment of the present invention, a light-emitting device, a light-emitting apparatus, an electronic appliance, a display apparatus, and an electronic device each having low power consumption and high reliability can be provided.

Note that the description of these effects does not preclude the existence of other effects. One embodiment of the present invention does not necessarily have all the effects listed above. Other effects will be apparent from and can be derived from the description of the specification, the drawings, the claims, and the like.

### BRIEF DESCRIPTION OF THE DRAWINGS

In the accompanying drawings:

FIGS. 1A to 1E illustrate structures of light-emitting devices of an embodiment;

FIGS. 2A and 2B illustrate a structure of a light-emitting apparatus of an embodiment;

FIGS. 3A and 3B illustrate a method for manufacturing a light-emitting apparatus of an embodiment;

FIGS. 4A to 4C illustrate a method for manufacturing a light-emitting apparatus of an embodiment;

FIGS. 5A to 5C illustrate a method for manufacturing a light-emitting apparatus of an embodiment;

FIGS. 6A and 6B illustrate a method for manufacturing a light-emitting apparatus of an embodiment;

FIG. 7 illustrates a light-emitting apparatus of an embodiment;

FIGS. 8A and 8B illustrate a light-emitting apparatus of an embodiment;

FIG. 9 illustrates a light-emitting apparatus of an embodiment;

FIGS. 10A to 10C illustrate a method for manufacturing a light-emitting apparatus of an embodiment;

FIGS. 11A and 11B illustrate a method for manufacturing a light-emitting apparatus of an embodiment;

FIG. 12 illustrates a light-emitting apparatus of an embodiment;

FIGS. 13A and 13B illustrate a light-emitting apparatus of an embodiment;

FIGS. 14A and 14B illustrate a light-emitting apparatus of an embodiment;

FIGS. 15A and 15B illustrate light-emitting apparatuses of an embodiment;

FIGS. 16A and 16B illustrate a light-emitting apparatus of an embodiment;

FIGS. 17A to 17E illustrate electronic appliances of an embodiment;

FIGS. 18A to 18E illustrate electronic appliances of an embodiment;

FIGS. 19A and 19B illustrate electronic appliances of an embodiment;

FIGS. 20A and 20B illustrate an electronic appliance of an embodiment;

FIG. 21 illustrates electronic appliances of an embodiment;

FIG. 22 shows an absorption spectrum of mmtBuPh-mPmPTzn;

FIG. 23 shows an MS spectrum of mmtBuPh-mPmPTzn;

FIG. 24 shows measurement data of refractive indices of mmtBuPh-mPmPTzn;

FIG. 25 shows an absorption spectrum of mmtBuPh-mPrPTzn;

FIG. 26 shows an MS spectrum of mmtBuPh-mPrPTzn;

FIG. 27 shows measurement data of refractive indices of mmtBuPh-mPrPTzn;

FIG. 28 shows measurement data of refractive indices of mmtBuPh-mPmPTzn, mPn-mDMePyPTzn, Li-6mq, and Liq;

FIG. 29 shows luminance-current density characteristics of a light-emitting device 1 and a comparative light-emitting device 1;

FIG. 30 shows current efficiency-luminance characteristics of the light-emitting device 1 and the comparative light-emitting device 1;

FIG. 31 shows luminance-voltage characteristics of the light-emitting device 1 and the comparative light-emitting device 1;

FIG. 32 shows current-voltage characteristics of the light-emitting device 1 and the comparative light-emitting device 1;

FIG. 33 shows external quantum efficiency-luminance characteristics of the light-emitting device 1 and the comparative light-emitting device 1;

FIG. 34 shows emission spectra of the light-emitting device 1 and the comparative light-emitting device 1;

FIG. 35 is a graph showing reliabilities of the light-emitting device 1 and the comparative light-emitting device 1;

FIG. 36 shows measurement data of refractive indices of mmtBuPh-mPrPTzn, mPn-mDMePyPTzn, Li-6mq, and Liq;

FIG. 37 shows luminance-current density characteristics of a light-emitting device 2 and a comparative light-emitting device 2;

FIG. 38 shows current efficiency-luminance characteristics of the light-emitting device 2 and the comparative light-emitting device 2;

FIG. 39 shows luminance-voltage characteristics of the light-emitting device 2 and the comparative light-emitting device 2;

FIG. 40 shows current-voltage characteristics of the light-emitting device 2 and the comparative light-emitting device 2;

FIG. 41 shows external quantum efficiency-luminance characteristics of the light-emitting device 2 and the comparative light-emitting device 2;

FIG. 42 shows emission spectra of the light-emitting device 2 and the comparative light-emitting device 2;

FIG. 43 is a graph showing reliabilities of the light-emitting device 2 and the comparative light-emitting device 2;

FIGS. 44A and 44B show ¹H NMR spectra of 2,4mmtBuBP-6PmPPm;

FIGS. 45A and 45B show ¹H NMR spectra of 4mmtBuBP-6PmPPm; and

FIG. 46 shows an absorption spectrum and an emission spectrum of Li-6mq in dehydrated acetone.

### DETAILED DESCRIPTION OF THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

Among organic compounds having a carrier-transport property that can be used for an organic EL device, 1,1-bis[4-[N,N-di(p-tolyl)amino]phenyl]cyclohexane (abbreviation: TAPC) is one of materials with a low refractive index. The use of a material with a low refractive index for an EL layer can increase the external quantum efficiency of a light-emitting device; thus, with TAPC, a light-emitting device with high external quantum efficiency should be obtained. However, TAPC has a heat resistance problem because of its low glass transition temperature. In addition, TAPC can transport holes but cannot transport electrons substantially.

In order to obtain a material with a low refractive index, an atom with low atomic refraction or a substituent with low molecular refraction is preferably introduced into the molecule. Examples of the substituent with low molecular refraction include a saturated hydrocarbon group and a cyclic saturated hydrocarbon group.

In general, a carrier-transport property and a refractive index have a trade-off relationship; an increase in a carrier-transport property causes an increase in a refractive index. This is because the carrier-transport property of an organic compound largely depends on an unsaturated bond, and an organic compound having many unsaturated bonds tends to have a high refractive index.

As is generally known, an electron-transport organic compound is required to have sufficient mobility, stability, and the like when used in an organic EL device but inherently cannot be easily imparted with such properties as compared with a hole-transport organic compound because the required lowest unoccupied molecular orbital (LUMO) level is low. Thus, introduction of a saturated hydrocarbon group, which adversely affects those characteristics, has been considered undesirable.

Contrary to the commonly accepted theory, the present inventors have developed, as a compound having both a carrier-transport property and a low refractive index, a light-emitting device material containing an organic compound which has a pyrimidine skeleton, a pyrazine skeleton, a diazine skeleton, or a triazine skeleton and in which the proportion of carbon atoms forming bonds by the sp³ hybrid orbitals, which form a saturated hydrocarbon group, is within a certain range. Since the light-emitting device material has both the electron-transport property and the optical characteristics such as a low refractive index, the light-emitting device material is suitable for electron-transport layers of photoelectronics devices such as a light-emitting device and a photoelectric conversion device, and can also be used as an electron-transport layer material. The organic compound contained in the light-emitting device material and the electron-transport layer material can achieve a low refractive index while maintaining a high electron-transport property when the number of substituents containing carbon atoms forming bonds by the sp³ hybrid orbitals or the site of substitution in the organic compound is adjusted. When the proportion of carbon atoms forming bonds by the sp³ hybrid orbitals in the organic compound is within a certain range, a light-emitting device material and an electron-transport layer material each having not only a low refractive index and a high electron-transport property but also heat resistance such as a high glass transition temperature can be obtained.

The use of the above light-emitting device material for an EL layer of a light-emitting device can increase the outcoupling efficiency of the EL layer because of the low refractive index, which can improve the emission efficiency of the light-emitting device.

The above electron-transport layer material has a high electron-transport property and thus is suitable for an electron-transport layer of an EL layer in a light-emitting device. In addition, the electron-transport layer material can increase the outcoupling efficiency of the EL layer because of the low refractive index, which can improve the emission efficiency of the light-emitting device. Moreover, the electron-transport layer material of one embodiment of the present invention has a high electron-transport property and a property of transmitting light (in particular, visible light) and thus is suitable for an electron-transport layer of a photoelectric conversion device.

The light-emitting device material or the electron-transport layer material contains an organic compound including at least one six-membered heteroaromatic ring having 1 to 3 nitrogen atoms. The glass transition temperature of the organic compound is higher than or equal to 90° C. The refractive index of a layer containing the organic compound is higher than or equal to 1.5 and lower than or equal to 1.75. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. The proportion of carbon atoms forming bonds by the sp³ hybrid orbitals in the total number of carbon atoms in a molecule of the organic compound is higher than or equal to 10% and lower than or equal to 60%. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. In the results of ¹H-NMR measurement conducted on the organic compound, the integral value of signals at lower than 4 ppm is equal to or more than half the integral value of signals at higher than or equal to 4 ppm.

Note that the heteroaromatic ring in the organic compound is preferably a triazine ring or a diazine ring, further preferably a triazine ring or a pyrimidine ring. The glass transition temperature is preferably higher than or equal to 100° C., further preferably higher than or equal to 110° C., still further preferably higher than or equal to 120° C.

The above light-emitting device material or electron-transport layer material contains an organic compound including at least one six-membered heteroaromatic ring having 1 to 3 nitrogen atoms and a plurality of aromatic hydrocarbon rings each having 6 to 14 carbon atoms forming a ring. At least two of the plurality of aromatic hydrocarbon rings are benzene rings. The organic compound has a plurality of hydrocarbon groups forming bonds by the sp³ hybrid orbitals. The ordinary refractive index of a layer containing the organic compound with respect to light with a wavelength in the range of 455 nm to 465 nm is higher than or equal to 1.5 and lower than or equal to 1.75. The benzene rings are each preferably a monocyclic benzene ring, i.e., a benzene ring to which no aromatic ring is fused.

Note that the proportion of carbon atoms forming bonds by the sp³ hybrid orbitals in the above structure affects the refractive index of the organic compound. That is, an increase in the number of carbon atoms forming bonds by the sp³ hybrid orbitals lowers the refractive index; thus, the outcoupling efficiency of a light-emitting device containing the above-described material can be improved.

The above electron-transport layer material contains an organic compound including at least one six-membered heteroaromatic ring having 1 to 3 nitrogen atoms and a plurality of aromatic hydrocarbon rings each having 6 to 14 carbon atoms forming a ring. At least two of the plurality of aromatic hydrocarbon rings are benzene rings. The organic compound has a plurality of hydrocarbon groups forming bonds by the sp³ hybrid orbitals. The proportion of carbon atoms forming bonds by the sp³ hybrid orbitals in the total number of carbon atoms in a molecule of the organic compound is preferably higher than or equal to 10% and lower than or equal to 60%.

Note that the proportion of carbon atoms forming bonds by the sp³ hybrid orbitals in the above structure affects the refractive index of the organic compound. That is, an increase in the number of carbon atoms forming bonds by the sp³ hybrid orbitals lowers the refractive index; thus, the outcoupling efficiency of a light-emitting device containing the above-described material can be improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, the proportion of carbon atoms forming bonds by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming bonds by the sp³ hybrid orbitals in the total number of carbon atoms in a molecule of the organic compound is preferably higher than or equal to 20% and lower than or equal to 40%.

The above electron-transport layer material contains an organic compound including at least one six-membered heteroaromatic ring having 1 to 3 nitrogen atoms and a plurality of aromatic hydrocarbon rings each having 6 to 14 carbon atoms forming a ring. At least two of the plurality of aromatic hydrocarbon rings are benzene rings. The organic compound has a plurality of hydrocarbon groups forming bonds by the sp³ hybrid orbitals. In the results of ¹H-NMR measurement conducted on the organic compound, the integral value of signals at lower than 4 ppm is preferably equal to or more than half the integral value of signals at higher than or equal to 4 ppm.

Note that the proportion of carbon atoms forming bonds by the sp³ hybrid orbitals in the above structure affects the refractive index of the organic compound. That is, an increase in the number of carbon atoms forming bonds by the sp³ hybrid orbitals lowers the refractive index; thus, the outcoupling efficiency of a light-emitting device containing the above-described material can be improved. Furthermore, the number of carbon atoms forming bonds by the sp³ hybrid orbitals is preferably large, in which case the index of heat resistance such as a glass transition temperature is improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, in the results of ¹H-NMR measurement conducted on the organic compound, the integral value of signals at lower than 4 ppm derived from protons of an alkyl group and an alicyclic group is preferably more than or equal to half and less than or equal to twice, further preferably more than or equal to one times and less than or equal to one and a half times the integral value of signals at higher than or equal to 4 ppm derived from an aryl group or a heteroaromatic group.

The molecular weight of the organic compound contained in the light-emitting device material or the electron-transport layer material is preferably greater than or equal to 500 and less than or equal to 2000. The molecular weight is further preferably greater than or equal to 700 and less than or equal to 1500, in which case the thermophysical property (glass transition temperature) is high and decomposition is unlikely to occur at the time of sublimation (vapor deposition).

In the molecule of the organic compound contained in the light-emitting device material or the electron-transport layer material, it is preferable that the hydrocarbon groups forming bonds by the sp³ hybrid orbitals be each bonded to the aromatic hydrocarbon rings over which the LUMO is not distributed, i.e., the LUMO be distributed over a ring other than the aromatic hydrocarbon rings to which the hydrocarbon groups are bonded in the molecule of the organic compound. Note that in this specification, the expression “the LUMO is not distributed over the aromatic hydrocarbon rings to which the hydrocarbon groups are bonded” means that the isovalue of the LUMO distribution density in the aromatic hydrocarbon rings to which the hydrocarbon groups are bonded is less than 0.06 [electrons/au³], preferably less than 0.02 [electrons/au³].

It is further preferable that the LUMO be mainly distributed over the heteroaromatic ring and a substituent directly bonded to the heteroaromatic ring. When the LUMO is distributed in the above manner in the molecule, an overlap of the LUMO between the organic compound molecules which are close to each other in the solid (film) state is likely to occur and thus transportation of electrons is facilitated, which can decrease the driving voltage.

Note that the isovalue of the LUMO can be obtained through molecular orbital calculation with Gaussian, for example.

In the molecule of the organic compound contained in the light-emitting device material or the electron-transport layer material, it is preferable that at least one of the aromatic hydrocarbon rings to which the hydrocarbon groups forming bonds by the sp³ hybrid orbitals are bonded be a benzene ring.

It is preferable that the organic compound contained in the light-emitting device material or the electron-transport layer material include at least three benzene rings, the three benzene rings be each bonded to the six-membered heteroaromatic ring, and two of the three benzene rings be each a substituted or unsubstituted phenyl group and include no hydrocarbon group. The six-membered heteroaromatic ring is preferably a triazine ring or a pyrimidine ring.

The organic compound contained in the light-emitting device material or the electron-transport layer material preferably includes a substituted or unsubstituted pyridinyl group, in which case the property of electron injection from a cathode or an electron-injection layer can be increased.

It is preferable that the hydrocarbon groups forming bonds by the sp³ hybrid orbitals in the organic compound contained in the light-emitting device material or the electron-transport layer material be each an alkyl group or a cycloalkyl group, and the alkyl group be a branched alkyl group having 3 to 5 carbon atoms.

Note that in the light-emitting device material or the electron-transport layer material, the glass transition temperature of the organic compound is preferably higher than or equal to 90° C. The glass transition temperature is further preferably higher than or equal to 100° C., still further preferably higher than or equal to 110° C., particularly preferably higher than or equal to 120° C.

Next, organic compounds of embodiments of the present invention, each of which can be used as one mode of the organic compound contained in the light-emitting device material or the electron-transport layer material, will be described below.

That is, one embodiment of the present invention is an organic compound represented by General Formula (G1).

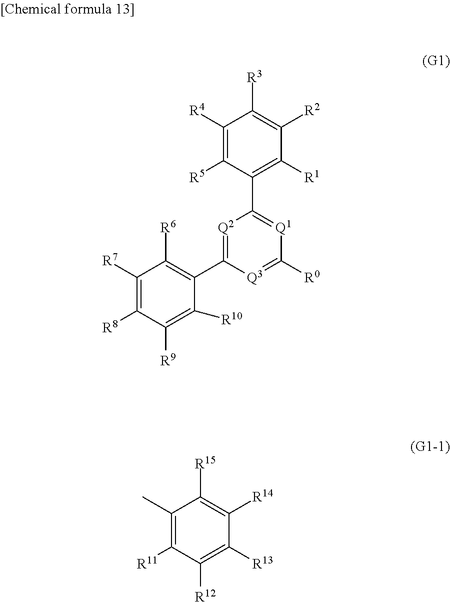

In General Formula (G1), one to three of Q¹ to Q³ represent N and when one or two of Q¹ to Q³ represent N, the remaining two or one of Q¹ to Q³ represent CH. Furthermore, R⁰ represents any of hydrogen, an alkyl group having 1 to 6 carbon atoms, an alicyclic group having 3 to 10 carbon atoms, and a group represented by Formula (G1-1). At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G1) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Note that in the structure of the organic compound represented by General Formula (G1), the proportion of carbon atoms forming bonds by the sp³ hybrid orbitals affects the refractive index of the organic compound. That is, an increase in the number of carbon atoms forming bonds by the sp³ hybrid orbitals lowers the refractive index; thus, the outcoupling efficiency of a light-emitting device including the organic compound can be improved. Furthermore, the number of carbon atoms forming bonds by the sp³ hybrid orbitals is preferably large, in which case the index of heat resistance such as a glass transition temperature is improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, the proportion of carbon atoms forming bonds by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming bonds by the sp³ hybrid orbitals in the total number of carbon atoms in a molecule of the organic compound is preferably higher than or equal to 20% and lower than or equal to 40%.

The organic compound represented by General Formula (G1) is preferably formed of only a six-membered heteroaromatic ring/six-membered heteroaromatic rings having 1 to 3 nitrogen atoms, a six-membered aromatic ring/six-membered aromatic rings (i.e., a substituted or unsubstituted phenyl group), and a hydrocarbon group/hydrocarbon groups forming bonds by the sp³ hybrid orbitals (e.g., an alkyl group or an alicyclic group), that is, the organic compound preferably includes no fused ring, in which case the refractive index is lowered and the transport property of carriers (electrons) is increased.

A pyridine ring, a pyrimidine ring, or a triazine ring can be used as the six-membered ring including Q¹ to Q³ in the organic compound represented by General Formula (G1). In the case where the organic compound represented by General Formula (G1) is used for a layer in contact with a light-emitting layer or a layer in contact with an active layer, any of a triazine ring, a pyrazine ring, and a pyrimidine ring, which easily inject electrons into these layers and have a high electron-transport property, is preferably used, and a triazine ring is particularly preferable.

The total number of substituents (an alkyl group and an alicyclic group) per molecule in the organic compound represented by General Formula (G1) is preferably greater than or equal to 4 and less than or equal to 10 in consideration of the synthesis cost, further preferably greater than or equal to 6 in order to lower the refractive index. Similarly, using as large substituents (e.g., an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, a substituted or unsubstituted naphthyl group, a substituted or unsubstituted phenanthryl group, or a substituted or unsubstituted fluorenyl group can be used as the aromatic hydrocarbon group having 6 to 14 carbon atoms forming a ring in the organic compound represented by General Formula (G1). It is particularly preferable to use the phenyl group to reduce the refractive index. It is preferable to use the naphthyl group, the phenanthryl group, or the fluorenyl group to increase the glass transition temperature. A branched alkyl or cycloalkyl group having 3 to 5 carbon atoms is preferably bonded to the aromatic hydrocarbon group having 6 to 14 carbon atoms forming a ring, in which case effects of increasing the glass transition temperature and suppressing an increase in the refractive index, i.e., maintaining a low refractive index, can be produced. In order to lower the refractive index, any of an alkyl group having 1 to 6 carbon atoms, an alicyclic group having 3 to 10 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded is preferably bonded to the aromatic hydrocarbon group having 6 to 14 carbon atoms forming a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a fused ring is used and the number of fused rings is three or more, one six-membered ring is preferably fused with the other six-membered rings only at the a-face and at least one of the c-face and the e-face, in which case the refractive index can be lowered as compared with the case of polyacene. For example, the refractive index of the case of using a phenanthrene ring can be lower than that of the case of using an anthracene ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G1). As the alicyclic group having 3 to 10 carbon atoms, a cyclopropyl group, a cyclohexyl group, a cyclodecanyl group, a bicyclooctyl group, an adamantyl group, or the like can be used.

Note that some or all of the hydrogen atoms in the organic compound represented by General Formula (G1) can be deuterium atoms. In this case, the use of the organic compound in a light-emitting layer, a layer in contact with the light-emitting layer, or the like in a light-emitting device is expected to enable the device to have a long lifetime. The organic compound in which all the hydrogen atoms are protium is also preferable because its synthesis cost can be lower.

Another embodiment of the present invention is an organic compound represented by General Formula (G2).

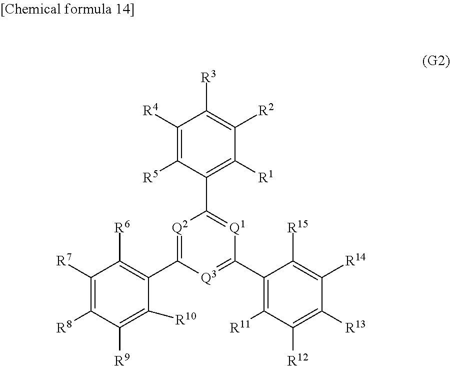

In General Formula (G2), one to three of Q¹ to Q³ represent N and when one or two of Q¹ to Q³ represent N, the remaining two or one of Q¹ to Q³ represent CH. At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G2) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Note that in the structure of the organic compound represented by General Formula (G2), the proportion of carbon atoms forming bonds by the sp³ hybrid orbitals affects the refractive index of the organic compound. That is, an increase in the number of carbon atoms forming bonds by the sp³ hybrid orbitals lowers the refractive index; thus, the outcoupling efficiency of a light-emitting device including the organic compound can be improved. Furthermore, the number of carbon atoms forming bonds by the sp³ hybrid orbitals is preferably large, in which case the index of heat resistance such as a glass transition temperature is improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, the proportion of carbon atoms forming bonds by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming bonds by the sp³ hybrid orbitals in the total number of carbon atoms in a molecule of the organic compound is preferably higher than or equal to 20% and lower than or equal to 40%.

The organic compound represented by General Formula (G2) is preferably formed of only a six-membered heteroaromatic ring/six-membered heteroaromatic rings including Q¹ to Q³, a six-membered aromatic ring/six-membered aromatic rings (i.e., a substituted or unsubstituted phenyl group), and a hydrocarbon group/hydrocarbon groups forming bonds by the sp³ hybrid orbitals (e.g., an alkyl group or an alicyclic group), that is, the organic compound preferably includes no fused ring, in which case the refractive index is lowered and the transport property of carriers (electrons) is increased.

A pyridine ring, a pyrimidine ring, or a triazine ring can be used as the six-membered ring including Q¹ to Q³ in the organic compound represented by General Formula (G2). In the case where the organic compound represented by General Formula (G2) is used for a layer in contact with a light-emitting layer or a layer in contact with an active layer, any of a triazine ring, a pyrazine ring, and a pyrimidine ring, which easily inject electrons into these layers and have a high electron-transport property, is preferably used, and a triazine ring is particularly preferable.

The total number of substituents (an alkyl group and an alicyclic group) per molecule in the organic compound represented by General Formula (G2) is preferably greater than or equal to 4 and less than or equal to 10 in consideration of the synthesis cost, further preferably greater than or equal to 6 in order to lower the refractive index. Similarly, using as large substituents (e.g., an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, a substituted or unsubstituted naphthyl group, a substituted or unsubstituted phenanthryl group, or a substituted or unsubstituted fluorenyl group can be used as the aromatic hydrocarbon group having 6 to 14 carbon atoms forming a ring in the organic compound represented by General Formula (G2). It is particularly preferable to use the phenyl group to reduce the refractive index. It is preferable to use the naphthyl group, the phenanthryl group, or the fluorenyl group to increase the glass transition temperature. A branched alkyl or cycloalkyl group having 3 to 5 carbon atoms is preferably bonded to the aromatic hydrocarbon group having 6 to 14 carbon atoms forming a ring, in which case effects of increasing the glass transition temperature and suppressing an increase in the refractive index, i.e., maintaining a low refractive index, can be produced. In order to lower the refractive index, any of an alkyl group having 1 to 6 carbon atoms, an alicyclic group having 3 to 10 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded is preferably bonded to the aromatic hydrocarbon group having 6 to 14 carbon atoms forming a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a fused ring is used and the number of fused rings is three or more, one six-membered ring is preferably fused with the other six-membered rings only at the a-face and at least one of the c-face and the e-face, in which case the refractive index can be lowered as compared with the case of polyacene. For example, the refractive index of the case of using a phenanthrene ring can be lower than that of the case of using an anthracene ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G2). As the alicyclic group having 3 to 10 carbon atoms, a cyclopropyl group, a cyclohexyl group, a cyclodecanyl group, a bicyclooctyl group, an adamantyl group, or the like can be used.

Note that some or all of the hydrogen atoms in the organic compound represented by General Formula (G2) can be deuterium atoms. In this case, the use of the organic compound in a light-emitting layer, a layer in contact with the light-emitting layer, or the like in a light-emitting device is expected to enable the device to have a long lifetime. The organic compound in which all the hydrogen atoms are protium is also preferable because its synthesis cost can be lower.

Another embodiment of the present invention is an organic compound represented by General Formula (G3).

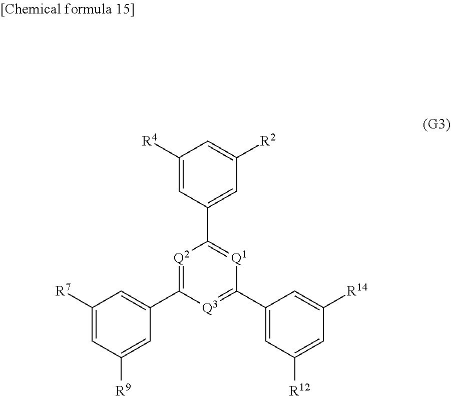

In General Formula (G3), one to three of Q¹ to Q³ represent N and when one or two of Q¹ to Q³ represent N, the remaining two or one of Q¹ to Q³ represent CH. At least one of R², R⁴, R⁷, R⁹, R¹², and R¹⁴ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G3) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Note that in the structure of the organic compound represented by General Formula (G3), the proportion of carbon atoms forming bonds by the sp³ hybrid orbitals affects the refractive index of the organic compound. That is, an increase in the number of carbon atoms forming bonds by the sp³ hybrid orbitals lowers the refractive index; thus, the outcoupling efficiency of a light-emitting device including the organic compound can be improved. Furthermore, the number of carbon atoms forming bonds by the sp³ hybrid orbitals is preferably large, in which case the index of heat resistance such as a glass transition temperature is improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, the proportion of carbon atoms forming bonds by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming bonds by the sp³ hybrid orbitals in the total number of carbon atoms in a molecule of the organic compound is preferably higher than or equal to 20% and lower than or equal to 40%.

A pyridine ring, a pyrimidine ring, or a triazine ring can be used as the six-membered ring including Q¹ to Q³ in the organic compound represented by General Formula (G3). In the case where the organic compound represented by General Formula (G3) is used for a layer in contact with a light-emitting layer or a layer in contact with an active layer, any of a triazine ring, a pyrazine ring, and a pyrimidine ring, which easily inject electrons into these layers and have a high electron-transport property, is preferably used, and a triazine ring is particularly preferable.

The total number of substituents (an alkyl group and an alicyclic group) per molecule in the organic compound represented by General Formula (G3) is preferably greater than or equal to 4 and less than or equal to 10 in consideration of the synthesis cost, further preferably greater than or equal to 6 in order to lower the refractive index. Similarly, using as large substituents (e.g., an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, a substituted or unsubstituted naphthyl group, a substituted or unsubstituted phenanthryl group, or a substituted or unsubstituted fluorenyl group can be used as the aromatic hydrocarbon group having 6 to 14 carbon atoms forming a ring in the organic compound represented by General Formula (G3). It is particularly preferable to use the phenyl group to reduce the refractive index. It is preferable to use the naphthyl group, the phenanthryl group, or the fluorenyl group to increase the glass transition temperature. A branched alkyl or cycloalkyl group having 3 to 5 carbon atoms is preferably bonded to the aromatic hydrocarbon group having 6 to 14 carbon atoms forming a ring, in which case effects of increasing the glass transition temperature and suppressing an increase in the refractive index, i.e., maintaining a low refractive index, can be produced. In order to lower the refractive index, any of an alkyl group having 1 to 6 carbon atoms, an alicyclic group having 3 to 10 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded is preferably bonded to the aromatic hydrocarbon group having 6 to 14 carbon atoms forming a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a fused ring is used and the number of fused rings is three or more, one six-membered ring is preferably fused with the other six-membered rings only at the a-face and at least one of the c-face and the e-face, in which case the refractive index can be lowered as compared with the case of polyacene. For example, the refractive index of the case of using a phenanthrene ring can be lower than that of the case of using an anthracene ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G3). As the alicyclic group having 3 to 10 carbon atoms, a cyclopropyl group, a cyclohexyl group, a cyclodecanyl group, a bicyclooctyl group, an adamantyl group, or the like can be used.

Note that some or all of the hydrogen atoms in the organic compound represented by General Formula (G3) can be deuterium atoms. In this case, the use of the organic compound in a light-emitting layer, a layer in contact with the light-emitting layer, or the like in a light-emitting device is expected to enable the device to have a long lifetime. The organic compound in which all the hydrogen atoms are protium is also preferable because its synthesis cost can be lower.

In each of the above structures, the substituted or unsubstituted group including any one of a pyrimidinyl group, a pyrazinyl group, and a triazinyl group in the organic compounds represented by General Formula (G1), General Formula (G2), and General Formula (G3) is preferably represented by Formula (G1-2). Any of the organic compounds which includes the group represented by Formula (G1-2) is preferably used in an electron-transport layer of a light-emitting device, in which case electron injection from the cathode side is enhanced and the driving voltage can be reduced. In this case, it is preferable to use a mixed layer of the organic compound represented by any of General Formula (G1), General Formula (G2), and General Formula (G3) and an alkyl complex such as 6-methyl-8-quinolinolato-lithium (abbreviation: Li-6mq) to further reduce the driving voltage and increase the outcoupling efficiency and emission efficiency.

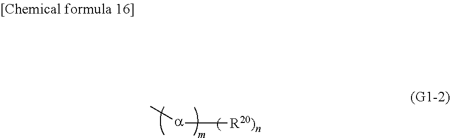

In Formula (G1-2), α represents a substituted or unsubstituted phenylene group. Furthermore, R²⁰ represents any one of a substituted or unsubstituted pyrimidinyl group, a substituted or unsubstituted pyrazinyl group, and a substituted or unsubstituted triazinyl group. Furthermore, m is 0 to 2. In the case where m is 2, a plurality of α's may be the same or different from each other. Furthermore, n is 1 or 2. In the case where n is 2, a plurality of R²⁰'s may be the same or different from each other.

In each of the above structures, one or both of R² and R⁴ in the organic compounds represented by General Formula (G1), General Formula (G2), and General Formula (G3) preferably represent the substituted or unsubstituted group including any one of a pyrimidinyl group, a pyrazinyl group, and a triazinyl group which is represented by Formula (G1-2). Note that in the case where both R² and R⁴ represent the groups represented by Formula (G1-2), the two groups represented by Formula (G1-2) may be the same or different from each other.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the substituted or unsubstituted group including any one of a pyrimidinyl group, a pyrazinyl group, and a triazinyl group which is represented by Formula (G1-2). As the alicyclic group having 3 to 10 carbon atoms, a cyclopropyl group, a cyclohexyl group, a cyclodecanyl group, a bicyclooctyl group, an adamantyl group, or the like can be used.

In each of the above structures, the substituted or unsubstituted group including any one of a pyrimidinyl group, a pyrazinyl group, and a triazinyl group in the organic compounds represented by General Formula (G1), General Formula (G2), and General Formula (G3) is preferably represented by Formula (G1-3).

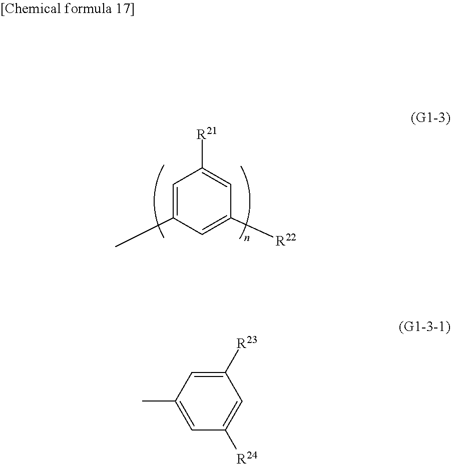

In Formula (G1-3), R²¹ represents any one of hydrogen, an alkyl group having 1 to 6 carbon atoms, an alicyclic group having 3 to 10 carbon atoms, and a group represented by Formula (G1-3-1). In addition, R²² represents the group represented by Formula (G1-3-1). In Formula (G1-3-1), R²³ and R²⁴ each independently represent any one of hydrogen, an alkyl group having 1 to 6 carbon atoms, an alicyclic group having 3 to 10 carbon atoms, a substituted or unsubstituted pyrimidinyl group, a substituted or unsubstituted pyrazinyl group, and a substituted or unsubstituted triazinyl group. At least one of R²³ and R²⁴ represents any one of a substituted or unsubstituted pyrimidinyl group, a substituted or unsubstituted pyrazinyl group, and a substituted or unsubstituted triazinyl group. Furthermore, n is 0 to 2. In the case where n is 2, a plurality of R²¹'s may be the same or different from each other.

Note that one or both of R² and R⁴ in the organic compounds represented by General Formula (G1), General Formula (G2), and General Formula (G3) preferably represent the substituted or unsubstituted group including any one of a pyrimidinyl group, a pyrazinyl group, and a triazinyl group which is represented by Formula (G1-3). Note that in the case where both R² and R⁴ represent the groups represented by Formula (G1-3), the two groups represented by Formula (G1-3) may be the same or different from each other.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the substituted or unsubstituted group including any one of a pyrimidinyl group, a pyrazinyl group, and a triazinyl group which is represented by Formula (G1-3). As the alicyclic group having 3 to 10 carbon atoms, a cyclopropyl group, a cyclohexyl group, a cyclodecanyl group, a bicyclooctyl group, an adamantyl group, or the like can be used.

In each of the above structures, in the case where the aromatic hydrocarbon group having 6 to 14 carbon atoms forming a ring in the organic compounds represented by General Formula (G1), General Formula (G2), and General Formula (G3) has a substituent, the substituent is preferably any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms. As the alicyclic group having 3 to 10 carbon atoms, a cyclopropyl group, a cyclohexyl group, a cyclodecanyl group, a bicyclooctyl group, an adamantyl group, or the like can be used.

In each of the above structures, the aromatic hydrocarbon group having 6 to 14 carbon atoms forming a ring in the organic compounds represented by General Formula (G1), General Formula (G2), and General Formula (G3) is preferably any one of a phenyl group, a naphthyl group, a phenanthrenyl group, and a fluorenyl group.

It is particularly preferable to use the phenyl group to reduce the refractive index. It is preferable to use the naphthyl group, the phenanthryl group, or the fluorenyl group to increase the glass transition temperature. A branched alkyl or cycloalkyl group having 3 to 5 carbon atoms is preferably bonded to the aromatic hydrocarbon group having 6 to 14 carbon atoms forming a ring, in which case effects of increasing the glass transition temperature and suppressing an increase in the refractive index, i.e., maintaining a low refractive index, can be produced. In order to lower the refractive index, any of an alkyl group having 1 to 6 carbon atoms, an alicyclic group having 3 to 10 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded is preferably bonded to the aromatic hydrocarbon group having 6 to 14 carbon atoms forming a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a fused ring is used and the number of fused rings is three or more, one six-membered ring is preferably fused with the other six-membered rings only at the a-face and at least one of the c-face and the e-face, in which case the refractive index can be lowered as compared with the case of polyacene. For example, the refractive index of the case of using a phenanthrene ring can be lower than that of the case of using an anthracene ring.

In each of the above structures, the aromatic hydrocarbon group having 6 to 14 carbon atoms forming a ring in the organic compounds represented by General Formula (G1), General Formula (G2), and General Formula (G3) is preferably represented by any one of Formulae (ra-1) to (ra-16). It is particularly preferable to use a group including a cyclohexyl group, examples of which are represented by Formulae (ra-2), (ra-4), and (ra-6), to reduce the refractive index as well as to inhibit an increase in driving voltage when the organic compound is used as an electron-transport material in a charge-transport device such as a light-emitting device.

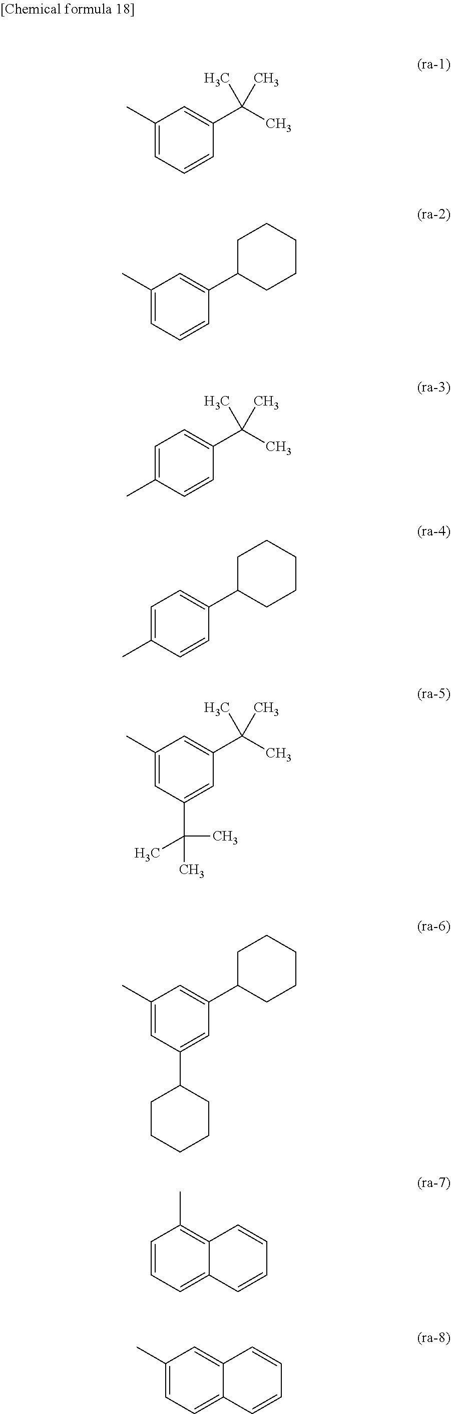

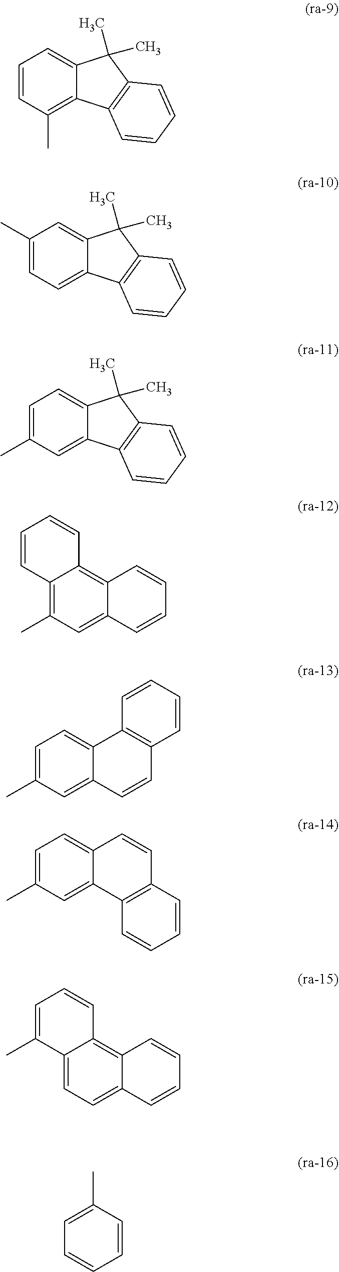

In each of the above structures, the substituted or unsubstituted pyridinyl group in the organic compounds represented by General Formula (G1), General Formula (G2), and General Formula (G3) is preferably an unsubstituted pyridinyl group or a pyridinyl group to which one or more methyl groups are bonded.

In each of the above structures, the alicyclic group is preferably a cycloalkyl group having 3 to 6 carbon atoms in the organic compounds represented by General Formula (G1), General Formula (G2), and General Formula (G3) and the substituted or unsubstituted groups including any one of a pyrimidinyl group, a pyrazinyl group, and a triazinyl group which are represented by Formula (G1-3) and Formula (G1-3-1).

In each of the above structures, the alkyl group having 1 to 6 carbon atoms is preferably a branched alkyl group having 3 to 5 carbon atoms in the organic compounds represented by General Formula (G1), General Formula (G2), and General Formula (G3) and the substituted or unsubstituted groups including any one of a pyrimidinyl group, a pyrazinyl group, and a triazinyl group which are represented by Formula (G1-3) and Formula (G1-3-1).

Another embodiment of the present invention is an organic compound represented by General Formula (G3′).

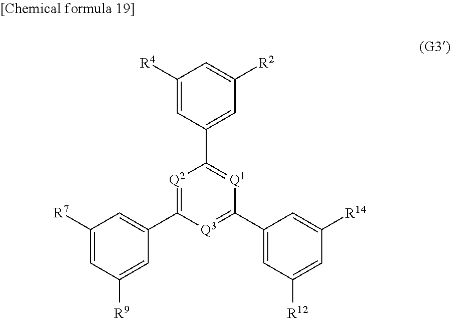

In General Formula (G3′), one to three of Q¹ to Q³ represent N and when one or two of Q¹ to Q³ represent N, the remaining two or one of Q¹ to Q³ represent CH. Furthermore, R² represents a group represented by Formula (R²-1), and R⁴, R⁷, R⁹, R¹², and R¹⁴ each independently represent any one of groups represented by Formulae (r-1) to (r-44). Note that in Formula (R²-1), β represents a substituted or unsubstituted phenylene group or a substituted or unsubstituted biphenyldiyl group, R²⁵ represents any one of the groups represented by Formulae (r-1) to (r-24), and n is 1 or 2. Note that the organic compound represented by General Formula (G3′) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

Note that in the structure of the organic compound represented by General Formula (G3′), the proportion of carbon atoms forming bonds by the sp³ hybrid orbitals affects the refractive index of the organic compound. That is, an increase in the number of carbon atoms forming bonds by the sp³ hybrid orbitals lowers the refractive index; thus, the outcoupling efficiency of a light-emitting device including the organic compound can be improved. Furthermore, the number of carbon atoms forming bonds by the sp³ hybrid orbitals is preferably large, in which case the index of heat resistance such as a glass transition temperature is improved. However, when the number of carbon atoms forming bonds by the sp³ hybrid orbitals is too large, an overlap of LUMO between adjacent molecules of the organic compound is inhibited and thus the carrier-transport property (e.g., electron-transport and electron-injection properties) is lowered. Hence, the proportion of carbon atoms forming bonds by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming bonds by the sp³ hybrid orbitals in the total number of carbon atoms in a molecule of the organic compound is preferably higher than or equal to 20% and lower than or equal to 40%.

The organic compound represented by General Formula (G3′) is preferably formed of only a six-membered heteroaromatic ring/six-membered heteroaromatic rings including Q¹ to Q³, a six-membered aromatic ring/six-membered aromatic rings (i.e., a substituted or unsubstituted phenyl group), and a hydrocarbon group/hydrocarbon groups forming bonds by the sp³ hybrid orbitals (e.g., an alkyl group or an alicyclic group), that is, the organic compound preferably includes no fused ring, in which case the refractive index is lowered and the transport property of carriers (electrons) is increased.

A pyridine ring, a pyrimidine ring, or a triazine ring can be used as the six-membered ring including Q¹ to Q³ in the organic compound represented by General Formula (G3′). In the case where the organic compound represented by General Formula (G3′) is used for a layer in contact with a light-emitting layer or a layer in contact with an active layer, any of a triazine ring, a pyrazine ring, and a pyrimidine ring, which easily inject electrons into these layers and have a high electron-transport property, is preferably used, and a triazine ring is particularly preferable.

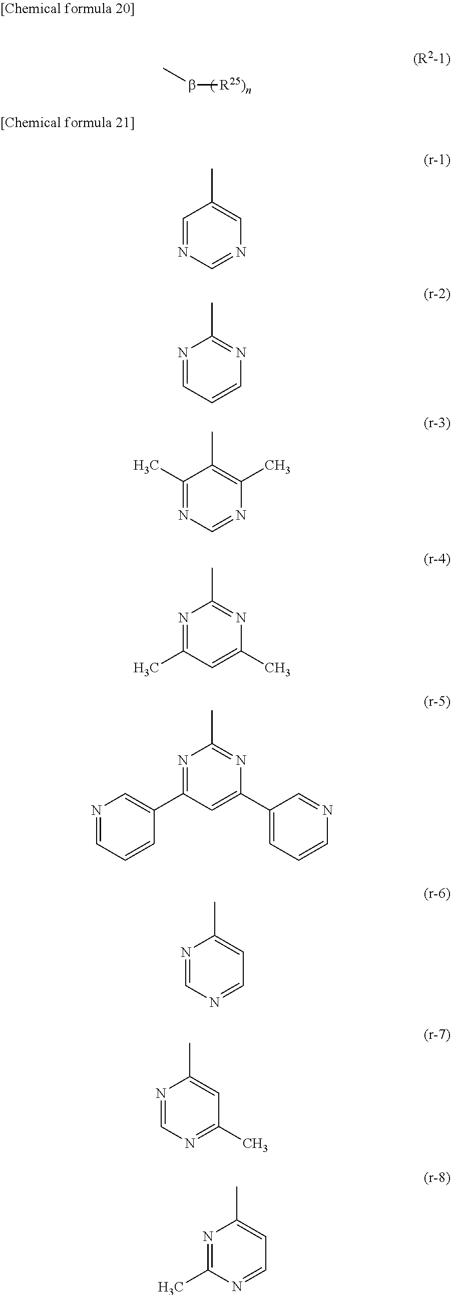

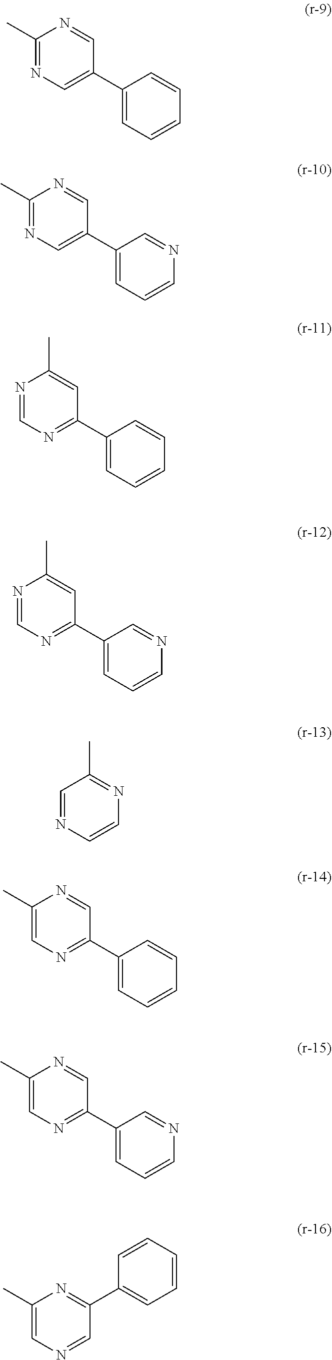

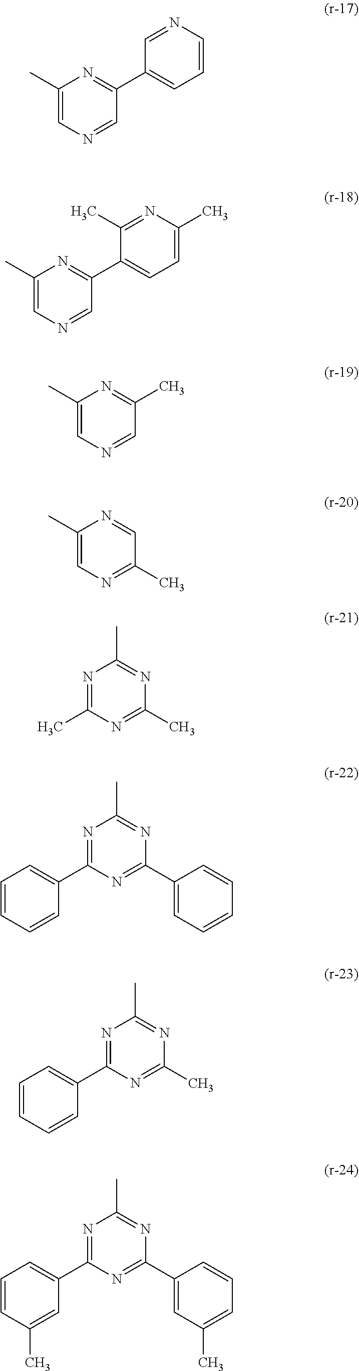

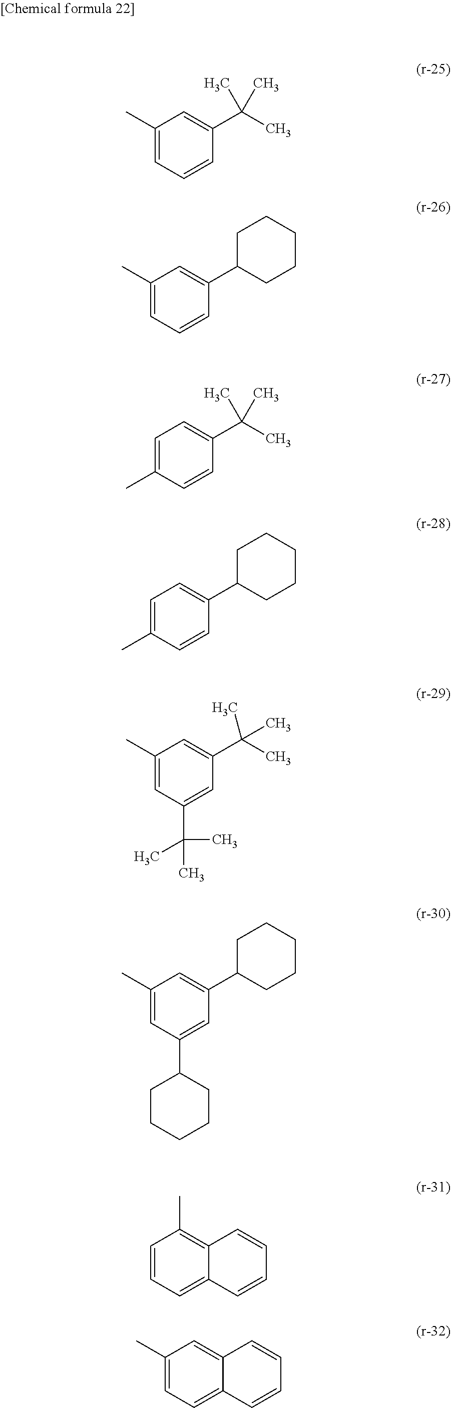

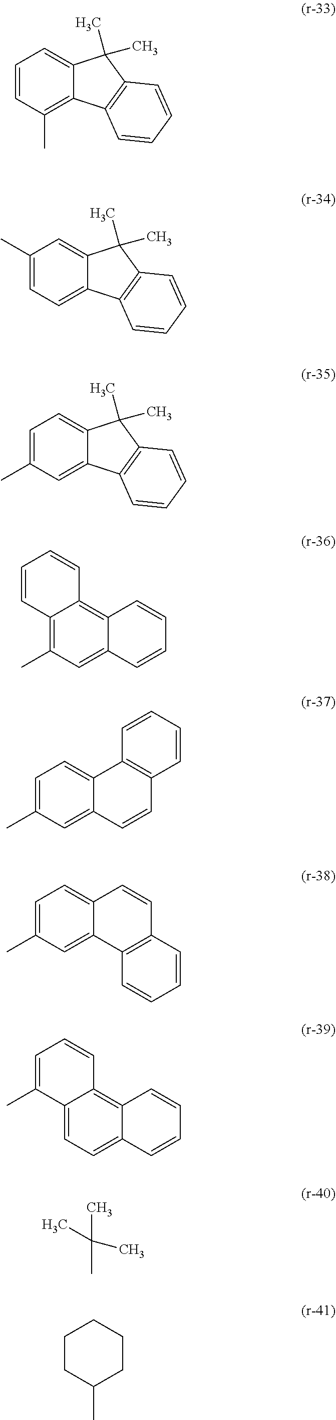

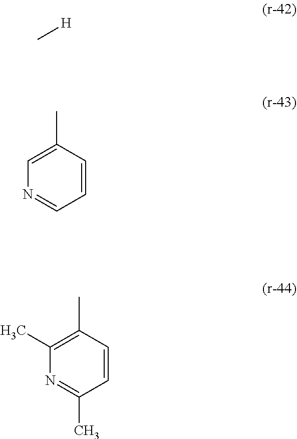

In the above structure, β in the group represented by Formula (R²-1) preferably represents a group represented by any one of Formulae (β-1) to (β-14).

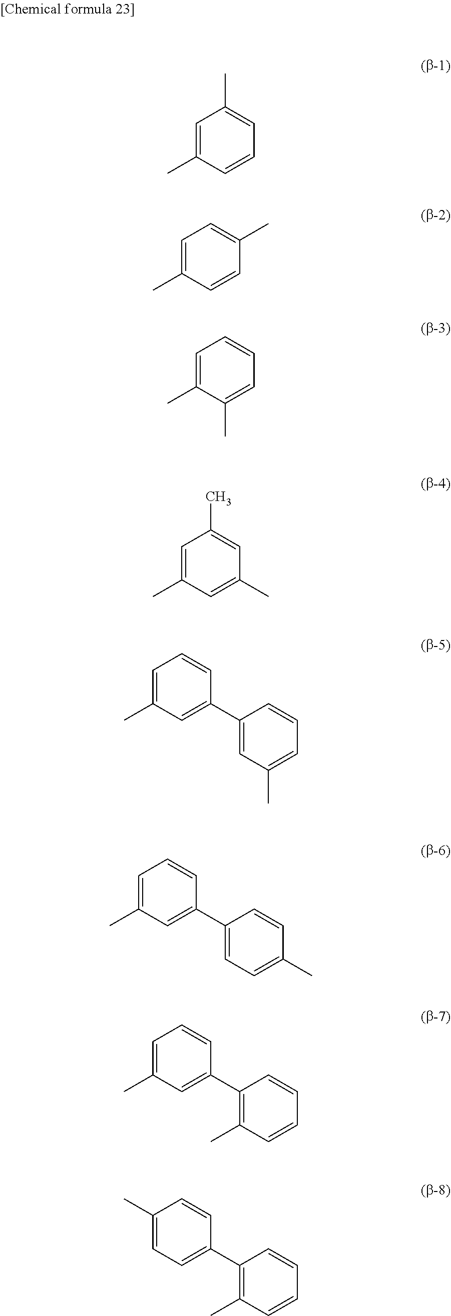

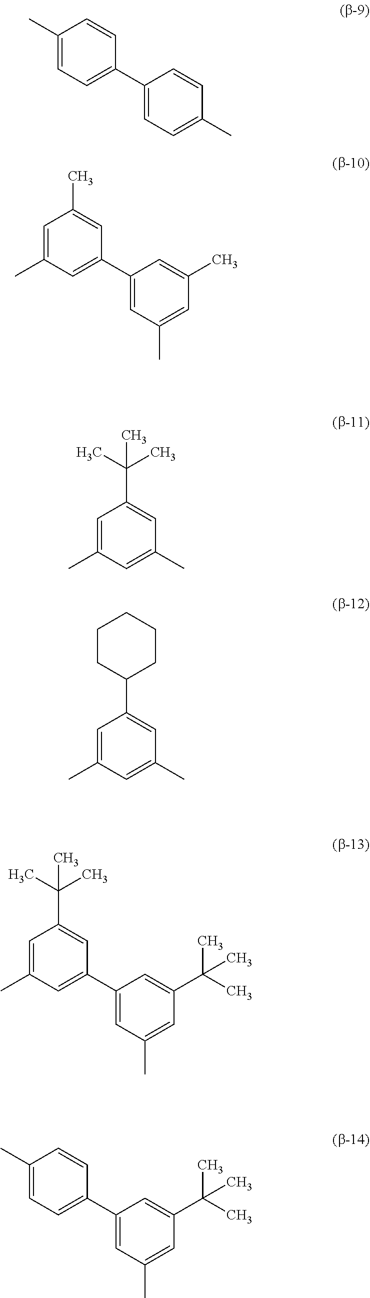

Note that some or all of the hydrogen atoms in the organic compound represented by General Formula (G3′) can be deuterium atoms. In this case, the use of the organic compound in a light-emitting layer, a layer in contact with the light-emitting layer, or the like in a light-emitting device is expected to enable the device to have a long lifetime. The organic compound in which all the hydrogen atoms are protium is also preferable because its synthesis cost can be lower.

Next, specific examples of the organic compounds of embodiments of the present invention having the above structures are shown below.

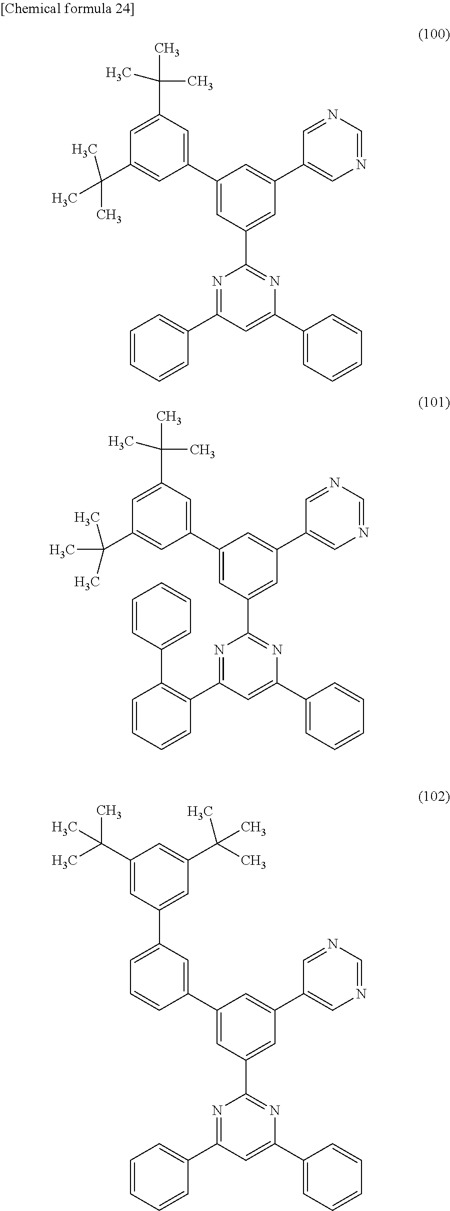

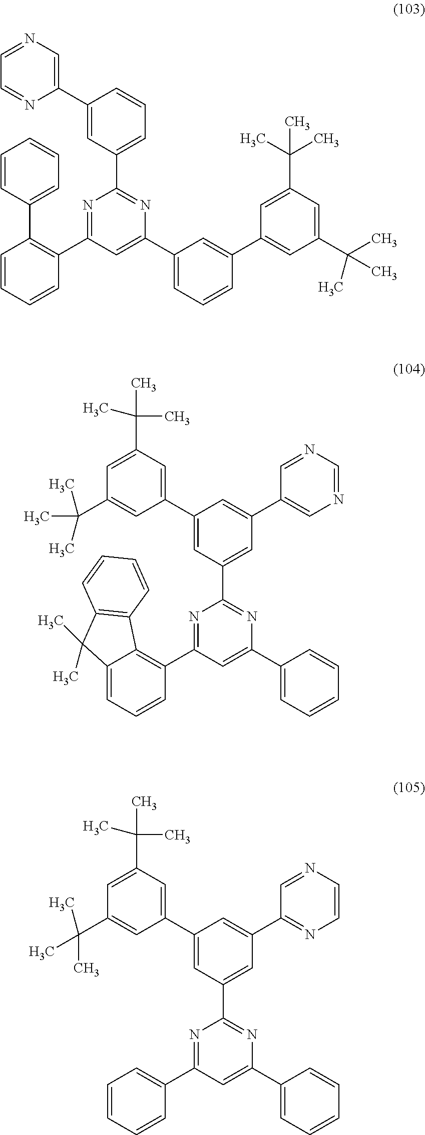

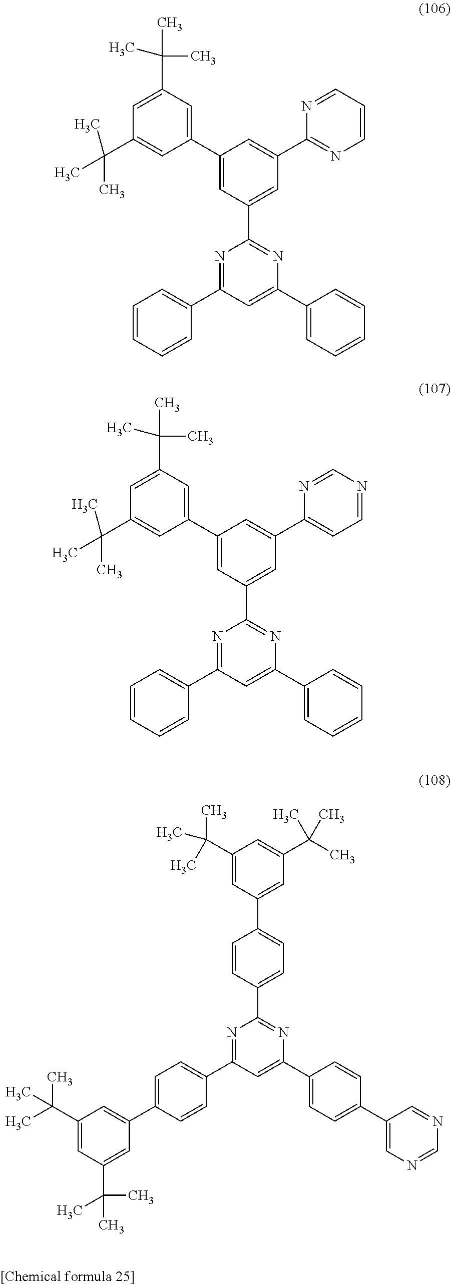 [SMILES: CC(C)(C)c1cc(-c2cc(-c3ncccn3)cc(-c3nc(-c4ccccc4)cc(-c4ccccc4)n3)c2)cc(C(C)(C)C)c1 ; CC(C)(C)c1cc(-c2cc(-c3ccncn3)cc(-c3nc(-c4ccccc4)cc(-c4ccccc4)n3)c2)cc(C(C)(C)C)c1 ; CC(C)(C)c1cc(-c2ccc(-c3cc(-c4ccc(-c5cncnc5)cc4)nc(-c4ccc(-c5cc(C(C)(C)C)cc(C(C)(C)C)c5)cc4)n3)cc2)cc(C(C)(C)C)c1]

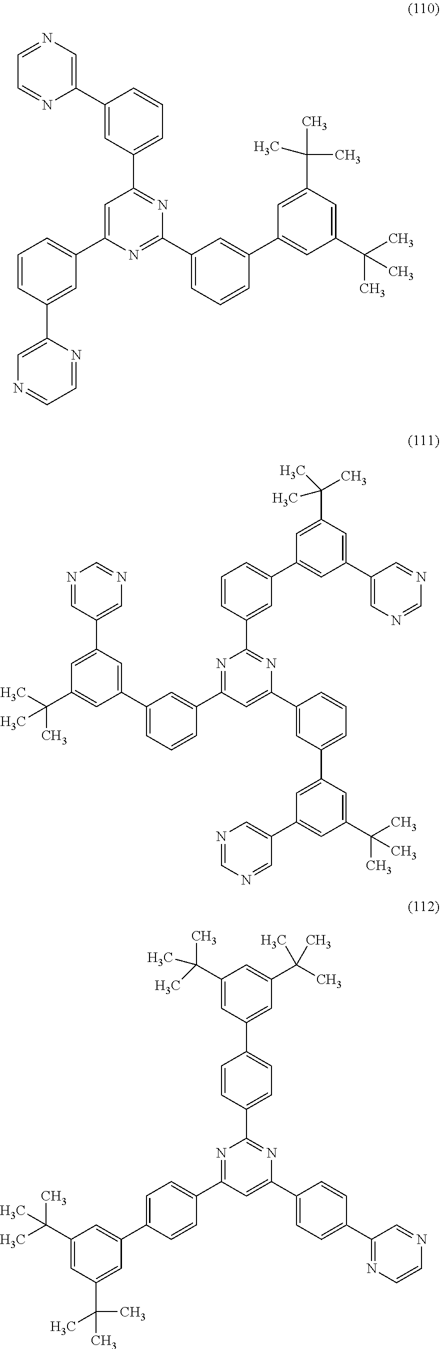

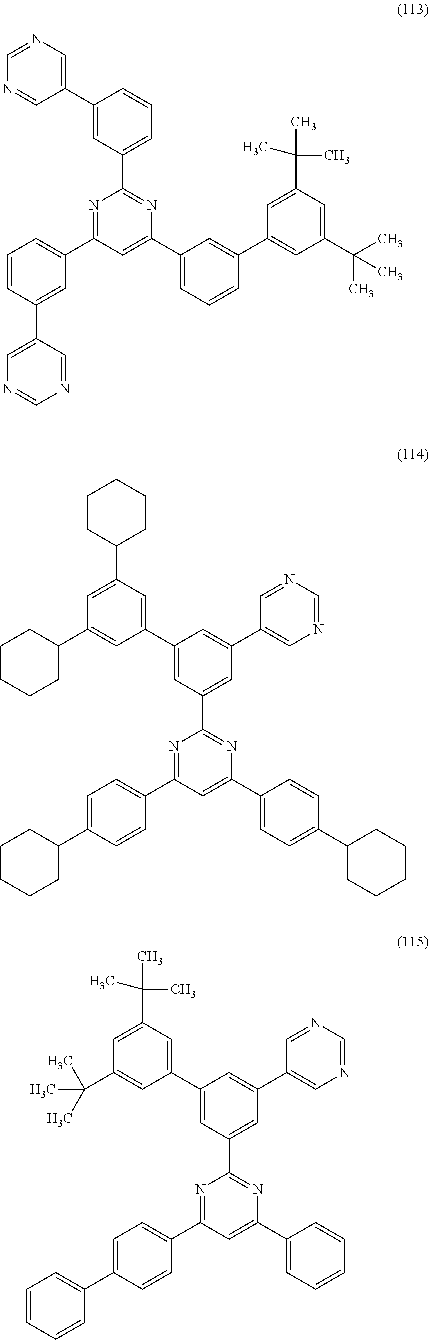 [SMILES: CC(C)(C)c1cc(-c2cccc(-c3cc(-c4cccc(-c5cncnc5)c4)nc(-c4cccc(-c5cncnc5)c4)n3)c2)cc(C(C)(C)C)c1 ; c1ncc(-c2cc(-c3cc(C4CCCCC4)cc(C4CCCCC4)c3)cc(-c3nc(-c4ccc(C5CCCCC5)cc4)cc(-c4ccc(C5CCCCC5)cc4)n3)c2)cn1 ; CC(C)(C)c1cc(-c2cc(-c3cncnc3)cc(-c3nc(-c4ccccc4)cc(-c4ccc(-c5ccccc5)cc4)n3)c2)cc(C(C)(C)C)c1]

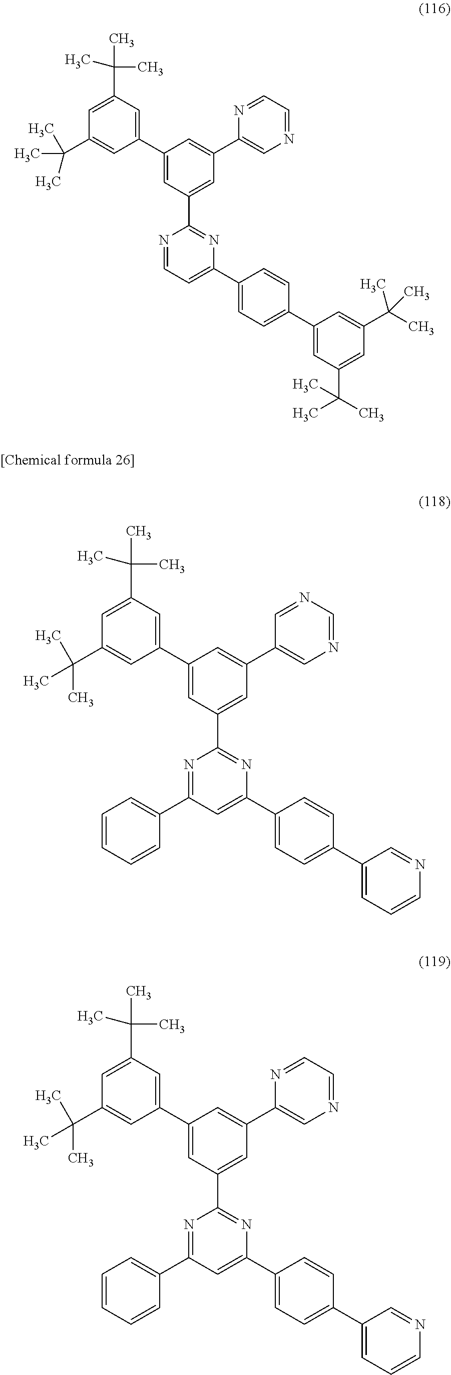

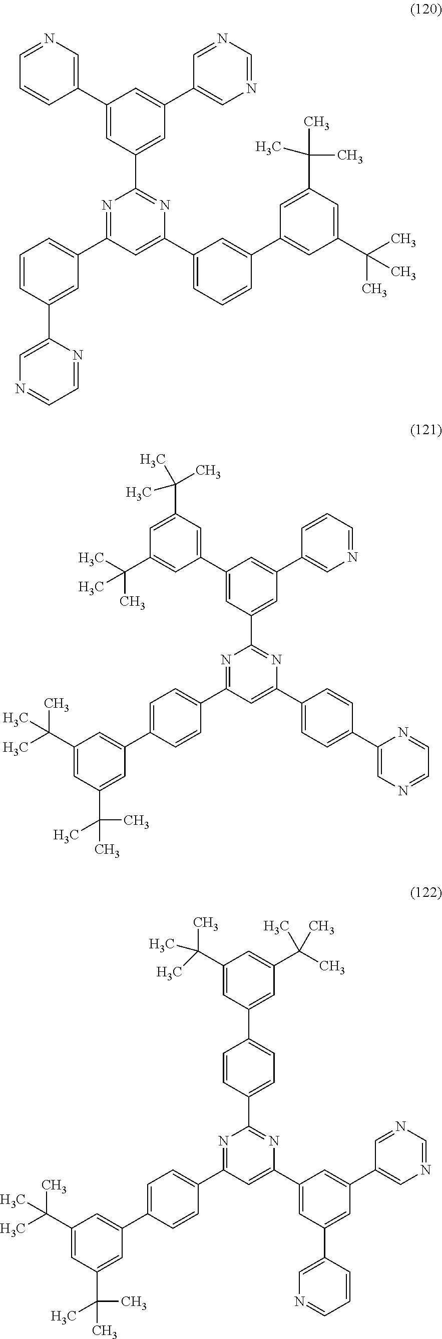

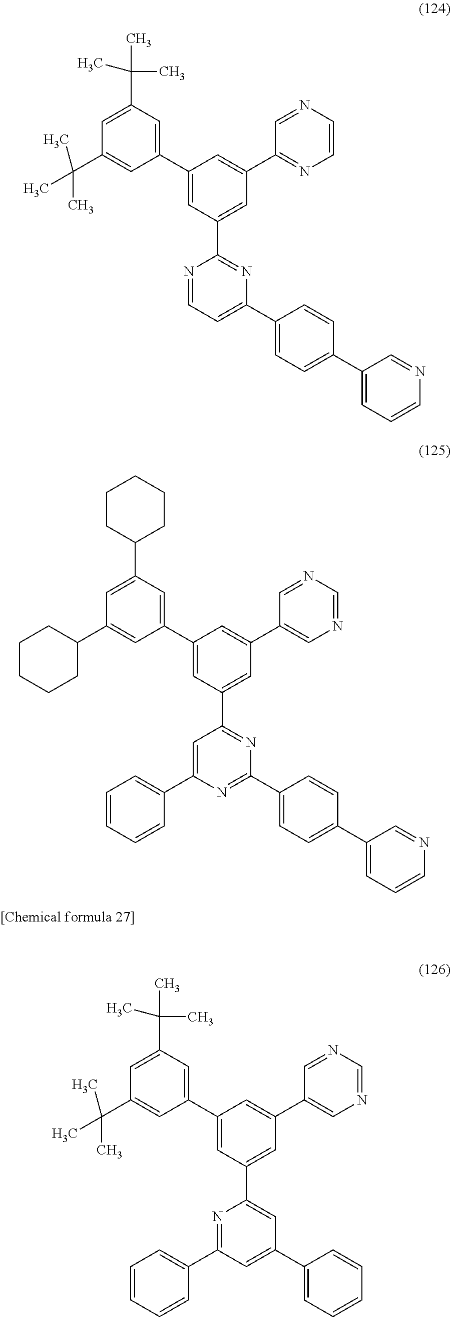 [SMILES: CC(C)(C)c1cc(-c2cc(-c3cnccn3)cc(-c3nccc(-c4ccc(-c5cccnc5)cc4)n3)c2)cc(C(C)(C)C)c1 ; c1ccc(-c2cc(-c3cc(-c4cncnc4)cc(-c4cc(C5CCCCC5)cc(C5CCCCC5)c4)c3)nc(-c3ccc(-c4cccnc4)cc3)n2)cc1 ; CC(C)(C)c1cc(-c2cc(-c3cncnc3)cc(-c3cc(-c4ccccc4)cc(-c4ccccc4)n3)c2)cc(C(C)(C)C)c1]

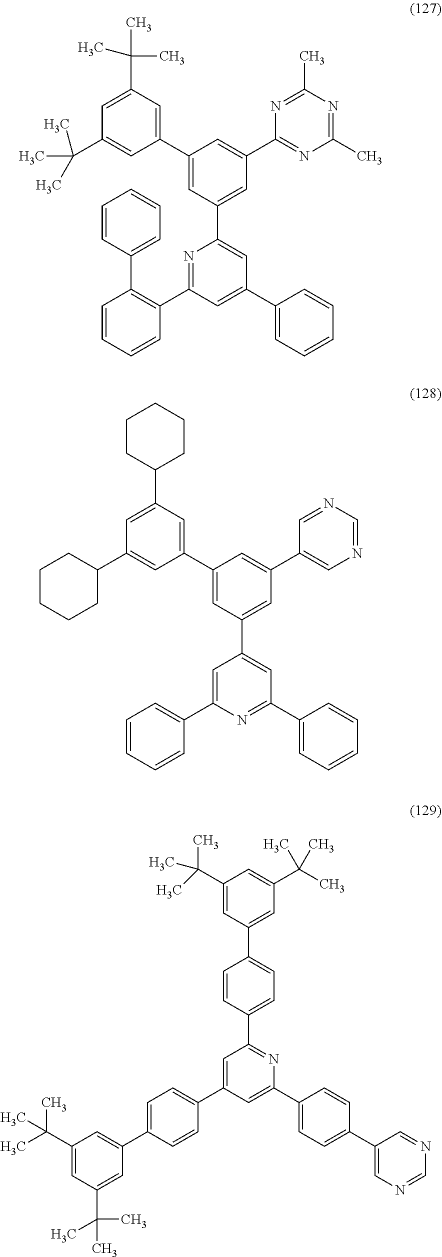

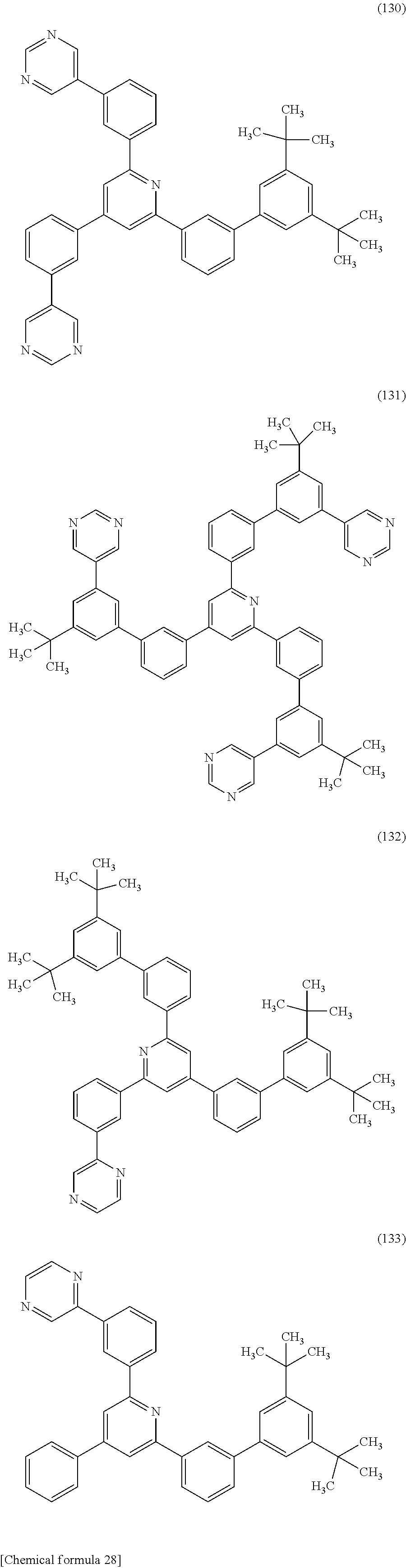

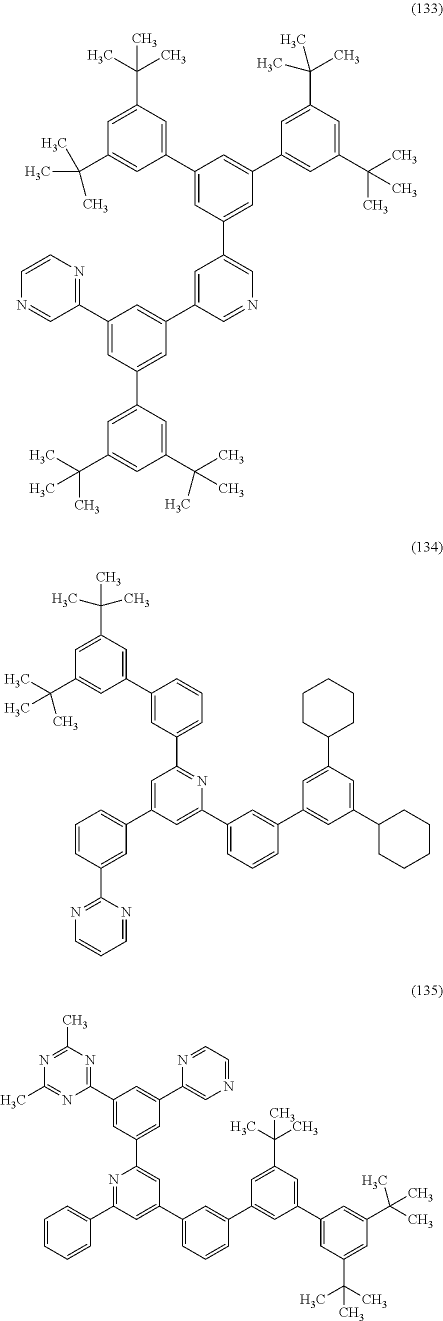

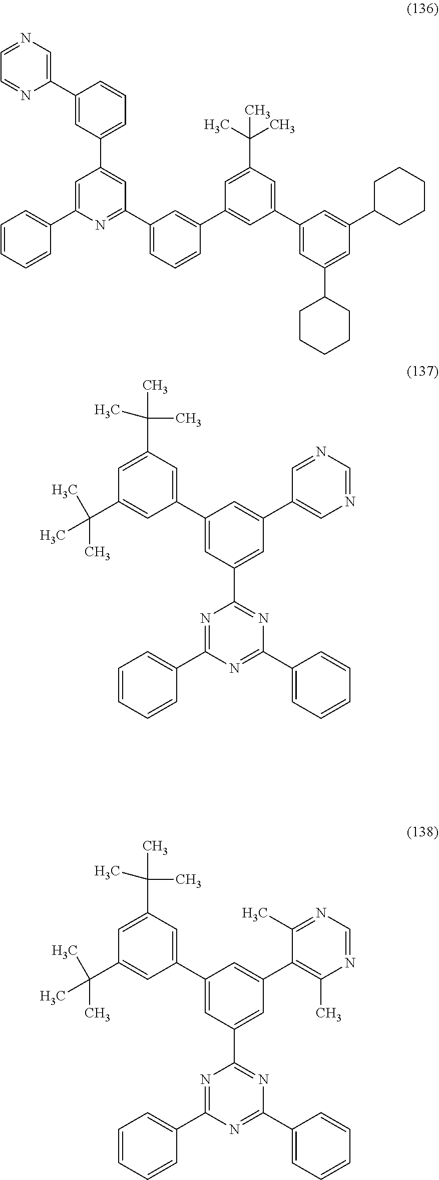 [SMILES: CC(C)(C)c1cc(-c2cccc(-c3cc(-c4cccc(-c5cnccn5)c4)cc(-c4ccccc4)n3)c2)cc(-c2cc(C3CCCCC3)cc(C3CCCCC3)c2)c1 ; CC(C)(C)c1cc(-c2cc(-c3cncnc3)cc(-c3nc(-c4ccccc4)nc(-c4ccccc4)n3)c2)cc(C(C)(C)C)c1 ; Cc1ncnc(C)c1-c1cc(-c2cc(C(C)(C)C)cc(C(C)(C)C)c2)cc(-c2nc(-c3ccccc3)nc(-c3ccccc3)n2)c1]

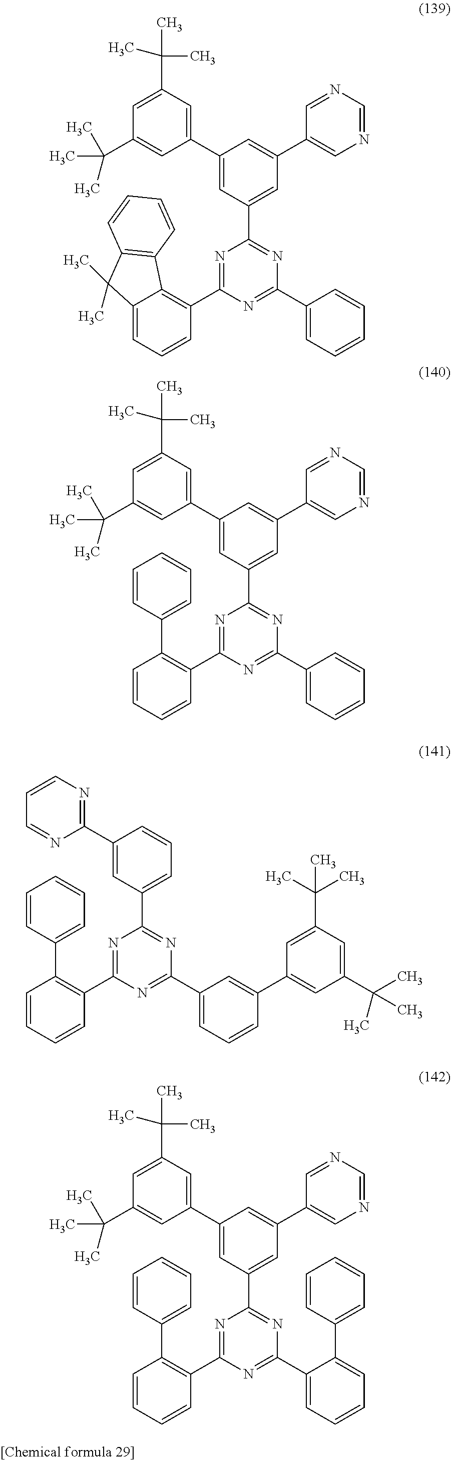

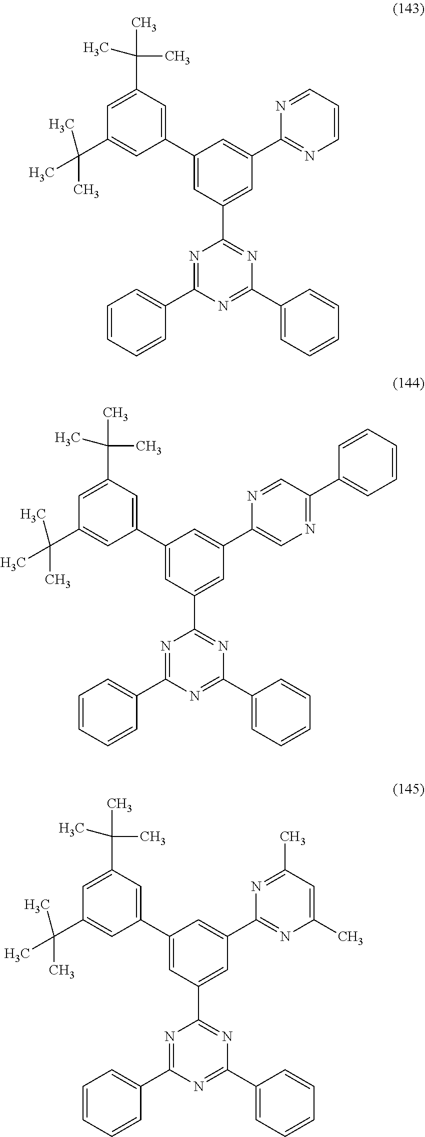 [SMILES: CC(C)(C)c1cc(-c2cc(-c3ncccn3)cc(-c3nc(-c4ccccc4)nc(-c4ccccc4)n3)c2)cc(C(C)(C)C)c1 ; CC(C)(C)c1cc(-c2cc(-c3cnc(-c4ccccc4)cn3)cc(-c3nc(-c4ccccc4)nc(-c4ccccc4)n3)c2)cc(C(C)(C)C)c1 ; Cc1cc(C)nc(-c2cc(-c3cc(C(C)(C)C)cc(C(C)(C)C)c3)cc(-c3nc(-c4ccccc4)nc(-c4ccccc4)n3)c2)n1]

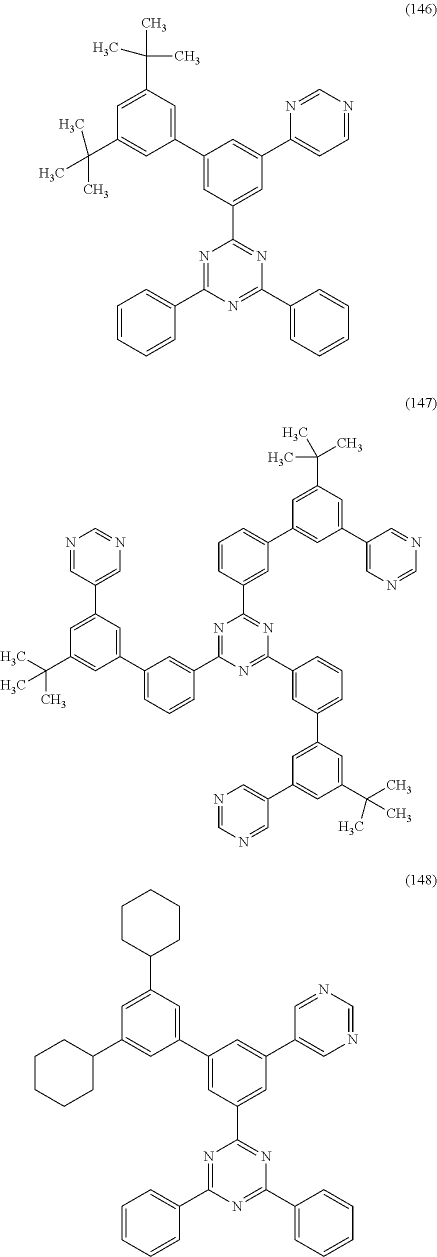 [SMILES: CC(C)(C)c1cc(-c2cc(-c3ccncn3)cc(-c3nc(-c4ccccc4)nc(-c4ccccc4)n3)c2)cc(C(C)(C)C)c1 ; CC(C)(C)c1cc(-c2cncnc2)cc(-c2cccc(-c3nc(-c4cccc(-c5cc(-c6cncnc6)cc(C(C)(C)C)c5)c4)nc(-c4cccc(-c5cc(-c6cncnc6)cc(C(C)(C)C)c5)c4)n3)c2)c1 ; c1ccc(-c2nc(-c3ccccc3)nc(-c3cc(-c4cncnc4)cc(-c4cc(C5CCCCC5)cc(C5CCCCC5)c4)c3)n2)cc1]

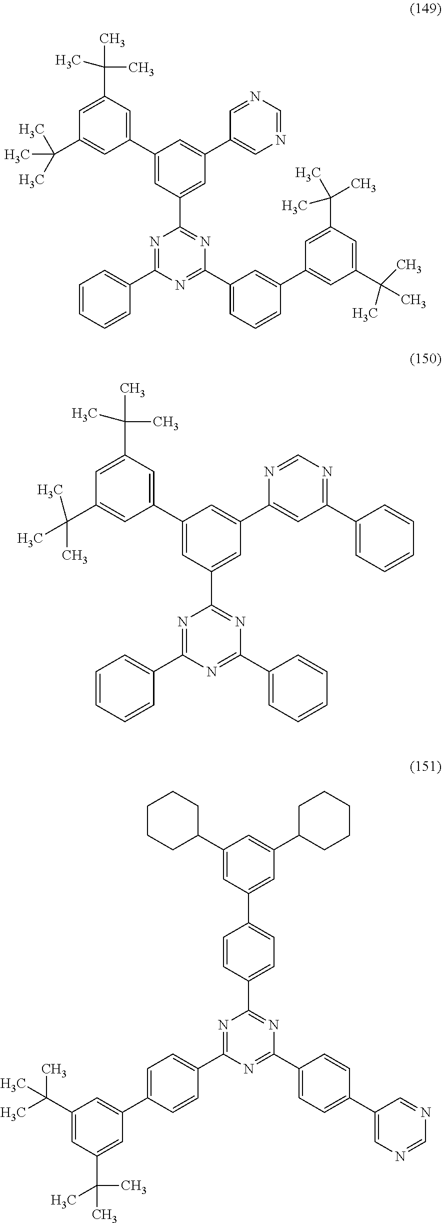

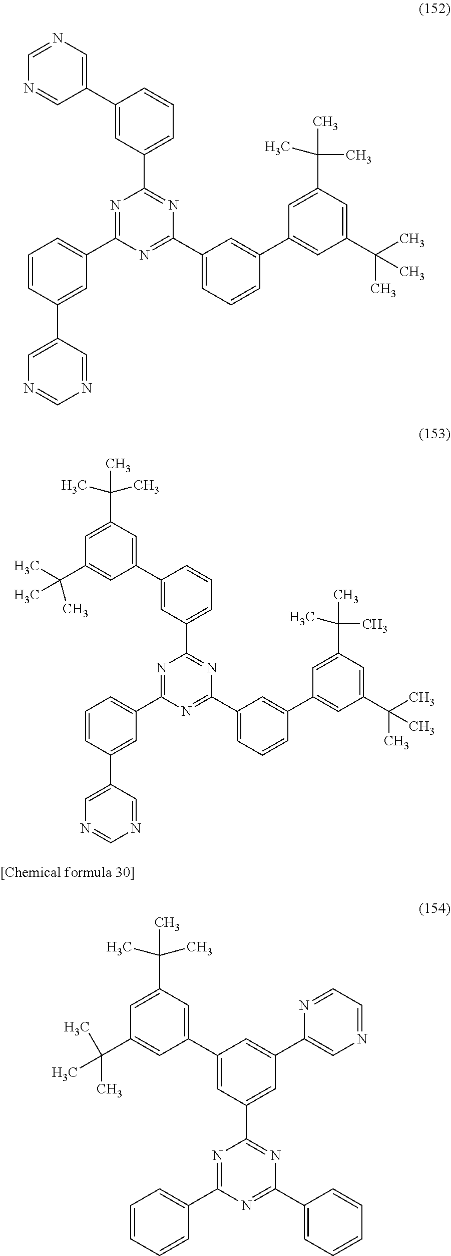

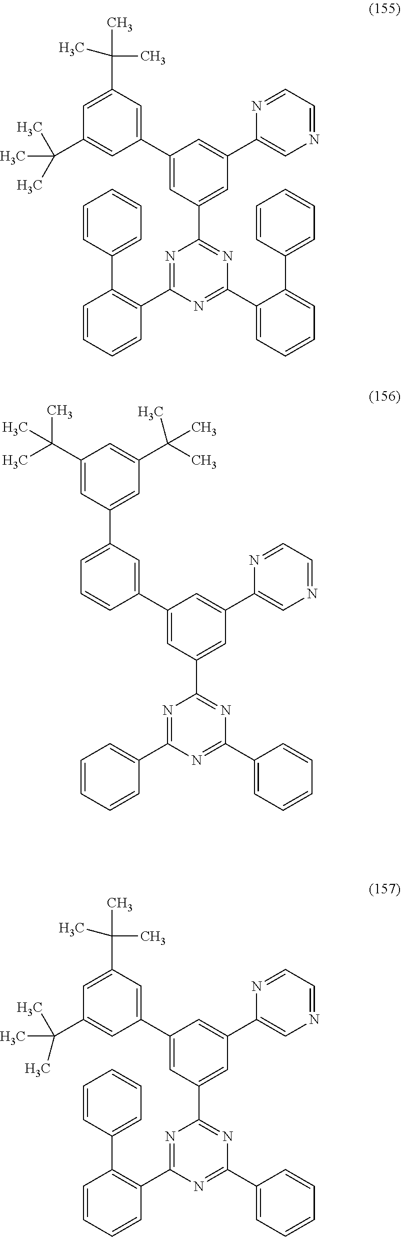 [SMILES: CC(C)(C)c1cc(-c2cc(-c3cnccn3)cc(-c3nc(-c4ccccc4-c4ccccc4)nc(-c4ccccc4-c4ccccc4)n3)c2)cc(C(C)(C)C)c1 ; CC(C)(C)c1cc(-c2cccc(-c3cc(-c4cnccn4)cc(-c4nc(-c5ccccc5)nc(-c5ccccc5)n4)c3)c2)cc(C(C)(C)C)c1 ; CC(C)(C)c1cc(-c2cc(-c3cnccn3)cc(-c3nc(-c4ccccc4)nc(-c4ccccc4-c4ccccc4)n3)c2)cc(C(C)(C)C)c1]

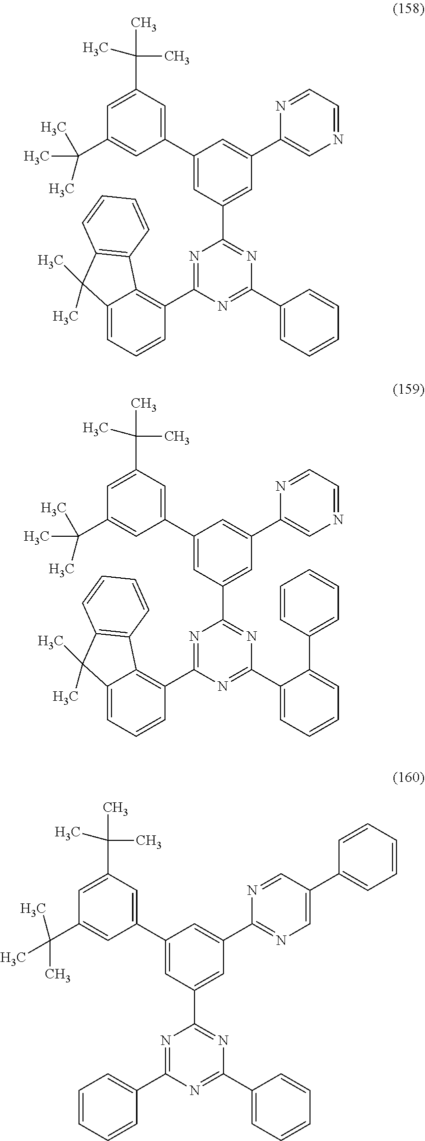 [SMILES: CC(C)(C)c1cc(-c2cc(-c3cnccn3)cc(-c3nc(-c4ccccc4)nc(-c4cccc5c4-c4ccccc4C5(C)C)n3)c2)cc(C(C)(C)C)c1 ; CC(C)(C)c1cc(-c2cc(-c3cnccn3)cc(-c3nc(-c4ccccc4-c4ccccc4)nc(-c4cccc5c4-c4ccccc4C5(C)C)n3)c2)cc(C(C)(C)C)c1 ; CC(C)(C)c1cc(-c2cc(-c3ncc(-c4ccccc4)cn3)cc(-c3nc(-c4ccccc4)nc(-c4ccccc4)n3)c2)cc(C(C)(C)C)c1]

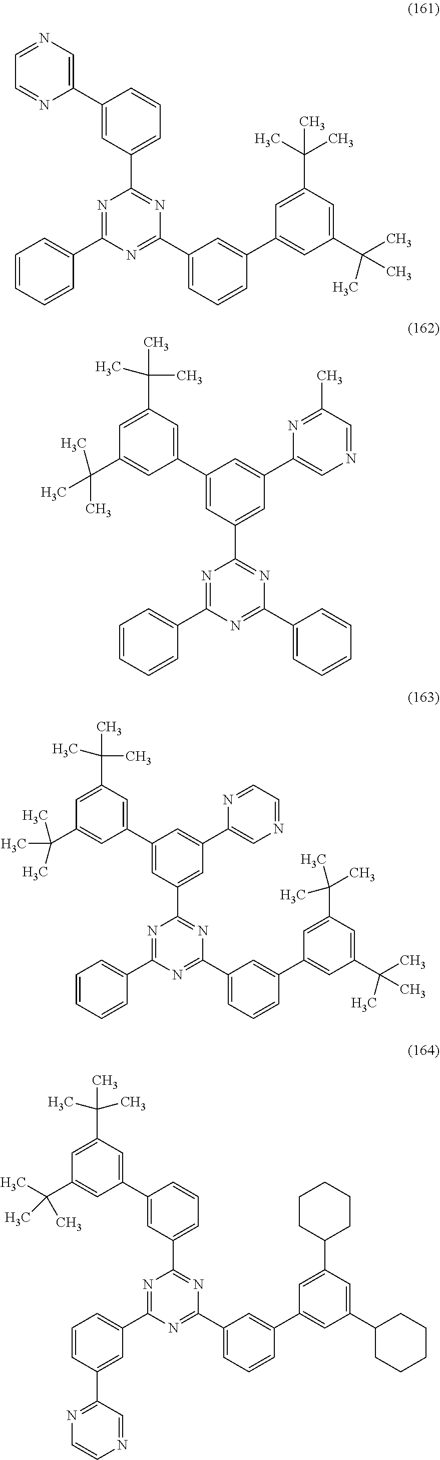

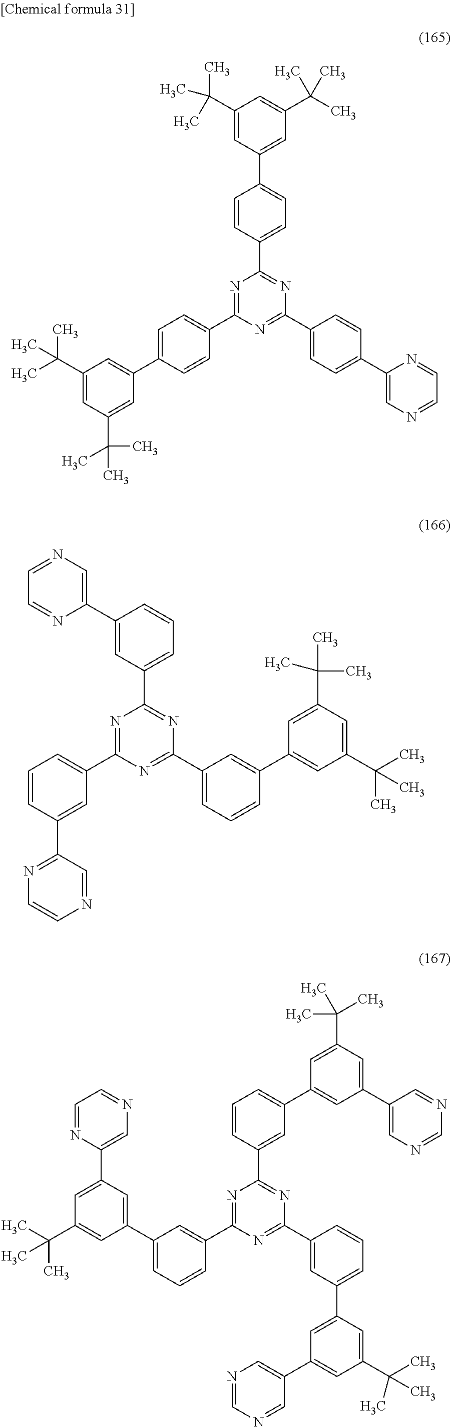

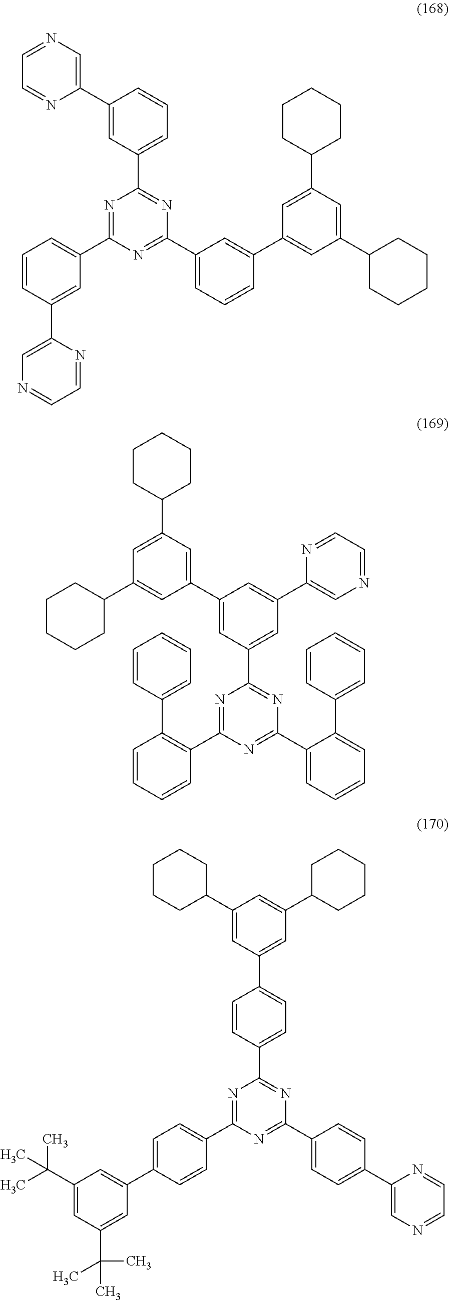 [SMILES: c1cc(-c2cc(C3CCCCC3)cc(C3CCCCC3)c2)cc(-c2nc(-c3cccc(-c4cnccn4)c3)nc(-c3cccc(-c4cnccn4)c3)n2)c1 ; c1ccc(-c2ccccc2-c2nc(-c3cc(-c4cc(C5CCCCC5)cc(C5CCCCC5)c4)cc(-c4cnccn4)c3)nc(-c3ccccc3-c3ccccc3)n2)cc1 ; CC(C)(C)c1cc(-c2ccc(-c3nc(-c4ccc(-c5cc(C6CCCCC6)cc(C6CCCCC6)c5)cc4)nc(-c4ccc(-c5cnccn5)cc4)n3)cc2)cc(C(C)(C)C)c1]

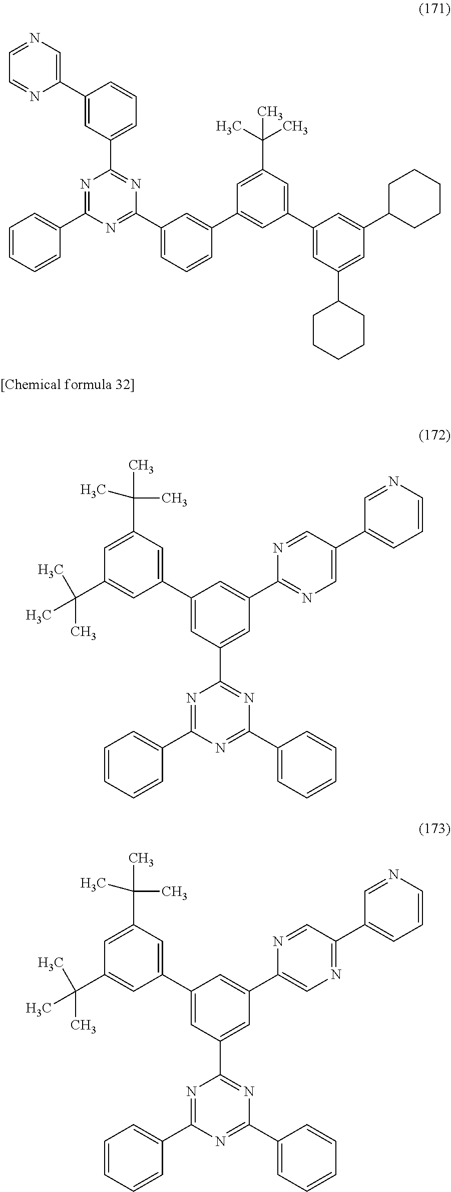 [SMILES: CC(C)(C)c1cc(-c2cccc(-c3nc(-c4ccccc4)nc(-c4cccc(-c5cnccn5)c4)n3)c2)cc(-c2cc(C3CCCCC3)cc(C3CCCCC3)c2)c1 ; CC(C)(C)c1cc(-c2cc(-c3ncc(-c4cccnc4)cn3)cc(-c3nc(-c4ccccc4)nc(-c4ccccc4)n3)c2)cc(C(C)(C)C)c1 ; CC(C)(C)c1cc(-c2cc(-c3cnc(-c4cccnc4)cn3)cc(-c3nc(-c4ccccc4)nc(-c4ccccc4)n3)c2)cc(C(C)(C)C)c1]

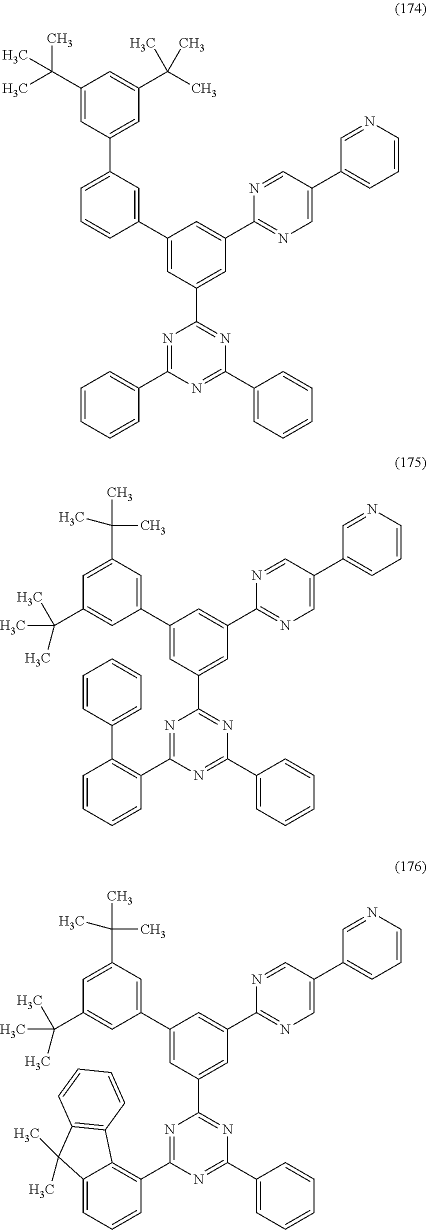 [SMILES: CC(C)(C)c1cc(-c2cccc(-c3cc(-c4ncc(-c5cccnc5)cn4)cc(-c4nc(-c5ccccc5)nc(-c5ccccc5)n4)c3)c2)cc(C(C)(C)C)c1 ; CC(C)(C)c1cc(-c2cc(-c3ncc(-c4cccnc4)cn3)cc(-c3nc(-c4ccccc4)nc(-c4ccccc4-c4ccccc4)n3)c2)cc(C(C)(C)C)c1 ; CC(C)(C)c1cc(-c2cc(-c3ncc(-c4cccnc4)cn3)cc(-c3nc(-c4ccccc4)nc(-c4cccc5c4-c4ccccc4C5(C)C)n3)c2)cc(C(C)(C)C)c1]

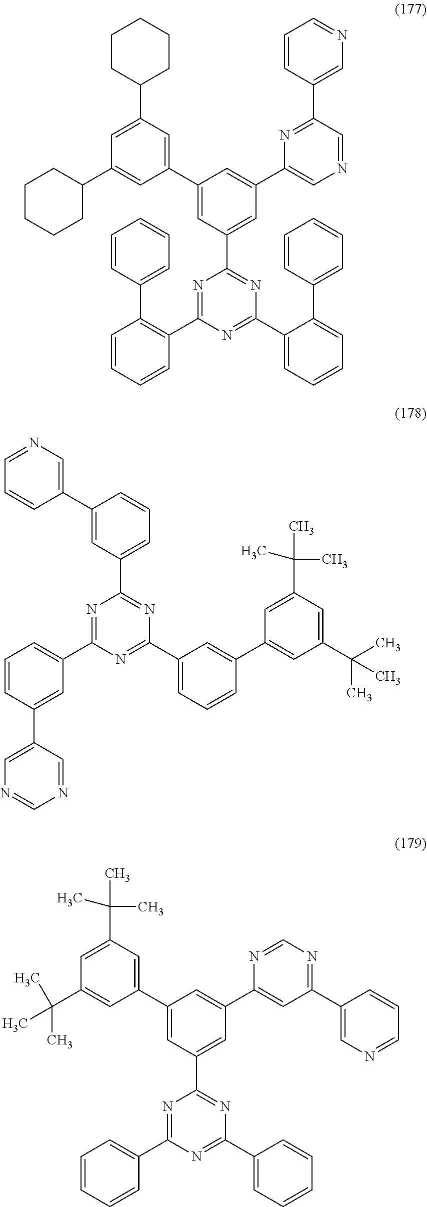

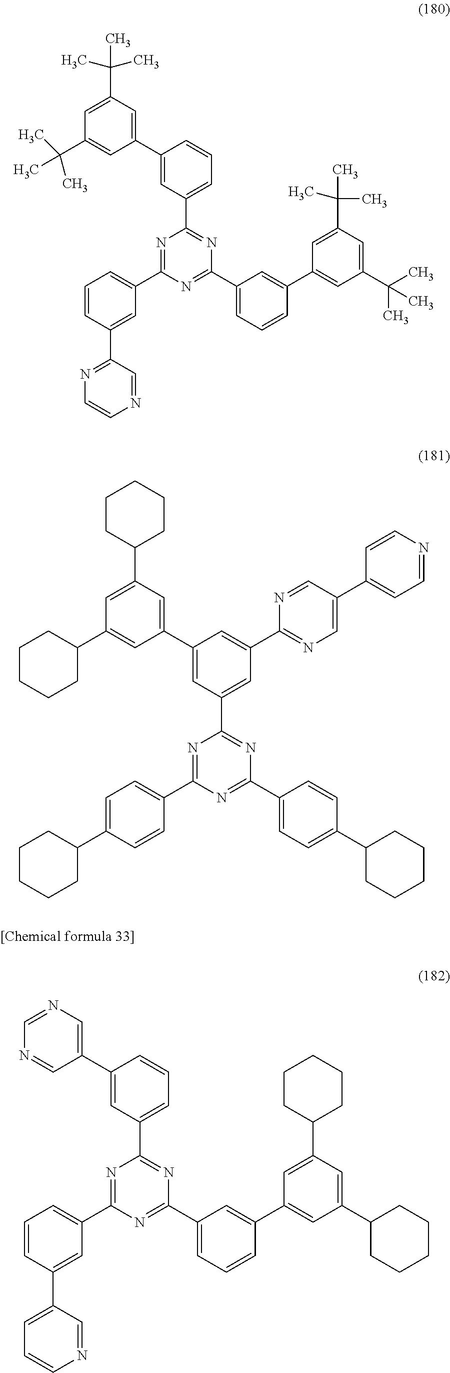 [SMILES: CC(C)(C)c1cc(-c2cccc(-c3nc(-c4cccc(-c5cc(C(C)(C)C)cc(C(C)(C)C)c5)c4)nc(-c4cccc(-c5cnccn5)c4)n3)c2)cc(C(C)(C)C)c1 ; c1cc(-c2cnc(-c3cc(-c4cc(C5CCCCC5)cc(C5CCCCC5)c4)cc(-c4nc(-c5ccc(C6CCCCC6)cc5)nc(-c5ccc(C6CCCCC6)cc5)n4)c3)nc2)ccn1 ; c1cncc(-c2cccc(-c3nc(-c4cccc(-c5cncnc5)c4)nc(-c4cccc(-c5cc(C6CCCCC6)cc(C6CCCCC6)c5)c4)n3)c2)c1]

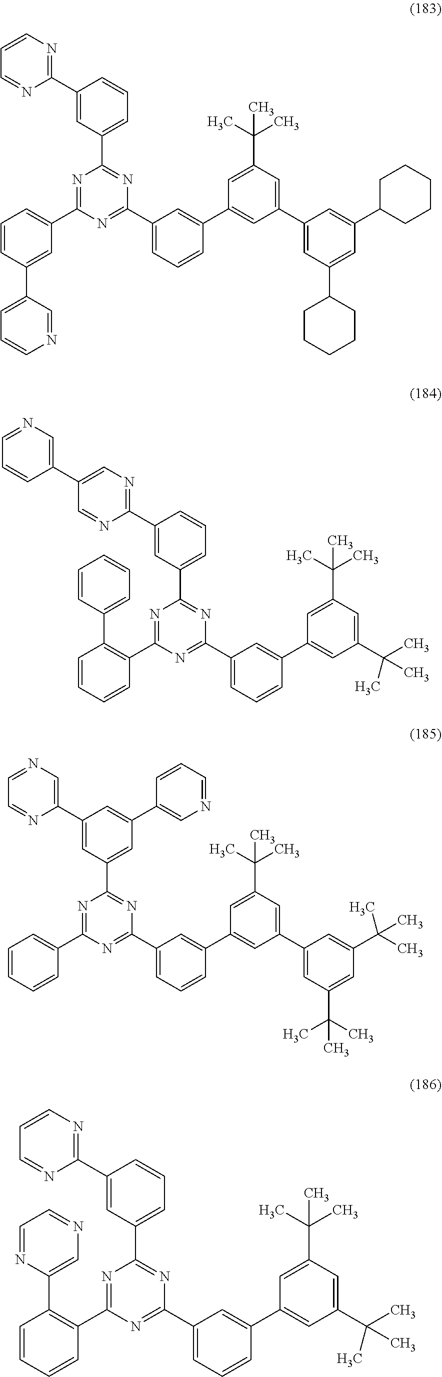 [SMILES: CC(C)(C)c1cc(-c2cccc(-c3nc(-c4cccc(-c5cccnc5)c4)nc(-c4cccc(-c5ncccn5)c4)n3)c2)cc(-c2cc(C3CCCCC3)cc(C3CCCCC3)c2)c1 ; CC(C)(C)c1cc(-c2cccc(-c3nc(-c4cccc(-c5ncc(-c6cccnc6)cn5)c4)nc(-c4ccccc4-c4ccccc4)n3)c2)cc(C(C)(C)C)c1 ; CC(C)(C)c1cc(-c2cccc(-c3nc(-c4ccccc4)nc(-c4cc(-c5cccnc5)cc(-c5cnccn5)c4)n3)c2)cc(-c2cc(C(C)(C)C)cc(C(C)(C)C)c2)c1 ; CC(C)(C)c1cc(-c2cccc(-c3nc(-c4cccc(-c5ncccn5)c4)nc(-c4ccccc4-c4cnccn4)n3)c2)cc(C(C)(C)C)c1]

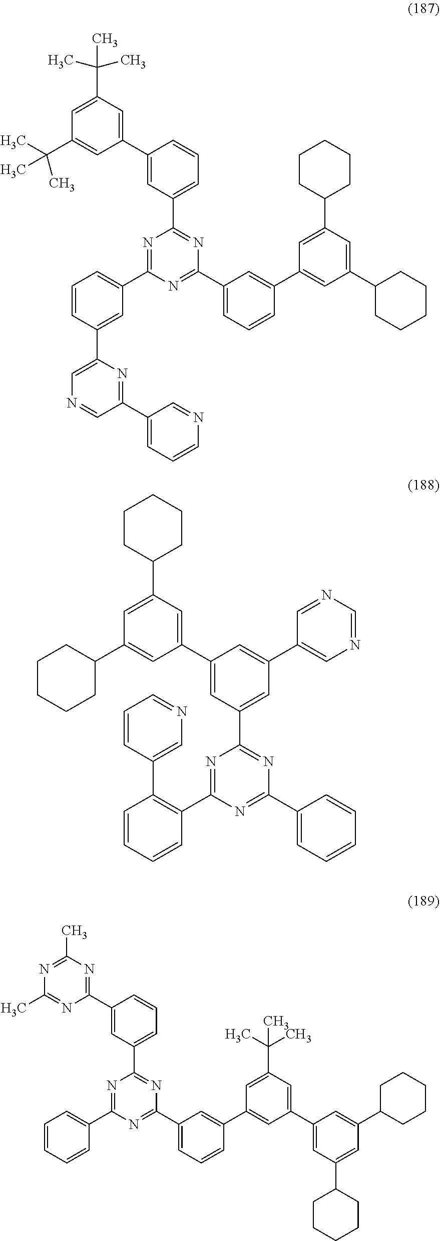

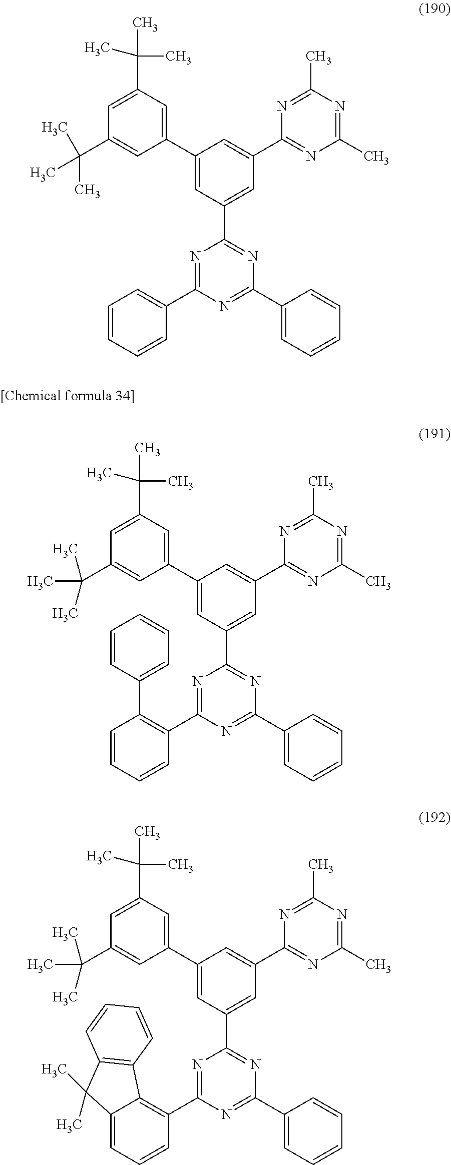

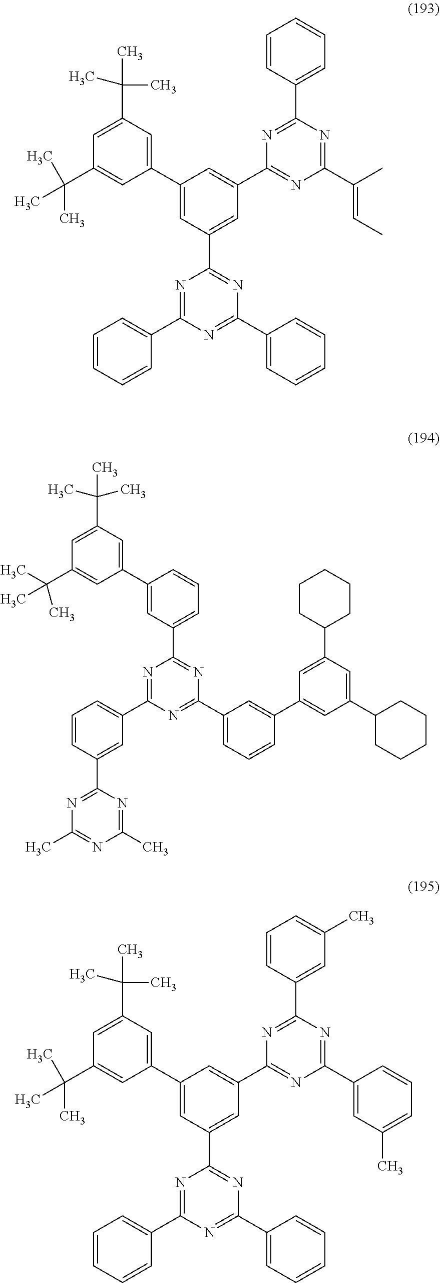

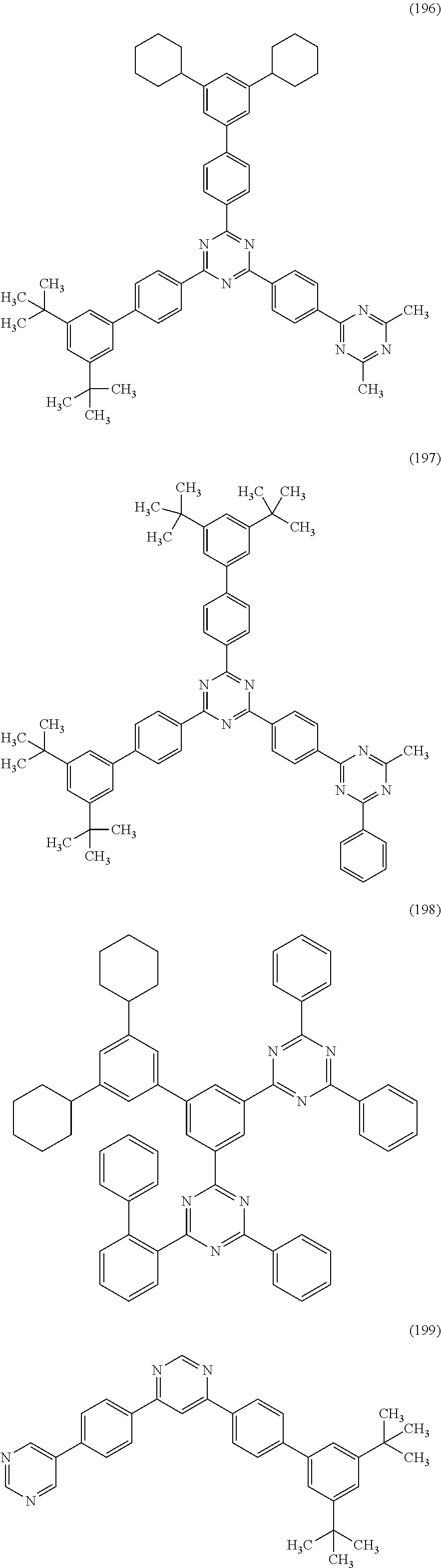

For example, Structural Formula (138) represents the organic compound of General Formula (G3) in which R² represents a pyrimidinyl group including two methyl groups as substituents and R⁴ represents a phenyl group including two tert-butyl groups as substituents.

For example, Structural Formula (111), Structural Formula (131), and Structural Formula (147) each represent the organic compound of General Formula (G3) in which R², R⁷, and R¹² each represent a pyrimidinyl-tert-butylphenylene group that is the group including a pyrimidinyl group. In other words, Structural Formula (111), Structural Formula (131), and Structural Formula (147) each represent the organic compound of General Formula (G3) in which the group including a pyrimidinyl group is represented by Formula (G1-3) in which R²¹ represents hydrogen, n is 1, R²³ represents hydrogen, and R²⁴ represents a pyrimidinyl group.

The organic compounds represented by Structural Formulae (100) to (116), (118) to (122), and (124) to (199) are examples of the organic compound represented by General Formula (G1). The organic compound of one embodiment of the present invention is not limited thereto.

Next, a method for synthesizing the organic compound of one embodiment of the present invention represented by General Formula (G1) will be described.

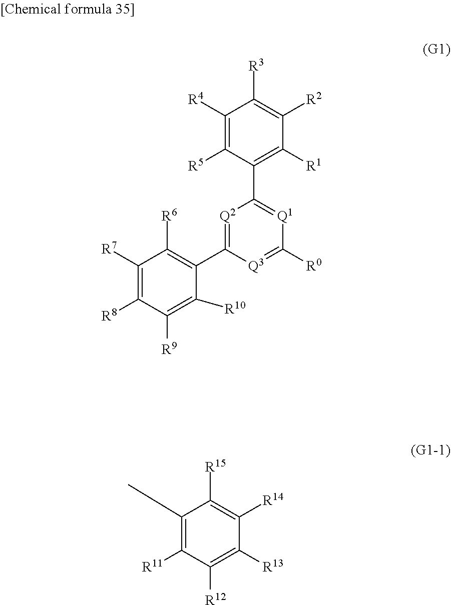

In General Formula (G1), one to three of Q¹ to Q³ represent N and when one or two of Q¹ to Q³ represent N, the remaining two or one of Q¹ to Q³ represent CH. Furthermore, R⁰ represents any of hydrogen, an alkyl group having 1 to 6 carbon atoms, an alicyclic group having 3 to 10 carbon atoms, and a group represented by Formula (G1-1). At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Note that the organic compound represented by General Formula (G1) includes a plurality of hydrocarbon groups each independently selected from an alkyl group having 1 to 6 carbon atoms and an alicyclic group having 3 to 10 carbon atoms, and the proportion of carbon atoms forming bonds by the sp³ hybrid orbitals in the total number of carbon atoms in a molecule of the organic compound is higher than or equal to 10% and lower than or equal to 60%.

### <<Method for Synthesizing Organic Compound Represented by General Formula (G1)>>

An example of a synthesis method of the organic compound represented by General Formula (G1) is described below. A variety of reactions can be used for the synthesis of this organic compound. For example, as shown in Synthesis Scheme (A-1), an arylboron compound (a1) and a heteroaryl halide (a2) are coupled, whereby the target compound (G1) can be synthesized. For this reaction, a synthesis method in which a metal catalyst is used under the presence of a base, e.g., the Suzuki-Miyaura reaction, can be used.

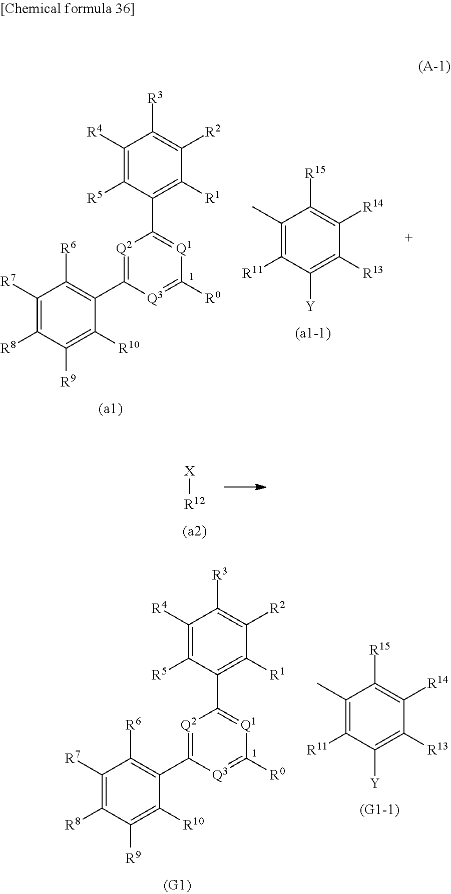

In Synthesis Scheme (A-1), one to three of Q¹ to Q³ represent N and when one or two of Q¹ to Q³ represent N, the remaining two or one of Q¹ to Q³ represent CH. Furthermore, R⁰ in Formula (a1) represents any of hydrogen, an alkyl group having 1 to 6 carbon atoms, an alicyclic group having 3 to 10 carbon atoms, and a group represented by Formula (a1-1), and R⁰ in Formula (G1) represents any of hydrogen, an alkyl group having 1 to 6 carbon atoms, an alicyclic group having 3 to 10 carbon atoms, and a group represented by Formula (G1-1). At least one of R¹ to R¹⁵ represents a substituted or unsubstituted group including any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group. When having a substituent, the substituted or unsubstituted group including any one of a pyrimidinyl group, a pyrazinyl group, and a triazinyl group has one or more substituents, and the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group. Y represents a boronic acid or a boronic ester such as pinacol boron. X represents any of chlorine, bromine, iodine, and a sulfonyloxy group, and an element with a larger atomic number is preferably used to increase reactivity. Alternatively, X may be a boronic acid or a boronic ester such as pinacol boron and Y may be a halogen or a sulfonyloxy group in the reaction.

This synthesis scheme shows a reaction example in which R¹² of the heteroaryl halide (a2) is substituted for Y of the group (a1-1) of the arylboron compound (a1). The site of substitution in the reaction may be any of R¹ to R¹¹ and R¹³ to R¹⁵ of the arylboron compound (a1). That is, the arylboron compound (a1) in which one or more of R¹ to R¹¹ and R¹³ to R¹⁵ is Y (a boronic acid or a boronic ester such as pinacol boron) may be reacted with a compound including one or more of R¹ to R¹¹ and R¹³ to R¹⁵ and X (a halogen or a sulfonyloxy group) to synthesize the target compound (G1).

Note that in the case where R⁰ in General Formula (G1) represents hydrogen, an alkyl group having 1 to 6 carbon atoms, or an alicyclic group having 3 to 10 carbon atoms, the arylboron compound (a1) in which any one of R¹ to R¹⁰ is Y may be reacted with the heteroaryl halide (a2) as in Synthesis Scheme (A-1).

In the case of conducting the Suzuki-Miyaura reaction using a palladium catalyst in Synthesis Scheme (A-1), a palladium compound such as tetrakis(triphenylphosphine)palladium(0), palladium(II) acetate, or tris(dibenzylideneacetone)dipalladium(0) and a ligand such as 2-dicyclohexylphosphino-2′,4′,6′-triisopropylbiphenyl can be used. In addition, an inorganic base such as potassium carbonate, sodium carbonate, or tripotassium phosphate or the like can be used. Furthermore, tetrahydrofuran, dioxane, water, or the like can be used as a solvent. Reagents that can be used in the reaction are not limited thereto.

Although an example of a method for synthesizing the organic compound of one embodiment of the present invention is described above, the present invention is not limited thereto and any other synthesis method may be employed.

The structures described in this embodiment can be used in combination with any of the structures described in the other embodiments, as appropriate.

### Embodiment 2

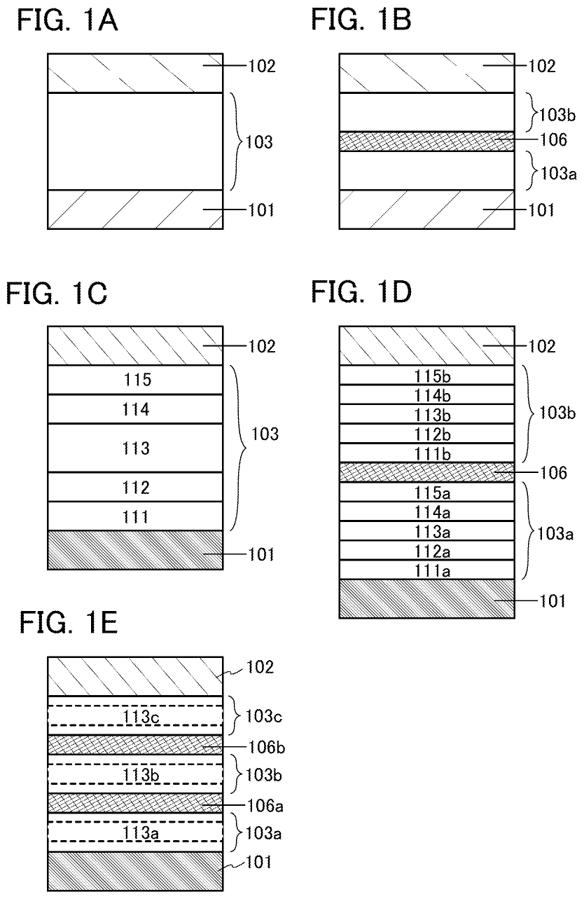

In this embodiment, light-emitting devices including any of the organic compounds described in Embodiment 1 are described with reference to FIGS. 1A to 1E.

### <<Specific Structure of Light-Emitting Device>>

Among light-emitting devices shown in FIGS. 1A to 1E, the light-emitting devices shown in FIGS. 1A and 1C each have a structure in which an EL layer is interposed between a pair of electrodes (a single structure), whereas the light-emitting devices shown in FIGS. 1B, 1D, and 1E each have a structure in which, between a pair of electrodes, two or more EL layers are stacked with a charge-generation layer positioned therebetween (a tandem structure). Note that the structure of the EL layer is common between these structures. When the light-emitting device in FIG. 1D has a microcavity structure, a first electrode 101 is formed as a reflective electrode and a second electrode 102 is formed as a transflective electrode. Thus, a single-layer structure or a stacked-layer structure can be formed using one or more kinds of desired electrode materials. Note that the second electrode 102 is formed after formation of an EL layer 103b, with the use of a material selected as described above.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In each of the light-emitting devices in FIGS. 1A and 1C, when the first electrode 101 is an anode, an EL layer 103 is formed over the first electrode 101 by a vacuum evaporation method. Specifically, as shown in FIG. 1C, a hole-injection layer 111, a hole-transport layer 112, a light-emitting layer 113, an electron-transport layer 114, and an electron-injection layer 115 are sequentially stacked as the EL layer 103 between the first electrode 101 and the second electrode 102 by a vacuum evaporation method. In each of the light-emitting devices in FIGS. 1B, 1D, and 1E, when the first electrode 101 is an anode, a hole-injection layer 111a and a hole-transport layer 112a of an EL layer 103a are sequentially stacked over the first electrode 101 by a vacuum evaporation method. After the EL layer 103a and a charge-generation layer 106 (or a charge-generation layer 106a) are formed, a hole-injection layer 111b and a hole-transport layer 112b of the EL layer 103b are sequentially stacked over the charge-generation layer 106 (or the charge-generation layer 106a) in a similar manner.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain one or both of an organic acceptor material and a material having a high hole-injection property.

The organic acceptor material allows holes to be generated in another organic compound whose highest occupied molecular orbital (HOMO) level is close to the LUMO level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylene-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferred because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 in the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among these oxides, molybdenum oxide is preferable because it is stable in the air, has a low hygroscopic property, and is easily handled. Other examples are phthalocyanine (abbreviation: H₂Pc), a phthalocyanine-based compound such as copper phthalocyanine (abbreviation: CuPc), and the like.

Other examples are aromatic amine compounds, which are low molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples are high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

As the material having a high hole-injection property, a composite material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a composite material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have a hole-transport property higher than an electron-transport property.

As the hole-transport material, materials having a high hole-transport property, such as a π-electron rich heteroaromatic compound (e.g., a carbazole derivative, a furan derivative, or a thiophene derivative) and an aromatic amine (a compound having an aromatic amine skeleton), are preferable.

Examples of the carbazole derivative (a compound having a carbazole skeleton) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (a compound having a furan skeleton) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (a compound having a thiophene skeleton) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiPNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### <Hole-Transport Layer>

The hole-transport layers (112, 112a, and 112b) transport the holes, which are injected from the first electrode 101 by the hole-injection layers (111, 111a, and 111b), to the light-emitting layers (113, 113a, and 113b). Note that the hole-transport layers (112, 112a, and 112b) contain a hole-transport material. Thus, the hole-transport layers (112, 112a, and 112b) can be formed using a hole-transport material that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). Furthermore, a stacked-layer structure in which one light-emitting layer contains two or more kinds of light-emitting substances may be employed.

The light-emitting layers (113, 113a, and 113b) may each contain one or more kinds of organic compounds (e.g., a host material) in addition to a light-emitting substance (guest material).

In the case where a plurality of host materials are used in the light-emitting layers (113, 113a, and 113b), a second host material that is additionally used is preferably a substance having a larger energy gap than a known guest material and a first host material. Preferably, the lowest singlet excitation energy level (S1 level) of the second host material is higher than that of the first host material, and the lowest triplet excitation energy level (T1 level) of the second host material is higher than that of the guest material. Preferably, the lowest triplet excitation energy level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved at the same time.

As an organic compound used as the host material (including the first host material and the second host material), organic compounds such as the hole-transport materials usable in the hole-transport layers (112, 112a, and 112b) and electron-transport materials usable in electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S1 level and the T1 level and functions as a TADF material capable of converting triplet excitation energy into singlet excitation energy. In an example of a preferred combination of two or more kinds of organic compounds forming an exciplex, one of the two or more kinds of organic compounds has a π-electron deficient heteroaromatic ring and the other has a π-electron rich heteroaromatic ring. A phosphorescent substance such as an iridium-, rhodium-, or platinum-based organometallic complex or a metal complex may be used as one component of the combination for forming an exciplex.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that exhibit fluorescence (fluorescent substances) can be given as examples of the light-emitting substance that converts singlet excitation energy into light and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of pyrene derivatives include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine] (abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine] (abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra(tert-butyl)perylene (abbreviation: TBP), N,N″-(2-tert-butylanthracene-9,10-diyldi-4,1-phenylene)bis[N,N′,N″-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light>>

Examples of the light-emitting substance that converts triplet excitation energy into light and can be used in the light-emitting layer 113 include substances that exhibit phosphorescence (phosphorescent materials) and thermally activated delayed fluorescent (TADF) materials that exhibit thermally activated delayed fluorescence.

A phosphorescent substance is a compound that exhibits phosphorescence but does not exhibit fluorescence at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is particularly preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm, Blue or Green)>>

As examples of a phosphorescent substance which emits blue or green light and whose emission spectrum has a peak wavelength of greater than or equal to 450 nm and less than or equal to 570 nm, the following substances can be given.

Examples include organometallic complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm, Green or Yellow)>>

As examples of a phosphorescent substance which emits green or yellow light and whose emission spectrum has a peak wavelength of greater than or equal to 495 nm and less than or equal to 590 nm, the following substances can be given.

Examples of the phosphorescent substance include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), and bis[2-(2-pyridinyl-κN)phenyl-κC][2-(4-methyl-5-phenyl-2-pyridinyl-κN)phenyl-κC]; organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato)(monophenanthroline)terbium(III) (abbreviation: [Tb(acac)₃(Phen)]).

<<Phosphorescent Substance (from 570 nm to 750 nm, Yellow or Red)>>

As examples of a phosphorescent substance which emits yellow or red light and whose emission spectrum has a peak wavelength of greater than or equal to 570 nm and less than or equal to 750 nm, the following substances can be given.

Examples include organometallic complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-(5-(2,6-dimethylphenyl)-3-(3,5-dimethylphenyl)-2-pyrazinyl-κN)-4,6-dimethylphenyl-κC](2,2′,6,6′-tetramethyl-3,5-heptanedionato-κ²O,O′)iridium(III) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials described below can be used as the TADF material. The TADF material is a material that has a small difference between its S1 and T1 levels (preferably less than or equal to 0.2 eV), enables up-conversion of a triplet excited state into a singlet excited state (i.e., reverse intersystem crossing) using a little thermal energy, and efficiently exhibits light (fluorescence) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Note that delayed fluorescence by the TADF material refers to light emission having a spectrum similar to that of normal fluorescence and an extremely long lifetime. The lifetime is longer than or equal to 1×10⁻⁶ seconds, preferably longer than or equal to 1×10⁻³ seconds.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

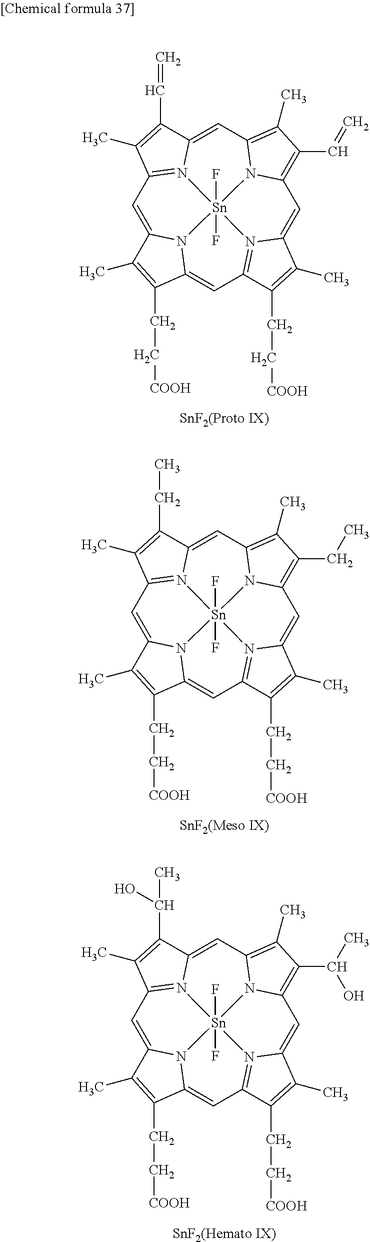

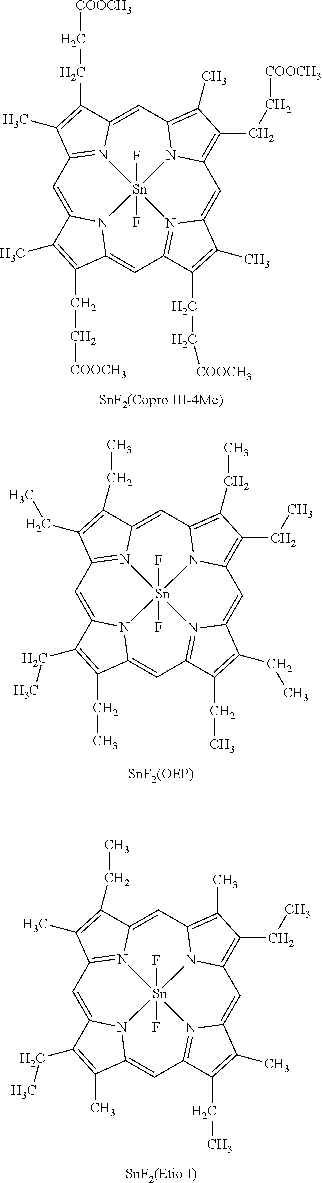

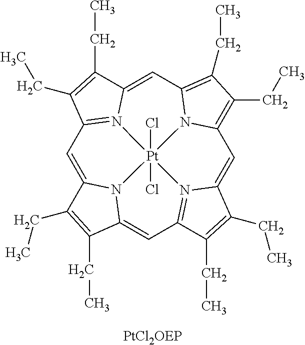

Alternatively, a heterocyclic compound having a π-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic ring is directly bonded to a π-electron deficient heteroaromatic ring is particularly preferable because both the donor property of the π-electron rich heteroaromatic ring and the acceptor property of the π-electron deficient heteroaromatic ring are improved and the energy difference between the singlet excited state and the triplet excited state becomes small.

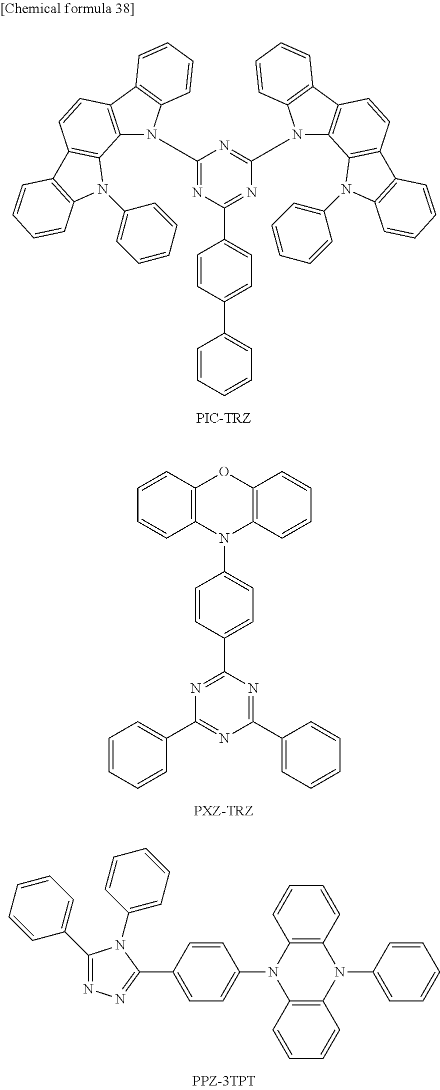 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

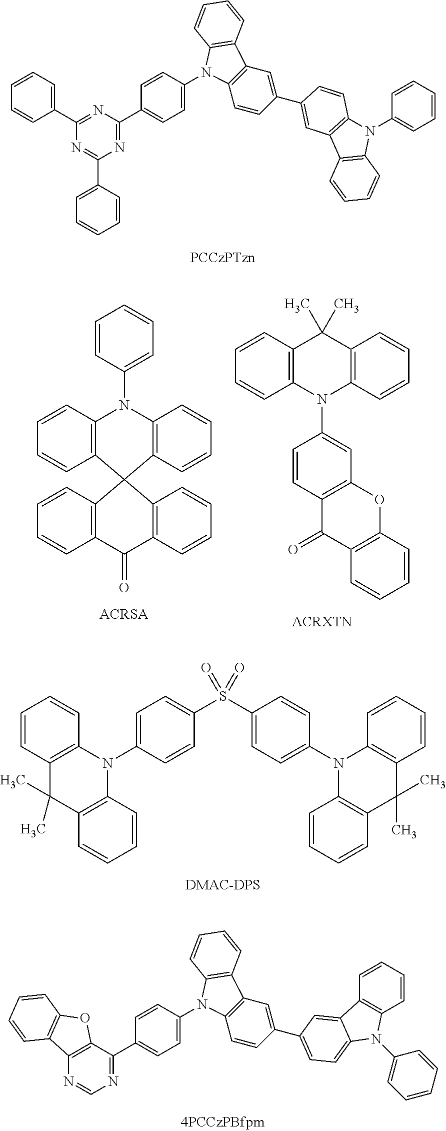

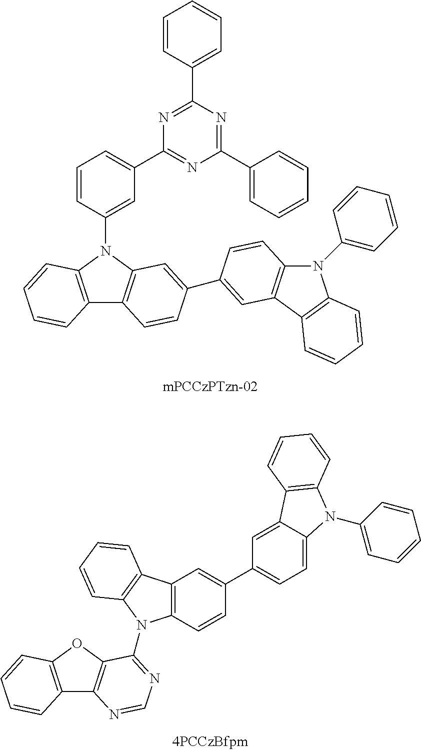 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-n2c3ccccc3c3cc(-c4ccc5c(c4)c4ccccc4n5-c4ncnc5c4oc4ccccc45)ccc32)cc1]

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, 113b, and 113c), one or more kinds selected from substances having a larger energy gap than the light-emitting substance (guest material) are used.

### <<Host Material for Fluorescence>>

In the case where the light-emitting substance used in the light-emitting layers (113, 113a, 113b, and 113c) is a fluorescent substance, an organic compound (a host material) used in combination with the fluorescent substance is preferably an organic compound that has a high energy level in a singlet excited state and has a low energy level in a triplet excited state, or an organic compound having a high fluorescence quantum yield. Therefore, the hole-transport material (described above) and the electron-transport material (described below) shown in this embodiment, for example, can be used as long as they are organic compounds that satisfy such a condition.

In terms of a preferred combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescence>>

In the case where the light-emitting substance used in the light-emitting layers (113, 113a, 113b, and 113c) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferably employed, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compound (the host material and the assist material), some of which overlap the above specific examples, include an aromatic amine, a carbazole derivative, a dibenzothiophene derivative, a dibenzofuran derivative, zinc- and aluminum-based metal complexes, an oxadiazole derivative, a triazole derivative, a benzimidazole derivative, a quinoxaline derivative, a dibenzoquinoxaline derivative, a pyrimidine derivative, a triazine derivative, a pyridine derivative, a bipyridine derivative, and a phenanthroline derivative.

Among the above organic compounds, specific examples of the aromatic amine and the carbazole derivative, which are organic compounds having a high hole-transport property, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Among the above, specific examples of metal complexes that are organic compounds having a high electron-transport property (electron-transport materials) include zinc- and aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline skeleton or a benzoquinoline skeleton. Such metal complexes are preferable as the host material.

Other examples of preferred host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the phenanthroline derivative, and the like, which are organic compounds having a high electron-transport property (electron-transport materials), include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), 4,4′-bis(5-methylbenzoxazol-2-yl)stilbene (abbreviation: BzOS), bathophenanthroline (abbreviation: BPhen), bathocuproine (abbreviation: BCP), 2,9-bis(naphthalen-2-yl)-4,7-diphenyl-1,10-phenanthroline (abbreviation: NBPhen), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), and 6-[3-(dibenzothiophen-4-yl)phenyl]dibenzo[f,h]quinoxaline (abbreviation: 6mDBTPDBq-II). Such derivatives are preferable as the host material.

Among the above, specific examples of a heterocyclic compound having a diazine skeleton, a heterocyclic compound having a triazine skeleton, and a heterocyclic compound having a pyridine skeleton, which are organic compounds having a high electron-transport property (electron-transport materials), include 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), and 1,3,5-tri[3-(3-pyridyl)phenyl]benzene (abbreviation: TmPyPB). Such heterocyclic compounds are preferable as the host material.

Moreover, high molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are preferable as the host material.

Furthermore, for example, 9-phenyl-9′-(4-phenyl-2-quinazolinyl)-3,3′-bi-9H-carbazole (abbreviation: PCCzQz) having bipolar properties, which is an organic compound having a high hole-transport property and a high electron-transport property, can be used as the host material.

### <Electron-Transport Layer>

The electron-transport layers (114, 114a, and 114b) transport the electrons, which are injected from the second electrode 102 and the charge-generation layers (106, 106a, and 106b) by electron-injection layers (115, 115a, and 115b) described later, to the light-emitting layers (113, 113a, and 113b). Note that the electron-transport layers (114, 114a, and 114b) contain an electron-transport material. It is preferable that the electron-transport material used in the electron-transport layers (114, 114a, and 114b) be a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. The organic compound of one embodiment of the present invention is preferably used in the electron-transport layers (114, 114a, and 114b). The electron-transport layers (114, 114a, and 114b) function even with a single-layer structure; however, when the electron-transport layer has a stacked-layer structure including two or more layers as needed, the device characteristics can be improved.

### <<Electron-Transport Material>>

Examples of the electron-transport material that can be used for the electron-transport layers (114, 114a, and 114b) include materials having a high electron-transport property (electron-transport materials), such as an organic compound having a structure where an aromatic ring is fused to a furan ring of a furodiazine skeleton, a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, and a π-electron deficient heteroaromatic compound (e.g., a nitrogen-containing heteroaromatic compound).

Specific examples of the electron-transport material include metal complexes having a quinoline skeleton or a benzoquinoline skeleton, such as 2-[3′-(dibenzothiophen-4-yl)biphenyl-3-yl]dibenzo[f,h]quinoxaline (abbreviation: 2mDBTBPDBq-II), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 4-[3-(dibenzothiophen-4-yl)phenyl]-8-(naphthalen-2-yl)-[1]benzofuro[3,2-d]pyrimidine (abbreviation: 8βN-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 9-[(3′-dibenzothiophen-4-yl)biphenyl-3-yl]naphtho[1′,2′:4,5]furo[2,3-b]pyrazine (abbreviation: 9mDBtBPNfpr), 8-[3′-(dibenzothiophen-4-yl)(1,1′-biphenyl-3-yl)]naphtho[1′,2′:4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm), 8-(1,1′-biphenyl-4-yl)-4-[3-(dibenzothiophen-4-yl)phenyl]-[1]benzofuro[3,2-d]pyrimidine (abbreviation: 8BP-4mDBtPBfpm), tris(8-quinolinolato)aluminum(III) (abbreviation: Alq₃), Almq₃, BeBq₂, bis(2-methyl-8-quinolinolato)(4-phenylphenolato)aluminum(III) (abbreviation: BAlq), and bis(8-quinolinolato)zinc(II) (abbreviation: Znq), and metal complexes having an oxazole skeleton or a thiazole skeleton, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Other than the metal complexes, oxadiazole derivatives such as PBD, OXD-7, and CO11, triazole derivatives such as TAZ and p-EtTAZ, imidazole derivatives (including benzimidazole derivatives) such as TPBI and mDBTBIm-II, an oxazole derivative such as BzOS, phenanthroline derivatives such as BPhen, BCP, and NBPhen, quinoxaline derivatives and dibenzoquinoxaline derivatives such as 2mDBTPDBq-II, 2mDBTBPDBq-II, 2mCzBPDBq, 2CzPDBq-III, 7mDBTPDBq-II, and 6mDBTPDBq-II, pyridine derivatives such as 35DCzPPy and TmPyPB, pyrimidine derivatives such as 4,6mPnP2Pm, 4,6mDBTP2Pm-II, and 4,6mCzP2Pm, and triazine derivatives such as PCCzPTzn and mPCCzPTzn-02 can be used as the electron-transport material.

High-molecular compounds such as poly(2,5-pyridinediyl) (abbreviation: PPy), poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, and 114b) is not limited to a single layer and may be a stack of two or more layers each containing any of the above substances.

### <Electron-Injection Layer>

The electron-injection layers (115, 115a, and 115b) contain a substance having a high electron-injection property. The electron-injection layers (115, 115a, and 115b) are layers for increasing the efficiency of electron injection from the second electrode 102 and are preferably formed using a material whose value of the LUMO level has a small difference (0.5 eV or less) from the work function of a material used for the second electrode 102. Thus, the electron-injection layers (115, 115a, and 115b) can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal and a compound thereof such as ytterbium (Yb) and erbium fluoride (ErF₃) can also be used. Electride may also be used for the electron-injection layers (115, 115a, and 115b). Examples of the electride include a substance in which electrons are added at high concentration to calcium oxide-aluminum oxide. Any of the substances used for the electron-transport layers (114, 114a, and 114b), which are given above, can also be used.

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A composite material in which an organic compound and a metal are mixed may also be used for the electron-injection layers (115, 115a, and 115b). The organic compound used here preferably has a LUMO level higher than or equal to −3.6 eV and lower than or equal to −2.3 eV. Moreover, a material having an unshared electron pair is preferable.

Therefore, the above organic compound is preferably a material having an unshared electron pair, such as a heterocyclic compound having a pyridine skeleton, a diazine skeleton (e.g., a pyrimidine skeleton or a pyrazine skeleton), or a triazine skeleton.

Examples of the heterocyclic compound having a pyridine skeleton include 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathocuproine (abbreviation: BCP), 2,9-bis(naphthalen-2-yl)-4,7-diphenyl-1,10-phenanthroline (abbreviation: NBPhen), and bathophenanthroline (abbreviation: BPhen).

Examples of the heterocyclic compound having a diazine skeleton include 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), and 4-{3-[3′-(9H-carbazol-9-yl)]biphenyl-3-yl}benzofuro[3,2-d]pyrimidine (abbreviation: 4mCzBPBfpm).

Examples of the heterocyclic compound having a triazine skeleton include 2-{4-[3-(N-phenyl-9H-carbazol-3-yl)-9H-carbazol-9-yl]phenyl}-4,6-diphenyl-1,3,5-triazine (abbreviation: PCCzPTzn), 2,4,6-tris[3′-(pyridin-3-yl)biphenyl-3-yl]-1,3,5-triazine (abbreviation: TmPPPyTz), and 2,4,6-tris(2-pyridyl)-1,3,5-triazine (abbreviation: 2Py3 Tz).

As a metal, a transition metal that belongs to Group 5, Group 7, Group 9, or Group 11 or a material that belongs to Group 13 in the periodic table is preferably used, and examples include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

When the charge-generation layer 106 is provided between the two EL layers (103a and 103b) as in the light-emitting device in FIG. 1D, a structure in which a plurality of EL layers are stacked between the pair of electrodes (the structure is also referred to as a tandem structure) can be obtained.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples are vanadium oxide, niobium oxide, tantalum oxide, chromium oxide, molybdenum oxide, tungsten oxide, manganese oxide, and rhenium oxide. Any of the above-described acceptor materials may be used. Furthermore, a p-type layer may be a mixed film obtained by mixing a hole-transport material and an electron acceptor, or a stack of a film containing a hole-transport material and a film containing an electron acceptor.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. A specific value of the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 1D illustrates the structure in which two EL layers 103 are stacked, three or more EL layers may be stacked with charge-generation layers each provided between two adjacent EL layers. FIG. 1E illustrates a structure in which three EL layers (the EL layer 103a, the EL layer 103b, and an EL layer 103c) are stacked with two charge-generation layers (the charge-generation layer 106a and the charge-generation layer 106b) positioned therebetween.

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as acrylic, polypropylene, polyester, polyvinyl fluoride, polyvinyl chloride, polyamide, polyimide, aramid, epoxy, an inorganic vapor deposition film, and paper.

For fabrication of the light-emitting device in this embodiment, a vacuum process such as an evaporation method or a solution process such as a spin coating method and an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), and the like can be used. Specifically, the layers having various functions (the hole-injection layers (111, 111a, and 111b), the hole-transport layers (112, 112a, and 112b), the light-emitting layers (113, 113a, 113b, and 113c), the electron-transport layers (114, 114a, and 114b), the electron-injection layers (115, 115a, and 115b)) included in the EL layers and the charge-generation layers (106, 106a, and 106b) of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high molecular compound (e.g., an oligomer, a dendrimer, or a polymer), a middle molecular compound (a compound between a low molecular compound and a high molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Note that materials that can be used for the layers (the hole-injection layers (111, 111a, and 111b), the hole-transport layers (112, 112a, and 112b), the light-emitting layers (113, 113a, 113b, and 113c), the electron-transport layers (114, 114a, and 114b), and the electron-injection layers (115, 115a, and 115b)) included in the EL layers (103, 103a, and 103b) and the charge-generation layers (106, 106a, and 106b) of the light-emitting device described in this embodiment are not limited to the materials described in this embodiment, and other materials can be used in combination as long as the functions of the layers are fulfilled.

The structures described in this embodiment can be used in combination with any of the structures described in the other embodiments as appropriate.

### Embodiment 3

In this embodiment, specific structure examples and manufacturing methods of a light-emitting apparatus (also referred to as a display panel) of one embodiment of the present invention will be described.

### <Structure Example 1 of Light-Emitting Apparatus 700>

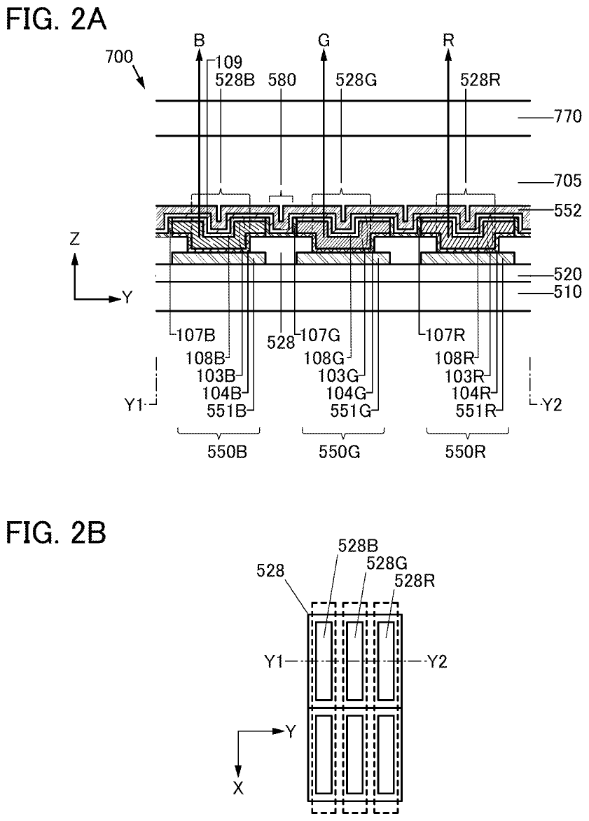

A light-emitting apparatus 700 illustrated in FIG. 2A includes a light-emitting device 550B, a light-emitting device 550G, a light-emitting device 550R, and a partition 528. The light-emitting device 550B, the light-emitting device 550G, the light-emitting device 550R, and the partition 528 are formed over a functional layer 520 provided over a first substrate 510. The functional layer 520 includes, for example, a driver circuit GD, a driver circuit SD, pixel circuits, and the like that are composed of a plurality of transistors, and wirings that electrically connect these circuits. Note that these driver circuits are electrically connected to the light-emitting device 550B, the light-emitting device 550G, and the light-emitting device 550R, for example, to drive them. The light-emitting apparatus 700 includes an insulating layer 705 over the functional layer 520 and the light-emitting devices, and the insulating layer 705 has a function of attaching a second substrate 770 and the functional layer 520. The driver circuit GD and the driver circuit SD will be described in Embodiment 4.

The light-emitting device 550B, the light-emitting device 550G, and the light-emitting device 550R each have the device structure described in Embodiment 2. Specifically, the case is described in which the EL layer 103 in the structure illustrated in FIG. 1A differs between the light-emitting devices.

In this specification and the like, a structure in which light-emitting layers in light-emitting devices of different colors (for example, blue (B), green (G), and red (R)) are separately formed or separately patterned may be referred to as a side-by-side (SBS) structure.

The light-emitting device 550B includes an electrode 551B, an electrode 552, an EL layer 103B, and an insulating layer 107B. Note that a specific structure of each layer is as described in Embodiment 2. The EL layer 103B has a stacked-layer structure of layers having different functions including a light-emitting layer. Although FIG. 2A illustrates only a hole-injection/transport layer 104B, an electron-transport layer 108B, and an electron-injection layer 109 as layers of the EL layer 103B, which includes the light-emitting layer, the present invention is not limited thereto. Note that the hole-injection/transport layer 104B represents the layer having the functions of the hole-injection layer and the hole-transport layer described in Embodiment 2 and may have a stacked-layer structure. Note that in this specification, a hole-injection/transport layer in any light-emitting device can be interpreted in the above manner. The electron-transport layer 108B may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, the insulating layer 107B is formed while a resist formed over some layers of the EL layer 103B (in this embodiment, the layers up to the electron-transport layer 108B over the light-emitting layer) remains over the electrode 551B. Thus, the insulating layer 107B is formed in contact with side surfaces (or end portions) of the above layers in the EL layer 103B. Accordingly, entry of oxygen, moisture, or constituent elements thereof through the side surface of the EL layer 103B into the inside of the EL layer 103B can be inhibited. For the insulating layer 107B, aluminum oxide, magnesium oxide, hafnium oxide, gallium oxide, indium gallium zinc oxide, silicon nitride, or silicon nitride oxide can be used, for example. The insulating layer 107B can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103B (the layers up to the electron-transport layer 108B) and the insulating layer 107B. The electron-injection layer 109 preferably has a stacked-layer structure of two or more layers having different electric resistances. For example, the electron-injection layer 109 may have one of the following structures: a structure in which a first layer in contact with the electron-transport layer 108B is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108B.

The electrode 552 is formed over the electron-injection layer 109. Note that the electrode 551B and the electrode 552 have an overlap region. The EL layer 103B is positioned between the electrode 551B and the electrode 552. Thus, the electron-injection layer 109 is positioned at the side surfaces (or end portions) of some layers of the EL layer 103B with the insulating layer 107B therebetween, or the electrode 552 is positioned at the side surfaces (or end portions) of some layers of the EL layer 103B with the electron-injection layer 109 and the insulating layer 107B therebetween. Hence, the EL layer 103B and the electrode 552, specifically the hole-injection/transport layer 104B in the EL layer 103B and the electrode 552 can be prevented from being electrically short-circuited.

The EL layer 103B illustrated in FIG. 2A has the same structure as the EL layers 103, 103a, 103b, and 103c described in Embodiment 2. The EL layer 103B is capable of emitting blue light, for example.

The light-emitting device 550G includes an electrode 551G, the electrode 552, an EL layer 103G, and an insulating layer 107G. Note that a specific structure of each layer is as described in Embodiment 2. The EL layer 103G has a stacked-layer structure of layers having different functions including a light-emitting layer. Although FIG. 2A illustrates only a hole-injection/transport layer 104G, an electron-transport layer 108G, and the electron-injection layer 109 as layers of the EL layer 103G, which includes the light-emitting layer, the present invention is not limited thereto. Note that the hole-injection/transport layer 104G represents the layer having the functions of the hole-injection layer and the hole-transport layer described in Embodiment 2 and may have a stacked-layer structure.

The electron-transport layer 108G may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, the insulating layer 107G is formed while a resist formed over some layers of the EL layer 103G (in this embodiment, the layers up to the electron-transport layer 108G over the light-emitting layer) remains over the electrode 551G. Thus, the insulating layer 107G is formed in contact with side surfaces (or end portions) of the above layers in the EL layer 103G. Accordingly, entry of oxygen, moisture, or constituent elements thereof through the side surface of the EL layer 103G into the inside of the EL layer 103G can be inhibited. For the insulating layer 107G, aluminum oxide, magnesium oxide, hafnium oxide, gallium oxide, indium gallium zinc oxide, silicon nitride, or silicon nitride oxide can be used, for example. The insulating layer 107G can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103G (the layers up to the electron-transport layer 108G) and the insulating layer 107G. The electron-injection layer 109 preferably has a stacked-layer structure of two or more layers having different electric resistances. For example, the electron-injection layer 109 may have one of the following structures: a structure in which a first layer in contact with the electron-transport layer 108G is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108G.

The electrode 552 is formed over the electron-injection layer 109. Note that the electrode 551G and the electrode 552 have an overlap region. The EL layer 103G is positioned between the electrode 551G and the electrode 552. Thus, the electron-injection layer 109 is positioned at the side surfaces (or end portions) of some layers of the EL layer 103G with the insulating layer 107G therebetween, or the electrode 552 is positioned at the side surfaces (or end portions) of some layers of the EL layer 103G with the electron-injection layer 109 and the insulating layer 107G therebetween. Hence, the EL layer 103G and the electrode 552, specifically the hole-injection/transport layer 104G in the EL layer 103G and the electrode 552 can be prevented from being electrically short-circuited.

The EL layer 103G illustrated in FIG. 2A has the same structure as the EL layers 103, 103a, 103b, and 103c described in Embodiment 2. The EL layer 103G is capable of emitting green light, for example.

The light-emitting device 550R includes an electrode 551R, the electrode 552, an EL layer 103R, and an insulating layer 107R. Note that a specific structure of each layer is as described in Embodiment 2. The EL layer 103R has a stacked-layer structure of layers having different functions including a light-emitting layer. Although FIG. 2A illustrates only a hole-injection/transport layer 104R, an electron-transport layer 108R, and the electron-injection layer 109 as layers of the EL layer 103R, which includes the light-emitting layer, the present invention is not limited thereto. The hole-injection/transport layer 104R represents the layer having the functions of the hole-injection layer and the hole-transport layer described in Embodiment 2 and may have a stacked-layer structure. Note that in this specification, a hole-injection/transport layer in any light-emitting device can be interpreted in the above manner. The electron-transport layer 108R may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, the insulating layer 107R is formed while a resist formed over some layers of the EL layer 103R (in this embodiment, the layers up to the electron-transport layer 108R over the light-emitting layer) remains over the electrode 551R. Thus, the insulating layer 107R is formed in contact with side surfaces (or end portions) of the above layers in the EL layer 103R. Accordingly, entry of oxygen, moisture, or constituent elements thereof through the side surface of the EL layer 103R into the inside of the EL layer 103R can be inhibited. For the insulating layer 107R, aluminum oxide, magnesium oxide, hafnium oxide, gallium oxide, indium gallium zinc oxide, silicon nitride, or silicon nitride oxide can be used, for example. The insulating layer 107R can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103R (the layers up to the electron-transport layer 108R) and the insulating layer 107R. The electron-injection layer 109 preferably has a stacked-layer structure of two or more layers having different electric resistances. For example, the electron-injection layer 109 may have one of the following structures: a structure in which a first layer in contact with the electron-transport layer 108R is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108R.

The electrode 552 is formed over the electron-injection layer 109. Note that the electrode 551R and the electrode 552 have an overlap region. The EL layer 103R is positioned between the electrode 551R and the electrode 552. Thus, the electron-injection layer 109 is positioned at the side surfaces (or end portions) of some layers of the EL layer 103R with the insulating layer 107R therebetween, or the electrode 552 is positioned at the side surfaces (or end portions) of some layers of the EL layer 103R with the electron-injection layer 109 and the insulating layer 107R therebetween. Hence, the EL layer 103R and the electrode 552, specifically the hole-injection/transport layer 104R in the EL layer 103R and the electrode 552 can be prevented from being electrically short-circuited.

The EL layer 103R illustrated in FIG. 2A has the same structure as the EL layers 103, 103a, 103b, and 103c described in Embodiment 2. The EL layer 103R is capable of emitting red light, for example.

A space 580 is provided between the EL layer 103B, the EL layer 103G, and the EL layer 103R. In each of the EL layers, particularly the hole-injection layer, which is included in the hole-transport region between the anode and the light-emitting layer, often has high conductivity; therefore, a hole-injection layer formed as a layer shared by adjacent light-emitting devices might cause crosstalk. Thus, providing the space 580 between the EL layers as shown in this structure example can suppress occurrence of crosstalk between adjacent light-emitting devices.

When electrical continuity is established between the EL layer 103B, the EL layer 103G, and the EL layer 103R in a light-emitting apparatus (display panel) with a high resolution exceeding 1000 ppi, crosstalk occurs, resulting in a narrower color gamut that the light-emitting apparatus is capable of reproducing. Providing the space 580 in a high-resolution display panel with more than 1000 ppi, preferably more than 2000 ppi, or further preferably in an ultrahigh-resolution display panel with more than 5000 ppi allows the display panel to express vivid colors.

As illustrated in FIG. 2B, the partition 528 has an opening 528B, an opening 528G, and an opening 528R. As illustrated in FIG. 2A, the opening 528B overlaps the electrode 551B, the opening 528G overlaps the electrode 551G, and the opening 528R overlaps the electrode 551R. Note that a cross-sectional view taken along the dashed-dotted line Y1-Y2 in FIG. 2B corresponds to a schematic cross-sectional view of the light-emitting apparatus illustrated in FIG. 2A.

The EL layers 103B, 103G, and 103R are processed to be separated by patterning using a photolithography method; hence, a high-resolution light-emitting apparatus (display panel) can be fabricated. End portions (side surfaces) of the EL layer (the hole-injection/transport layer, the light-emitting layer, and the electron-transport layer) processed by patterning using a photolithography method have substantially one surface (or are positioned on substantially the same plane). In this case, the space 580 between the EL layers is preferably 5 μm or less, further preferably 1 μm or less.

In the EL layer, particularly the hole-injection layer, which is included in the hole-transport region between the anode and the light-emitting layer, often has high conductivity; therefore, a hole-injection layer formed as a layer shared by adjacent light-emitting devices might cause crosstalk. Thus, processing the EL layers to be separated by patterning using a photolithography method as shown in this structure example can suppress occurrence of crosstalk between adjacent light-emitting devices.

In this specification and the like, a device formed using a metal mask or a fine metal mask (FMM) may be referred to as a device having a metal mask (MM) structure. In this specification and the like, a device formed without using a metal mask or an FMM may be referred to as a device having a metal maskless (MML) structure.

### <Example 1 of Method for Manufacturing Light-Emitting Apparatus>

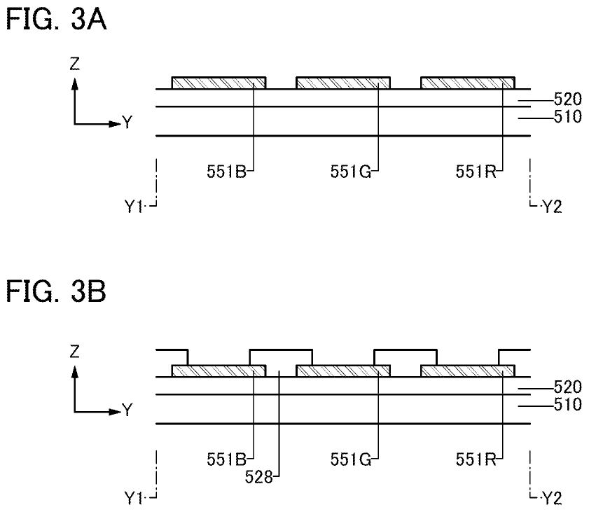

The electrode 551B, the electrode 551G, and the electrode 551R are formed as illustrated in FIG. 3A. For example, a conductive film is formed over the functional layer 520 over the first substrate 510 and processed into predetermined shapes by a photolithography method.

The conductive film can be formed by any of a sputtering method, a chemical vapor deposition (CVD) method, a vacuum evaporation method, a pulsed laser deposition (PLD) method, an atomic layer deposition (ALD) method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical processing methods using a photolithography method. In one of the methods, a resist mask is formed over a thin film that is to be processed, the thin film is processed by etching or the like, and then the resist mask is removed. In the other method, a photosensitive thin film is formed and then processed into a desired shape by light exposure and development.

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when exposure is performed by scanning with a beam such as an electron beam.

For etching of a thin film using a resist mask, a dry etching method, a wet etching method, a sandblast method, or the like can be used.

Next, as illustrated in FIG. 3B, the partition 528 is formed between the electrode 551B, the electrode 551G, and the electrode 551R. For example, the partition 528 can be formed in such a manner that an insulating film covering the electrode 551B, the electrode 551G, and the electrode 551R is formed, and openings are formed by a photolithography method to partly expose the electrode 551B, the electrode 551G, and the electrode 551R. Examples of a material that can be used for the partition 528 include an inorganic material, an organic material, and a composite material of an inorganic material and an organic material. Specifically, it is possible to use an inorganic oxide film, an inorganic nitride film, an inorganic oxynitride film, or the like, or a layered material in which two or more films selected from the above are stacked. More specifically, it is possible to use a silicon oxide film, a film containing acrylic, a film containing polyimide, or the like, or a layered material in which two or more films selected from the above are stacked.

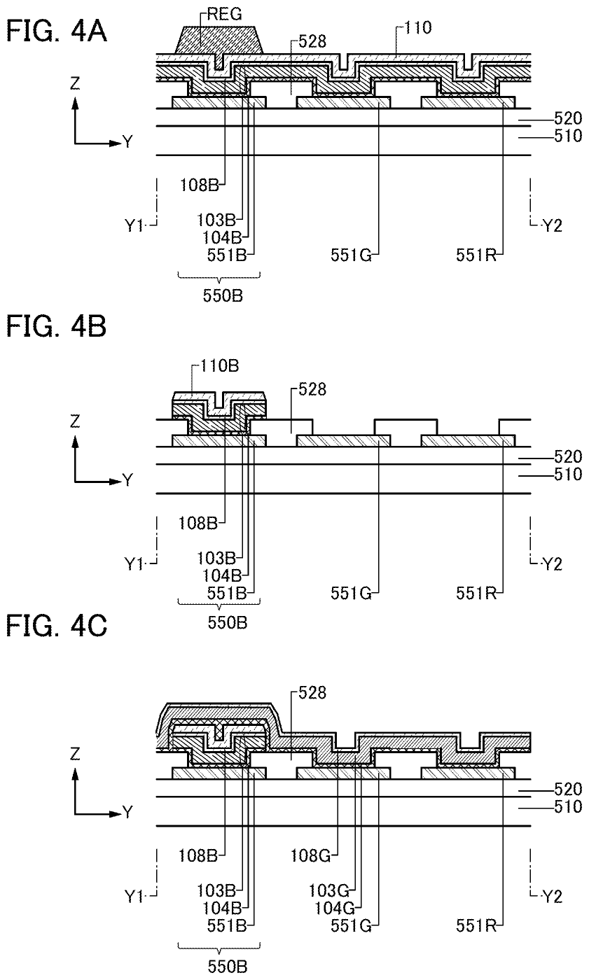

Then, as illustrated in FIG. 4A, the EL layer 103B is formed over the electrode 551B, the electrode 551G, the electrode 551R, and the partition 528. Note that in the EL layer 103B in FIG. 4A, the hole-injection/transport layer 104B, the light-emitting layer, and the electron-transport layer 108B are formed. For example, the EL layer 103B is formed by a vacuum evaporation method over the electrode 551B, the electrode 551G, the electrode 551R, and the partition 528 so as to cover them. Furthermore, a sacrifice layer 110 is formed over the EL layer 103B.

For the sacrifice layer 110, a film highly resistant to etching treatment performed on the EL layer 103B, i.e., a film having high etching selectivity with respect to the EL layer 103B, can be used. The sacrifice layer 110 preferably has a stacked-layer structure of a first sacrifice layer and a second sacrifice layer which have different etching selectivities to the EL layer 103B. For the sacrifice layer 110, it is possible to use a film that can be removed by a wet etching method, which causes less damage to the EL layer 103B. In wet etching, oxalic acid or the like can be used as an etching material. Note that in this specification and the like, a sacrifice layer may be called a mask layer.

For the sacrifice layer 110, an inorganic film such as a metal film, an alloy film, a metal oxide film, a semiconductor film, or an inorganic insulating film can be used, for example. The sacrifice layer 110 can be formed by any of a variety of film formation methods such as a sputtering method, an evaporation method, a CVD method, and an ALD method.

For the sacrifice layer 110, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrifice layer 110. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Indium tin oxide containing silicon, or the like can also be used.

An element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrifice layer 110, an inorganic insulating material such as aluminum oxide, hafnium oxide, or silicon oxide can be used.

The sacrifice layer 110 is preferably formed using a material that can be dissolved in a solvent chemically stable with respect to at least the uppermost film (the electron-transport layer 108B) of the EL layer 103B. Specifically, a material that will be dissolved in water or alcohol can be suitably used for the sacrifice layer 110. In formation of the sacrifice layer 110, it is preferable that application of such a material dissolved in a solvent such as water or alcohol be performed by a wet process and followed by heat treatment for evaporating the solvent. At this time, the heat treatment is preferably performed under a reduced-pressure atmosphere, in which case the solvent can be removed at a low temperature in a short time and thermal damage to the EL layer 103B can be accordingly minimized.

In the case where the sacrifice layer 110 having a stacked-layer structure is formed, the stacked-layer structure can include the first sacrifice layer formed using any of the above-described materials and the second sacrifice layer thereover.

The second sacrifice layer in that case is a film used as a hard mask for etching of the first sacrifice layer. In processing the second sacrifice layer, the first sacrifice layer is exposed. Thus, a combination of films having greatly different etching rates is selected for the first sacrifice layer and the second sacrifice layer. Thus, a film that can be used for the second sacrifice layer can be selected in accordance with the etching conditions of the first sacrifice layer and those of the second sacrifice layer.

For example, in the case where the second sacrifice layer is etched by dry etching involving a fluorine-containing gas (also referred to as fluorine-based gas), the second sacrifice layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a base film which enables the second sacrifice layer to have a high etching selectivity with respect to the base film (i.e., a base film with a low etching rate) in the dry etching involving the fluorine-based gas, and can be used for the first sacrifice layer.

Note that the material for the second sacrifice layer is not limited to the above and can be selected from a variety of materials in view of the etching conditions of the first sacrifice layer and those of the second sacrifice layer. For example, any of the films that can be used for the first sacrifice layer can be used for the second sacrifice layer.

For the second sacrifice layer, for example, a nitride film can be used. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrifice layer. Typically, it is possible to use a film of an oxide or an oxynitride such as silicon oxide, silicon oxynitride, aluminum oxide, aluminum oxynitride, hafnium oxide, or hafnium oxynitride.

Then, the EL layer 103B over the electrode 551B is processed to have a predetermined shape as illustrated in FIG. 4B. For example, a sacrifice layer 110B is formed over the EL layer 103B; a resist is formed to have a desired shape over the sacrifice layer 110B by a photolithography method as a resist mask REG (see FIG. 4A); part of the sacrifice layer 110B not covered with the resist mask REG is removed by etching; the resist mask REG is removed; and part of the EL layer 103B not covered with the sacrifice layer 110B is then removed by etching, i.e., the EL layer 103B over the electrode 551G and the EL layer 103B over the electrode 551R are removed by etching, so that the EL layer 103B over the electrode 551B is processed to have side surfaces (or have their side surfaces exposed) or have a belt-like shape that extends in the direction intersecting the sheet of the diagram. Specifically, dry etching is performed using the sacrifice layer 110B formed in a pattern over the EL layer 103B overlapping the electrode 551B (see FIG. 4B). Note that in the case where the sacrifice layer 110B has the aforementioned stacked-layer structure of the first sacrifice layer and the second sacrifice layer, the EL layer 103B may be processed into a predetermined shape in the following manner: part of the second sacrifice layer is etched with the use of the resist mask REG, the resist mask REG is then removed, and part of the first sacrifice layer is etched with the use of the second sacrifice layer as a mask. The partition 528 can be used as an etching stopper.

Next, as illustrated in FIG. 4C, with the sacrifice layer 110B remaining, the EL layer 103G (including the hole-injection/transport layer 104G, the light-emitting layer, and the electron-transport layer 108G) is formed over the sacrifice layer 110B, the electrode 551G, the electrode 551R, and the partition 528. For example, the EL layer 103G is formed by a vacuum evaporation method over the electrode 551G, the electrode 551R, and the partition 528 so as to cover them.

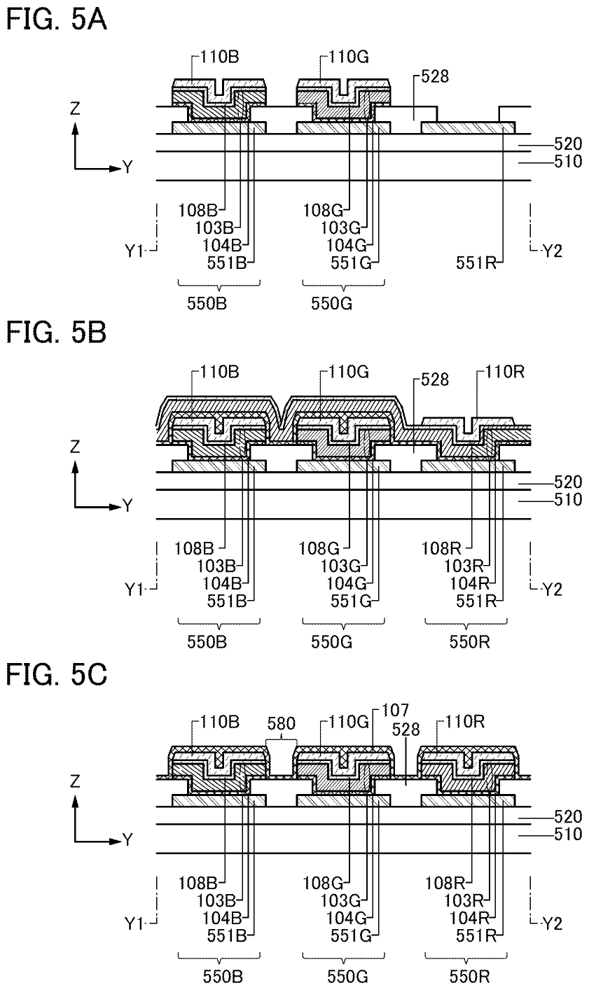

Then, the EL layer 103G over the electrode 551G is processed to have a predetermined shape as illustrated in FIG. 5A. For example, a sacrifice layer 110G is formed over the EL layer 103G; a resist is formed to have a desired shape over the sacrifice layer 110G by a photolithography method as a resist mask; part of the sacrifice layer 110G not covered with the resist mask is removed by etching; the resist mask is removed; and part of the EL layer 103G not covered with the sacrifice layer 110G is then removed by etching, i.e., the EL layer 103G over the electrode 551B and the EL layer 103G over the electrode 551R are removed by etching, so that the EL layer 103G over the electrode 551G is processed to have side surfaces (or have their side surfaces exposed) or have a belt-like shape that extends in the direction intersecting the sheet of the diagram. Specifically, dry etching is performed using the sacrifice layer 110G formed in a pattern over the EL layer 103G overlapping the electrode 551G. Note that in the case where the sacrifice layer 110G has the aforementioned stacked-layer structure of the first sacrifice layer and the second sacrifice layer, the EL layer 103G may be processed into a predetermined shape in the following manner: part of the second sacrifice layer is etched with the use of the resist mask, the resist mask is then removed, and part of the first sacrifice layer is etched with the use of the second sacrifice layer as a mask. The partition 528 can be used as an etching stopper.

Next, as illustrated in FIG. 5B, with the sacrifice layer 110B and the sacrifice layer 110G respectively over the electron-transport layer 108B and the electron-transport layer 108G remaining, the EL layer 103R (including the hole-injection/transport layer 104R, the light-emitting layer, and the electron-transport layer 108R) is formed over the sacrifice layer 110B, the sacrifice layer 110G, the electrode 551R, and the partition 528. For example, the EL layer 103R is formed by a vacuum evaporation method over the sacrifice layer 110B, the sacrifice layer 110G, the electrode 551R, and the partition 528 so as to cover them.

Then, the EL layer 103R over the electrode 551R is processed to have a predetermined shape as illustrated in FIG. 5C. For example, a sacrifice layer 110R is formed over the EL layer 103R; a resist is formed to have a desired shape over the sacrifice layer 110R by a photolithography method as a resist mask; part of the sacrifice layer 110R not covered with the resist mask is removed by etching; the resist mask is removed; and part of the EL layer 103R not covered with the sacrifice layer 110R is then removed by etching, i.e., the EL layer 103R over the electrode 551B and the EL layer 103R over the electrode 551G are removed by etching, so that the EL layer 103R over the electrode 551R is processed to have side surfaces (or have their side surfaces exposed) or have a belt-like shape that extends in the direction intersecting the sheet of the diagram. Specifically, dry etching is performed using the sacrifice layer 110R formed in a pattern over the EL layer 103R overlapping the electrode 551R. Note that in the case where the sacrifice layer 110R has the aforementioned stacked-layer structure of the first sacrifice layer and the second sacrifice layer, the EL layer 103R may be processed into a predetermined shape in the following manner: part of the second sacrifice layer is etched with the use of the resist mask, the resist mask is removed, and part of the first sacrifice layer is etched with the use of the second sacrifice layer as a mask. The partition 528 can be used as an etching stopper.

Then, an insulating layer 107 is formed over the sacrifice layers (110B, 110G, and 110R), the EL layers (103B, 103G, and 103R), and the partition 528. For example, the insulating layer 107 is formed by an ALD method over the sacrifice layers (110, 110G, and 110R), the EL layers (103B, 103G, and 103R), and the partition 528 so as to cover them. In this case, the insulating layer 107 is formed in contact with the side surfaces of the EL layers (103B, 103G, and 103R) as illustrated in FIG. 5C. This can inhibit entry of oxygen, moisture, or constituent elements thereof into the inside through the side surfaces of the EL layers (103B, 103G, and 103R). Examples of the material used for the insulating layer 107 include aluminum oxide, magnesium oxide, hafnium oxide, gallium oxide, indium gallium zinc oxide, silicon nitride, and silicon nitride oxide.

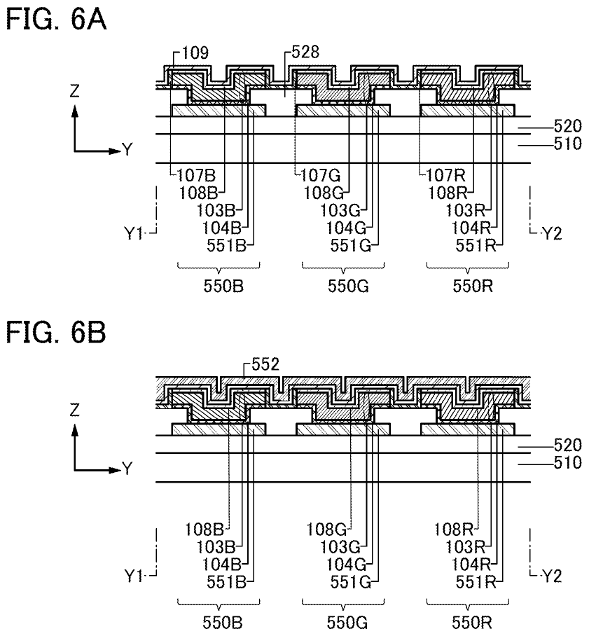

Then, as illustrated in FIG. 6A, the sacrifice layers (110B, 110G, and 110R) and part of the insulating layer 107 are removed, and the electron-injection layer 109 is formed over the insulating layers (107B, 107G, and 107R) and the electron-transport layers (108B, 108G, and 108R). The electron-injection layer 109 is formed by a vacuum evaporation method, for example. The electron-injection layer 109 is positioned at the side surfaces of some layers of the EL layers (103B, 103G, and 103R) (including the hole-injection/transport layers (104R, 104G, and 104B), the light-emitting layers, and the electron-transport layers (108B, 108G, and 108R)) with the insulating layers (107B, 107G, and 107R) therebetween.

Next, as illustrated in FIG. 6B, the electrode 552 is formed. The electrode 552 is formed by a vacuum evaporation method, for example. The electrode 552 is formed over the electron-injection layer 109. The electrode 552 is positioned at the side surfaces (or end portions) of some layers of the EL layers (103B, 103G, and 103R) (including the hole-injection/transport layers (104R, 104G, and 104B), the light-emitting layers, and the electron-transport layers (108B, 108G, and 108R)) with the electron-injection layer 109 and the insulating layers (107B, 107G, and 107R) therebetween. Thus, the EL layers (103B, 103G, and 103R) and the electrode 552, specifically the hole-injection/transport layers (104B, 104G, and 104R) in the EL layers (103B, 103G, and 103R) and the electrode 552 can be prevented from being electrically short-circuited.

Through the above steps, the EL layer 103B, the EL layer 103G, and the EL layer 103R in the light-emitting device 550B, the light-emitting device 550G, and the light-emitting device 550R can be processed to be separated from each other.

The EL layers 103B, 103G, and 103R are processed to be separated by patterning using a photolithography method; hence, a high-resolution light-emitting apparatus (display panel) can be fabricated. End portions (side surfaces) of the EL layer processed by patterning using a photolithography method have substantially one surface (or are positioned on substantially the same plane).

In the EL layer, particularly the hole-injection layer, which is included in the hole-transport region between the anode and the light-emitting layer, often has high conductivity; therefore, a hole-injection layer formed as a layer shared by adjacent light-emitting devices might cause crosstalk. Thus, processing the EL layers to be separated by patterning using a photolithography method as shown in this structure example can suppress occurrence of crosstalk between adjacent light-emitting devices.

### <Structure Example 2 of Light-Emitting Apparatus 700>

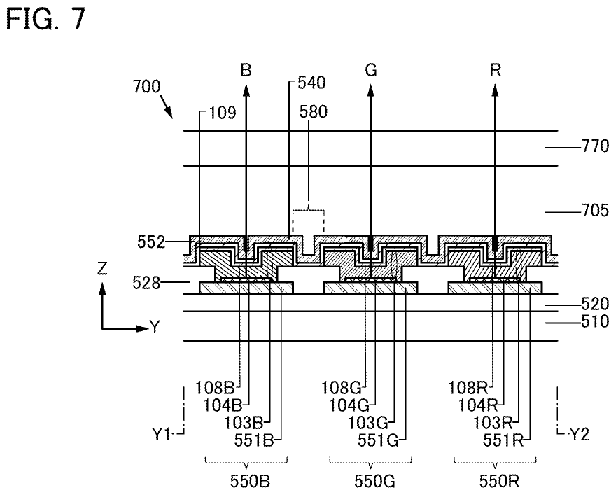

The light-emitting apparatus 700 illustrated in FIG. 7 includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, the driver circuit GD, the driver circuit SD, and the like that are composed of a plurality of transistors, and wirings that electrically connect these circuits. Note that these driver circuits are electrically connected to the light-emitting device 550B, the light-emitting device 550G, and the light-emitting device 550R, for example, to drive them. The driver circuit GD and the driver circuit SD will be described in Embodiment 4.

The light-emitting device 550B, the light-emitting device 550G, and the light-emitting device 550R each have the device structure described in Embodiment 2. Specifically, the case is described in which the EL layer 103 in the structure illustrated in FIG. 1A differs between the light-emitting devices.

Note that specific structures of the light-emitting devices illustrated in FIG. 7 are the same as the structures of the light-emitting devices 550B, 550G, and 550R described with reference to FIGS. 2A and 2B.

As illustrated in FIG. 7, the hole-injection/transport layers (104B, 104G, and 104R) in the EL layers (103B, 103G, and 103R) of the light-emitting devices (550B, 550G, and 550R) are smaller than the other functional layers in the EL layers (103B, 103G, and 103R) and are covered with the functional layers stacked over the hole-injection/transport layers.

In this structure, the hole-injection/transport layers (104B, 104G, and 104R) in the EL layers are completely separated from each other by being covered with the other functional layers; thus, the insulating layers (107B, 107G, and 107R in FIG. 2A) for preventing a short circuit between the hole-injection/transport layers and the electrode 552, which are described in Structure example 1, are unnecessary.

The EL layers in this structure (the EL layers 103B, 103G, and 103R) are processed to be separated by patterning using a photolithography method; hence, end portions (side surfaces) of the processed EL layers have substantially one surface (or are positioned on substantially the same plane).

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as shown in this structure example can suppress occurrence of crosstalk between adjacent light-emitting devices.

### <Structure Example 3 of Light-Emitting Apparatus 700>

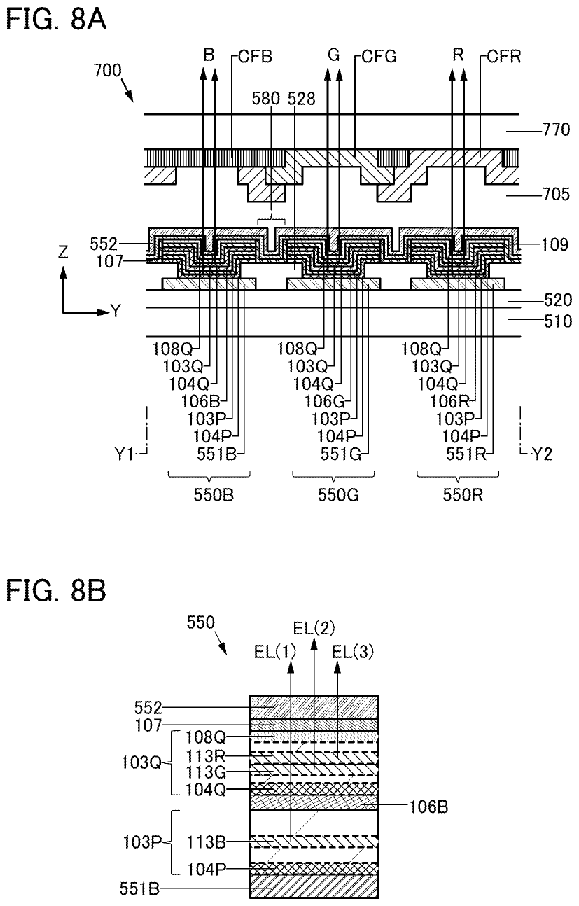

The light-emitting apparatus 700 illustrated in FIG. 8A includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, the driver circuit GD, the driver circuit SD, and the like that are composed of a plurality of transistors, and wirings that electrically connect these circuits. Note that these driver circuits are electrically connected to the light-emitting device 550B, the light-emitting device 550G, and the light-emitting device 550R, for example, to drive them. The driver circuit GD and the driver circuit SD will be described in Embodiment 4.

The light-emitting device 550B, the light-emitting device 550G, and the light-emitting device 550R each have the device structure described in Embodiment 2. Specifically, each of the light-emitting devices includes the stacked EL layers 103 to have the structure illustrated in FIG. 1B, i.e., a tandem structure.

The light-emitting device 550B has a stacked-layer structure illustrated in FIG. 8A, which includes the electrode 551B, the electrode 552, EL layers (103P and 103Q), a charge-generation layer 106B, and the insulating layer 107. Note that a specific structure of each layer is as described in Embodiment 2. The electrode 551B and the electrode 552 overlap each other. The EL layer 103P and the EL layer 103Q are stacked with the charge-generation layer 106B therebetween, and the EL layer 103P, the EL layer 103Q, and the charge-generation layer 106B are positioned between the electrode 551B and the electrode 552. Note that each of the EL layers 103P and 103Q has a stacked-layer structure of layers having different functions, including a light-emitting layer, like the EL layers 103, 103a, 103b, and 103c described in Embodiment 2. The EL layer 103P is capable of emitting blue light, for example, and the EL layer 103Q is capable of emitting yellow light, for example.

FIG. 8A illustrates only a hole-injection/transport layer 104P as a layer included in the EL layer 103P and only a hole-injection/transport layer 104Q, an electron-transport layer 108Q, and the electron-injection layer 109 as layers included in the EL layer 103Q. Thus, in the following description, the term “EL layer” (the EL layer 103P and the EL layer 103Q) is used for convenience to describe the layers included in the EL layer as well. The electron-transport layer 108Q may have a stacked-layer structure, and may include a hole-blocking layer for blocking holes that move from the anode side to the cathode side through the light-emitting layer. The electron-injection layer 109 may have a stacked-layer structure in which some or all of layers are formed using different materials.

The insulating layer 107 is formed while a sacrifice layer formed over some layers of the EL layer 103Q (in this embodiment, the layers up to the electron-transport layer 108Q over the light-emitting layer) remains over the electrode 551B as illustrated in FIG. 8A. Thus, the insulating layer 107 is formed in contact with side surfaces (or end portions) of the above layers in the EL layer 103Q, the EL layer 103P, and the charge-generation layer 106B. Accordingly, it is possible to inhibit entry of oxygen, moisture, or constituent elements thereof into the inside through the side surfaces of the EL layer 103P, the EL layer 103Q, and the charge-generation layer 106B. For the insulating layer 107, aluminum oxide, magnesium oxide, hafnium oxide, gallium oxide, indium gallium zinc oxide, silicon nitride, or silicon nitride oxide can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103Q (the layers up to the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551B and the electrode 552 have an overlap region. The EL layer 103P, the EL layer 103Q, and the charge-generation layer 106B are positioned between the electrode 551B and the electrode 552. Thus, the electron-injection layer 109 is positioned at the side surfaces (or end portions) of the EL layer 103Q, the EL layer 103P, and the charge-generation layer 106B with the insulating layer 107 therebetween, or the electrode 552 is positioned at the side surfaces (or end portions) of the EL layer 103Q, the EL layer 103P, and the charge-generation layer 106B with the electron-injection layer 109 and the insulating layer 107 therebetween. Consequently, the EL layer 103P and the electrode 552, specifically the hole-injection/transport layer 104P in the EL layer 103P and the electrode 552 can be prevented from being electrically short-circuited. In addition, the EL layer 103Q and the electrode 552, specifically the hole-injection/transport layer 104Q in the EL layer 103Q and the electrode 552 can be prevented from being electrically short-circuited. Moreover, the charge-generation layer 106B and the electrode 552 can be prevented from being electrically short-circuited.

The light-emitting device 550G has a stacked-layer structure illustrated in FIG. 8A, which includes the electrode 551G, the electrode 552, the EL layers (103P and 103Q), a charge-generation layer 106G, and the insulating layer 107. Note that a specific structure of each layer is as described in Embodiment 2. The electrode 551G and the electrode 552 overlap each other. The EL layer 103P and the EL layer 103Q are stacked with the charge-generation layer 106G therebetween, and the EL layer 103P, the EL layer 103Q, and the charge-generation layer 106G are positioned between the electrode 551G and the electrode 552.

The insulating layer 107 is formed while a sacrifice layer formed over some layers of the EL layer 103Q (in this embodiment, the layers up to the electron-transport layer 108Q over the light-emitting layer) remains over the electrode 551G as illustrated in FIG. 8A. Thus, the insulating layer 107 is formed in contact with side surfaces (or end portions) of the above layers in the EL layer 103Q, the EL layer 103P, and the charge-generation layer 106G. Accordingly, it is possible to inhibit entry of oxygen, moisture, or constituent elements thereof into the inside through the side surfaces of the EL layer 103P, the EL layer 103Q, and the charge-generation layer 106G. For the insulating layer 107, aluminum oxide, magnesium oxide, hafnium oxide, gallium oxide, indium gallium zinc oxide, silicon nitride, or silicon nitride oxide can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103Q (the layers up to the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551G and the electrode 552 have an overlap region. The EL layer 103P, the EL layer 103Q, and the charge-generation layer 106G are positioned between the electrode 551G and the electrode 552. Thus, the electron-injection layer 109 is positioned at the side surfaces (or end portions) of the EL layer 103Q, the EL layer 103P, and the charge-generation layer 106G with the insulating layer 107 therebetween, or the electrode 552 is positioned at the side surfaces (or end portions) of the EL layer 103Q, the EL layer 103P, and the charge-generation layer 106G with the electron-injection layer 109 and the insulating layer 107 therebetween. Consequently, the EL layer 103P and the electrode 552, specifically the hole-injection/transport layer 104P in the EL layer 103P and the electrode 552 can be prevented from being electrically short-circuited. In addition, the EL layer 103Q and the electrode 552, specifically the hole-injection/transport layer 104Q in the EL layer 103Q and the electrode 552 can be prevented from being electrically short-circuited. Moreover, the charge-generation layer 106G and the electrode 552 can be prevented from being electrically short-circuited.

The light-emitting device 550R has a stacked-layer structure illustrated in FIG. 8A, which includes the electrode 551R, the electrode 552, the EL layers (103P and 103Q), a charge-generation layer 106R, and the insulating layer 107. Note that a specific structure of each layer is as described in Embodiment 2. The electrode 551R and the electrode 552 overlap each other. The EL layer 103P and the EL layer 103Q are stacked with the charge-generation layer 106R therebetween, and the EL layer 103P, the EL layer 103Q, and the charge-generation layer 106R are positioned between the electrode 551R and the electrode 552.

The insulating layer 107 is formed while a sacrifice layer formed over some layers of the EL layer 103Q (in this embodiment, the layers up to the electron-transport layer 108Q over the light-emitting layer) remains over the electrode 551R as illustrated in FIG. 8A. Thus, the insulating layer 107 is formed in contact with side surfaces (or end portions) of the above layers in the EL layer 103Q, the EL layer 103P, and the charge-generation layer 106R. Accordingly, it is possible to inhibit entry of oxygen, moisture, or constituent elements thereof into the inside through the side surfaces of the EL layer 103P, the EL layer 103Q, and the charge-generation layer 106R. For the insulating layer 107, aluminum oxide, magnesium oxide, hafnium oxide, gallium oxide, indium gallium zinc oxide, silicon nitride, or silicon nitride oxide can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover some layers of the EL layer 103Q (the layers up to the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551R and the electrode 552 have an overlap region. The EL layer 103P, the EL layer 103Q, and the charge-generation layer 106R are positioned between the electrode 551R and the electrode 552. Thus, the electron-injection layer 109 is positioned at the side surfaces (or end portions) of the EL layer 103Q, the EL layer 103P, and the charge-generation layer 106R with the insulating layer 107 therebetween, or the electrode 552 is positioned at the side surfaces (or end portions) of the EL layer 103Q, the EL layer 103P, and the charge-generation layer 106R with the electron-injection layer 109 and the insulating layer 107 therebetween. Consequently, the EL layer 103P and the electrode 552, specifically the hole-injection/transport layer 104P in the EL layer 103P and the electrode 552 can be prevented from being electrically short-circuited. In addition, the EL layer 103Q and the electrode 552, specifically the hole-injection/transport layer 104Q in the EL layer 103Q and the electrode 552 can be prevented from being electrically short-circuited. Moreover, the charge-generation layer 106R and the electrode 552 can be prevented from being electrically short-circuited.

The EL layers (103P and 103Q) and the charge-generation layers (106B, 106G, and 106R) included in the light-emitting devices are processed to be separated between the light-emitting devices by patterning using a photolithography method; thus, the end portions (side surfaces) of the processed EL layers have substantially one surface (or are positioned on substantially the same plane).

The space 580 is provided between the EL layers (103P and 103Q) and the charge-generation layer (106B, 106G, or 106R) in one light-emitting device and those in the adjacent light-emitting device. The charge-generation layers (106B, 106G, and 106R) and the hole-injection layers included in the hole-transport regions in the EL layers (103P and 103Q) often have high conductivity; therefore, these layers formed as layers shared by adjacent light-emitting devices might cause crosstalk. Thus, providing the space 580 as shown in this structure example can suppress occurrence of crosstalk between adjacent light-emitting devices.

When electrical continuity is established between the EL layer 103B, the EL layer 103G, and the EL layer 103R in a light-emitting apparatus (display panel) with a high resolution exceeding 1000 ppi, crosstalk occurs, resulting in a narrower color gamut that the light-emitting apparatus is capable of reproducing. Providing the space 580 in a high-resolution display panel with more than 1000 ppi, preferably more than 2000 ppi, or further preferably in an ultrahigh-resolution display panel with more than 5000 ppi allows the display panel to express vivid colors.

In this structure example, the light-emitting device 550B, the light-emitting device 550G, and the light-emitting device 550R each emit white light. In this specification and the like, a light-emitting device capable of emitting white light is called a white-light-emitting device in some cases. Note that a combination of such a white-light-emitting device with coloring layers (e.g., color filters) enables providing a full-color display apparatus. Accordingly, the second substrate 770 includes a coloring layer CFB, a coloring layer CFG, and a coloring layer CFR. Note that these coloring layers may be provided to partly overlap each other as illustrated in FIG. 8A. When the coloring layers partly overlap each other, the overlap portion can function as a light-blocking film. In this structure example, a material that preferentially transmits blue light (B) is used for the coloring layer CFB, a material that preferentially transmits green light (G) is used for the coloring layer CFG, and a material that preferentially transmits red light (R) is used for the coloring layer CFR, for example.

FIG. 8B illustrates a structure of the light-emitting device 550B in the case where each of the light-emitting devices 550B, 550G, and 550R (collectively referred to as light-emitting devices 550) is a white-light-emitting device. The EL layer 103P and the EL layer 103Q are stacked over the electrode 551B, with the charge-generation layer 106B between the EL layers. The EL layer 103P includes the light-emitting layer 113B that emits blue light EL(1), and the EL layer 103Q includes the light-emitting layer 113G that emits green light EL(2) and the light-emitting layer 113R that emits red light EL(3).

Note that a color conversion layer can be used instead of the coloring layer. For example, nanoparticles or quantum dots can be used for the color conversion layer.

For example, a color conversion layer that converts blue light into green light can be used instead of the coloring layer CFG. Thus, blue light emitted from the light-emitting device 550G can be converted into green light. Moreover, a color conversion layer that converts blue light into red light can be used instead of the coloring layer CFR. Thus, blue light emitted from the light-emitting device 550R can be converted into red light.

When the above-described light-emitting device having an SBS structure and the white-light-emitting device are compared to each other, the former can have lower power consumption than the latter. To reduce power consumption, a light-emitting device having an SBS structure is preferably used. Meanwhile, the white-light-emitting device is preferable in terms of lower manufacturing cost or higher manufacturing yield because the manufacturing process of the white-light-emitting device is simpler than that of a light-emitting device having an SBS structure.

### <Structure Example 4 of Light-Emitting Apparatus 700>

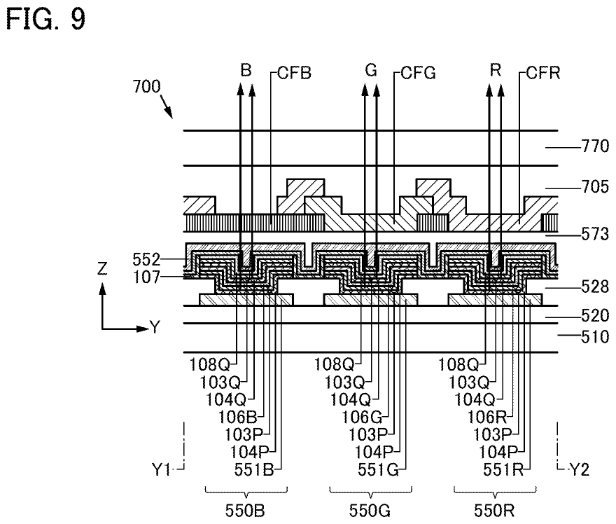

The light-emitting apparatus (display panel) 700 illustrated in FIG. 9 includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, the driver circuit GD, the driver circuit SD, and the like that are composed of a plurality of transistors, and wirings that electrically connect these circuits. Note that these driver circuits are electrically connected to the light-emitting device 550B, the light-emitting device 550G, and the light-emitting device 550R, for example, to drive them. The driver circuit GD and the driver circuit SD will be described in Embodiment 4.

The light-emitting device 550B, the light-emitting device 550G, and the light-emitting device 550R each have the device structure described in Embodiment 2. This is suitable particularly for the case where each of the light-emitting devices includes the stacked EL layers 103 to have the structure illustrated in FIG. 1B, i.e., a tandem structure.

Note that specific structures of the light-emitting devices illustrated in FIG. 9 are the same as the structures of the light-emitting devices 550B, 550G, and 550R described with reference to FIG. 8A, and each of the light-emitting devices emits white light.

The light-emitting apparatus in this structure example is different from the light-emitting apparatus illustrated in FIG. 8A in including the coloring layer CFB, the coloring layer CFG, and the coloring layer CFR formed over the light-emitting devices over the first substrate 510.

In other words, an insulating layer 573 is provided over the electrode 552 of each light-emitting device formed over the first substrate 510, and the coloring layer CFB, the coloring layer CFG, and the coloring layer CFR are provided over the insulating layer 573.

The insulating layer 705 is provided over the coloring layer CFB, the coloring layer CFG, and the coloring layer CFR. The insulating layer 705 includes a region sandwiched between the second substrate 770 and the first substrate 510 on the side closer to the coloring layers (CFB, CFG, and CFR), which is provided with the functional layer 520, the light-emitting devices (550B, 550G, and 550R), and the coloring layers CFB, CFG, and CFR. The insulating layer 705 has a function of attaching the first substrate 510 and the second substrate 770.

For the insulating layer 573 and the insulating layer 705, an inorganic material, an organic material, a composite material of an inorganic material and an organic material, or the like can be used.

As the inorganic material, an inorganic oxide film, an inorganic nitride film, an inorganic oxynitride film, and the like, or a layered material obtained by stacking some of these films can be used. For example, a film including any of a silicon oxide film, a silicon nitride film, a silicon oxynitride film, an aluminum oxide film, and the like, or a film including a material obtained by stacking any of these films can be used. Note that a silicon nitride film is a dense film and has an excellent function of inhibiting diffusion of impurities. Alternatively, for an oxide semiconductor (e.g., an IGZO film), a stacked-layer structure of an aluminum oxide film and an IGZO film over the aluminum oxide film, for example, can be used.

As the organic material, polyester, polyolefin, polyamide, polyimide, polycarbonate, polysiloxane, acrylic, and the like, or a layered material or a composite material including two or more of resins selected from the above can be used. Alternatively, an organic material such as a reactive curable adhesive, a photo-curable adhesive, a thermosetting adhesive, and/or an anaerobic adhesive can be used.

### <Example 2 of Method for Manufacturing Light-Emitting Apparatus>

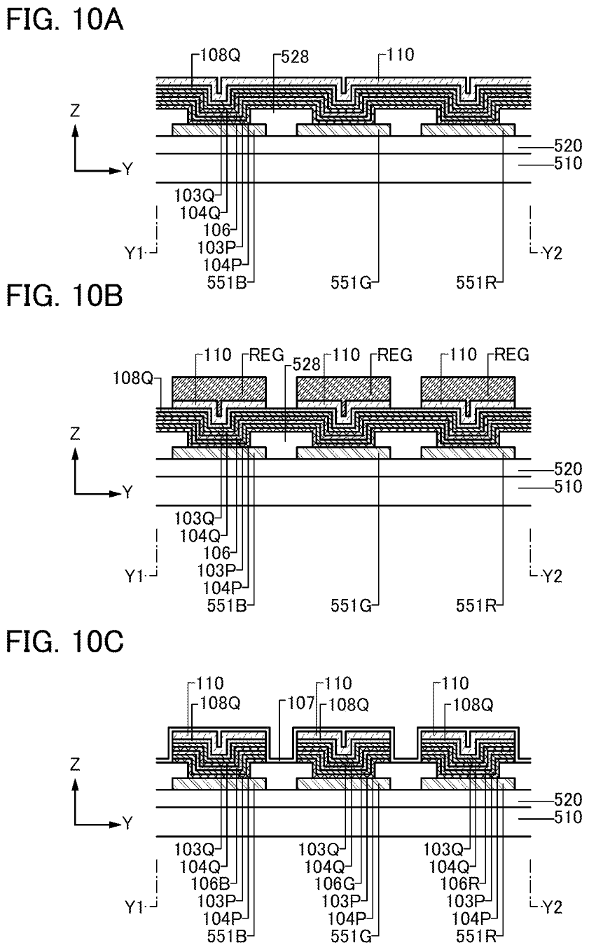

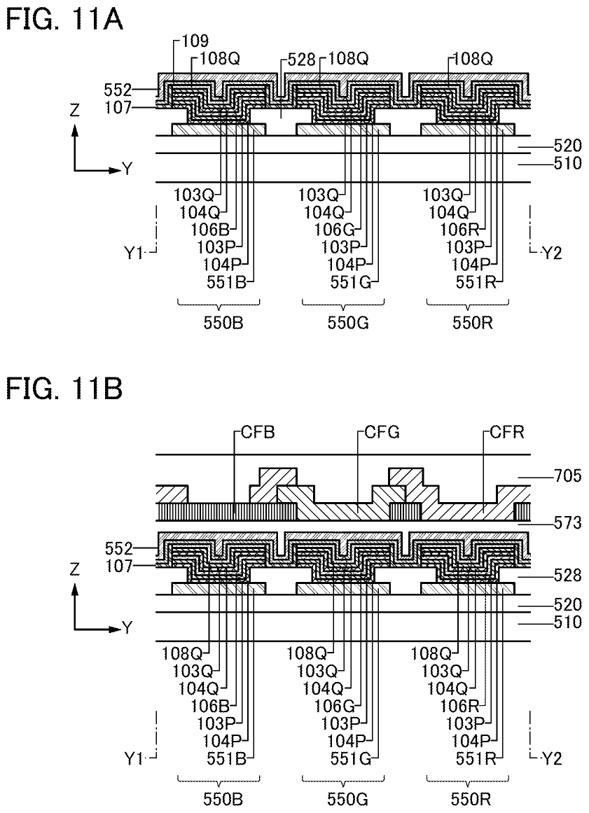

Next, a method for manufacturing the light-emitting apparatus illustrated in FIG. 9 will be described with reference to FIGS. 10A to 10C and FIGS. 11A and 11B.

As illustrated in FIG. 10A, over the electrodes (551B, 551G, and 551R) and the partition 528 (see FIG. 3B) formed over the first substrate 510, the EL layer 103P (including the hole-injection/transport layer 104P), the charge-generation layer 106, and the EL layer 103Q (including the hole-injection/transport layer 104Q and the electron-transport layer 108Q) are formed so as to cover the electrodes and the partition 528. Furthermore, the sacrifice layer 110 is formed over the EL layer 103Q. Description of the structure of the sacrifice layer 110 is not made because the structure is similar to that described with reference to FIG. 4A.

Then, as illustrated in FIG. 10B, the resist masks REG are formed in the following manner: a resist is applied onto the sacrifice layer 110, and the resist in the regions of the sacrifice layer 110 which do not overlap the electrode 551B, the electrode 551G, or the electrode 551R is removed, whereby the resist remains in the regions of the sacrifice layer 110 which overlap the electrode 551B, the electrode 551G, and the electrode 551R. For example, the resist applied onto the sacrifice layer 110 is formed into desired shapes by a photolithography method. Then, portions of the sacrifice layer 110 not covered with the thus formed resist masks REG are removed by etching. After that, the resist masks REG are removed, and portions of the EL layer 103P (including the hole-injection/transport layer 104P), portions of the charge-generation layer 106, and portions of the EL layer 103Q (including the hole-injection/transport layer 104Q and the electron-transport layer 108Q) which are not covered with the sacrifice layers are removed by etching, whereby the EL layer 103P, the charge-generation layer 106, and the EL layer 103Q are processed to have side surfaces (or have their side surfaces exposed) or have a belt-like shape that extends in the direction intersecting the sheet of the diagram. Specifically, dry etching is performed with the use of the sacrifice layers 110 formed in patterns over the EL layer 103Q (including the hole-injection/transport layer 104Q and the electron-transport layer 108Q) (see FIG. 10C). Although not shown in FIG. 10C, in the case where the sacrifice layers 110 each have the stacked-layer structure of the first sacrifice layer and the second sacrifice layer, the EL layer 103Q (including the hole-injection/transport layer 104Q and the electron-transport layer 108Q), the charge-generation layer 106, and the EL layer 103P (including the hole-injection/transport layer 104P) may be processed into a predetermined shape in the following manner as in the description with reference to FIG. 4A: part of the second sacrifice layer is etched with the use of the resist mask REG, the resist mask REG is then removed, and part of the first sacrifice layer is etched with the use of the second sacrifice layer as a mask. The partition 528 can be used as an etching stopper.

Then, the insulating layer 107 is formed over the sacrifice layers 110, the EL layers (103P and 103Q), and the partition 528. For example, the insulating layer 107 is formed by an ALD method over the sacrifice layers 110, the EL layers (103P and 103Q), and the partition 528 so as to cover them. In this case, the insulating layer 107 is formed in contact with the side surfaces of the EL layers (103P and 103Q) as illustrated in FIG. 10C. Specifically, the insulating layer 107 is formed on side surfaces that are exposed when the EL layer 103P (including the hole-injection/transport layer 104P), the charge-generation layer 106, and the EL layer 103Q (including the hole-injection/transport layer 104Q and the electron-transport layer 108Q) are processed by etching. This can inhibit entry of oxygen, moisture, or constituent elements thereof into the inside through the side surfaces of the EL layers (103P and 103Q). Examples of the material used for the insulating layer 107 include aluminum oxide, magnesium oxide, hafnium oxide, gallium oxide, indium gallium zinc oxide, silicon nitride, and silicon nitride oxide. For the insulating layer 107, the hole-transport material described in Embodiment 2 can be used.

Then, as illustrated in FIG. 11A, the sacrifice layers 110 are removed, and the electron-injection layer 109 is formed over the insulating layers 107 and the electron-transport layers 108Q. The electron-injection layer 109 is formed by a vacuum evaporation method, for example. The electron-injection layer 109 is positioned at the side surfaces of some layers of the EL layers (103P and 103Q) (including the hole-injection/transport layers (104P and 104Q), the light-emitting layers, and the electron-transport layers (108P and 108Q)) and the charge-generation layers (106B, 106G, and 106R) with the insulating layers 107 therebetween.

Next, the electrode 552 is formed over the electron-injection layer 109. The electrode 552 is formed by a vacuum evaporation method, for example. The electrode 552 is positioned at the side surfaces (or end portions) of some layers of the EL layers (103P and 103Q) (including the hole-injection/transport layers (104P and 104Q), the light-emitting layers, and the electron-transport layers (108P and 108Q)) and the charge-generation layers (106B, 106G, and 106R) with the electron-injection layer 109 and the insulating layers 107 therebetween. Thus, the EL layers (103P and 103Q) and the electrode 552, specifically the hole-injection/transport layers (104P and 104Q) in the EL layers (103P and 103Q) and the electrode 552 can be prevented from being electrically short-circuited.

In the above manner, the EL layers 103P (each including the hole-injection/transport layer 104P), the charge-generation layers (106B, 106G, and 106R), and the EL layers 103Q (each including the hole-injection/transport layer 104Q and the electron-transport layer 108Q) of the light-emitting device 550B, the light-emitting device 550G, and the light-emitting device 550R can be separately formed by one patterning using a photolithography method.

Next, the insulating layer 573, the coloring layer CFB, the coloring layer CFG, the coloring layer CFR, and the insulating layer 705 are formed (see FIG. 11B).

For example, the insulating layer 573 is formed by stacking a flat film and a dense film. Specifically, the flat film is formed by a coating method, and the dense film is formed over the flat film by a chemical vapor deposition method, an atomic layer deposition (ALD) method, or the like. Thus, the insulating layer 573 with few defects and high quality can be formed.

The coloring layer CFB, the coloring layer CFG, and the coloring layer CFR are formed to have predetermined shapes by using a color resist, for example. Note that the coloring layers are processed so that the coloring layer CFR and the coloring layer CFB overlap each other over the partition 528. This can suppress a phenomenon in which light emitted from one light-emitting device enters an adjacent light-emitting device.

For the insulating layer 705, an inorganic material, an organic material, a composite material of an inorganic material and an organic material, or the like can be used.

The EL layers (103P and 103Q) and the charge-generation layers (106B, 106G, and 106R) included in the light-emitting devices are processed to be separated between the light-emitting devices by patterning using a photolithography method; thus, a high-resolution light-emitting apparatus (display panel) can be fabricated. The end portions (side surfaces) of the EL layers processed by patterning using a photolithography method have substantially one surface (or are positioned on substantially the same plane).

The charge-generation layers (106B, 106G, and 106R) and the hole-injection layers included in the hole-transport regions in the EL layers (103P and 103Q) often have high conductivity; therefore, these layers formed as layers shared by adjacent light-emitting devices might cause crosstalk. Thus, processing the EL layers to be separated by patterning using a photolithography method as shown in this structure example can suppress occurrence of crosstalk between adjacent light-emitting devices.

### <Structure Example 5 of Light-Emitting Apparatus 700>

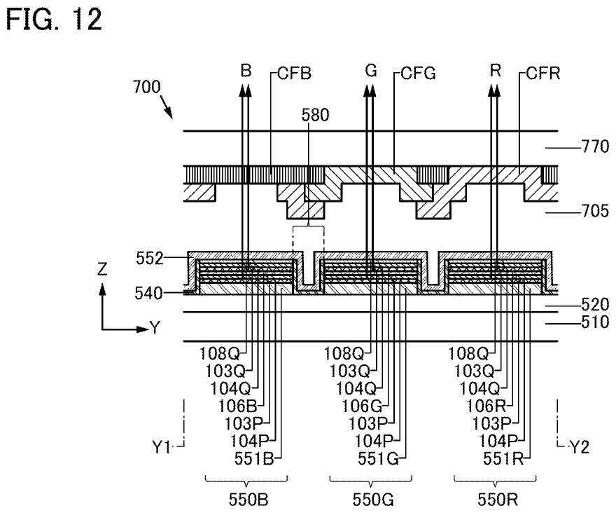

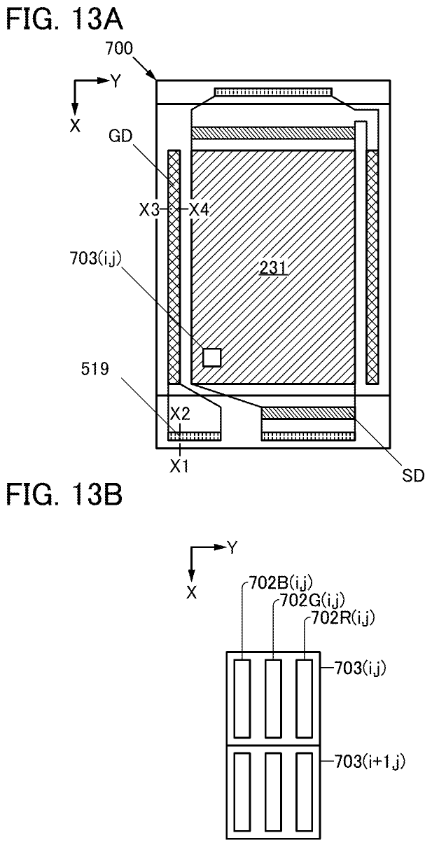

The light-emitting apparatus (display panel) 700 illustrated in FIG. 12 includes the light-emitting device 550B, the light-emitting device 550G, and the light-emitting device 550R. The light-emitting device 550B, the light-emitting device 550G, and the light-emitting device 550R are formed over the functional layer 520 provided over the first substrate 510. The functional layer 520 includes, for example, the driver circuit GD, the driver circuit SD, and the like that are composed of a plurality of transistors, and wirings that electrically connect these circuits. Note that these driver circuits are electrically connected to the light-emitting device 550B, the light-emitting device 550G, and the light-emitting device 550R, for example, to drive them. The driver circuit GD and the driver circuit SD will be described in Embodiment 4.

The light-emitting device 550B, the light-emitting device 550G, and the light-emitting device 550R each have the device structure described in Embodiment 2. This is suitable particularly for the case where each of the light-emitting devices includes the stacked EL layers 103 to have the structure illustrated in FIG. 1B, i.e., a tandem structure.

As illustrated in FIG. 12, the space 580 is provided between the light-emitting devices, for example, between the light-emitting device 550B and the light-emitting device 550G. An insulating layer 540 is formed in the space 580.

For example, the insulating layer 540 can be formed in the space 580 by a photolithography method after the EL layers 103P (each including the hole-injection/transport layer 104P), the charge-generation layers (106B, 106G, and 106R), and the EL layers 103Q (each including the hole-injection/transport layer 104Q and the electron-transport layer 108Q) are separately formed by patterning using a photolithography method. Furthermore, the electrode 552 can be formed over the EL layers 103Q (each including the hole-injection/transport layer 104Q and the electron-transport layer 108Q) and the insulating layer 540.

In this structure, the EL layers are separated from each other by the insulating layer 540; thus, the insulating layer described in Structure example 3 (the insulating layer 107 in FIGS. 8A and 8B) is unnecessary.

The EL layers (103P and 103Q) and the charge-generation layers (106B, 106G, and 106R) included in the light-emitting devices are processed to be separated between the light-emitting devices by patterning using a photolithography method; thus, a high-resolution light-emitting apparatus (display panel) can be fabricated. The end portions (side surfaces) of the EL layers processed by patterning using a photolithography method have substantially one surface (or are positioned on substantially the same plane).

The charge-generation layers (106B, 106G, and 106R) and the hole-injection layers included in the hole-transport regions in the EL layers (103P and 103Q) often have high conductivity; therefore, these layers formed as layers shared by adjacent light-emitting devices might cause crosstalk. Thus, processing the EL layers to be separated by patterning using a photolithography method as shown in this structure example can suppress occurrence of crosstalk between adjacent light-emitting devices.

In this structure example, the adjacent light-emitting devices (the light-emitting device 550B, the light-emitting device 550G, and the light-emitting device 550R) may be fabricated by the fabrication method described with reference to FIGS. 3A and 3B to FIGS. 6A and 6B. In that case, the EL layers (103P and 103Q) and the charge-generation layers (106R, 106G, and 106R) of the light-emitting devices can be separately formed, which allows the EL layers (103P and 103Q) having different structures to be formed. For example, the EL layers (103P and 103Q) of the light-emitting device 550B may be formed as blue-light-emitting layers by including a blue-light-emitting substance, the EL layers (103P and 103Q) of the light-emitting device 550G may be formed as green-light-emitting layers by including a green-light-emitting substance, and the EL layers (103P and 103Q) of the light-emitting device 550R may be formed as red-light-emitting layers by including a red-light-emitting substance. Alternatively, the EL layer (103P) and the EL layer (103Q) of each of the light-emitting device 550B, the light-emitting device 550G, and the light-emitting device 550R may be formed using light-emitting substances exhibiting different emission colors.

The structures described in this embodiment can be used in appropriate combination with any of the structures described in the other embodiments.

### Embodiment 4

In this embodiment, a light-emitting apparatus of one embodiment of the present invention will be described with reference to FIGS. 13A and 13B, FIGS. 14A and 14B, and FIGS. 15A and 15B. The light-emitting apparatus 700 illustrated in FIGS. 13A and 13B, FIGS. 14A and 14B, and FIGS. 15A and 15B includes the light-emitting device described in Embodiment 2. The light-emitting apparatus 700 described in this embodiment can be referred to as a display panel because it can be used in a display unit of an electronic appliance and the like.

As illustrated in FIG. 13A, the light-emitting apparatus 700 described in this embodiment includes a display region 231, and the display region 231 includes a pixel set 703(i,j). A pixel set 703(i+1,j) adjacent to the pixel set 703(i,j) is provided as illustrated in FIG. 13B.

Note that a plurality of pixels can be used in the pixel 703(i,j). For example, a plurality of pixels that show colors of different hues can be used. Note that a plurality of pixels can be referred to as subpixels. In addition, a set of subpixels can be referred to as a pixel.

Such a structure enables additive mixture or subtractive mixture of colors shown by the plurality of pixels. Alternatively, it is possible to express a color of a hue that a single pixel cannot show.

Specifically, a pixel 702B(i,j) for showing blue, the pixel 702G(i,j) for showing green, and a pixel 702R(i,j) for showing red can be used in the pixel 703(i,j). The pixel 702B(i,j), the pixel 702G(i,j), and the pixel 702R(i,j) can each be referred to as a subpixel.

A pixel for showing white or the like in addition to the above set may be used in the pixel 703(i,j). Moreover, a pixel for showing cyan, a pixel for showing magenta, and a pixel for showing yellow may be used as subpixels in the pixel 703(i,j).

A pixel that emits infrared light in addition to the above set may be used in the pixel 703(i,j). Specifically, a pixel that emits light including light with a wavelength of greater than or equal to 650 nm and less than or equal to 1000 nm can be used in the pixel 703(i,j).

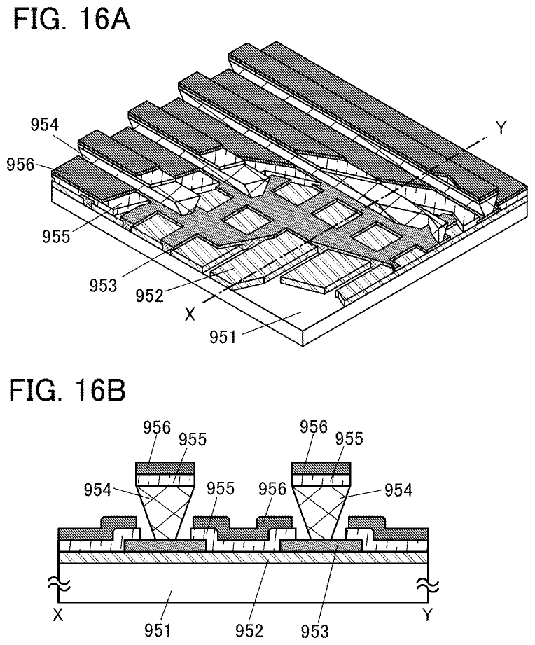

The light-emitting apparatus 700 includes the driver circuit GD and the driver circuit SD around the display region 231 in FIG. 13A. The light-emitting apparatus 700 also includes a terminal 519 electrically connected to the driver circuit GD, the driver circuit SD, and the like. The terminal 519 can be electrically connected to a flexible printed circuit FPC1 (see FIGS. 16A and 16B), for example.

The driver circuit GD has a function of supplying a first selection signal and a second selection signal. For example, the driver circuit GD is electrically connected to an after-mentioned conductive film G1(i) to supply the first selection signal, and is electrically connected to an after-mentioned conductive film G2(i) to supply the second selection signal. The driver circuit SD has a function of supplying an image signal and a control signal, and the control signal includes a first level and a second level. For example, the driver circuit SD is electrically connected to an after-mentioned conductive film S1g(j) to supply the image signal, and is electrically connected to an after-mentioned conductive film S2g(j) to supply the control signal.

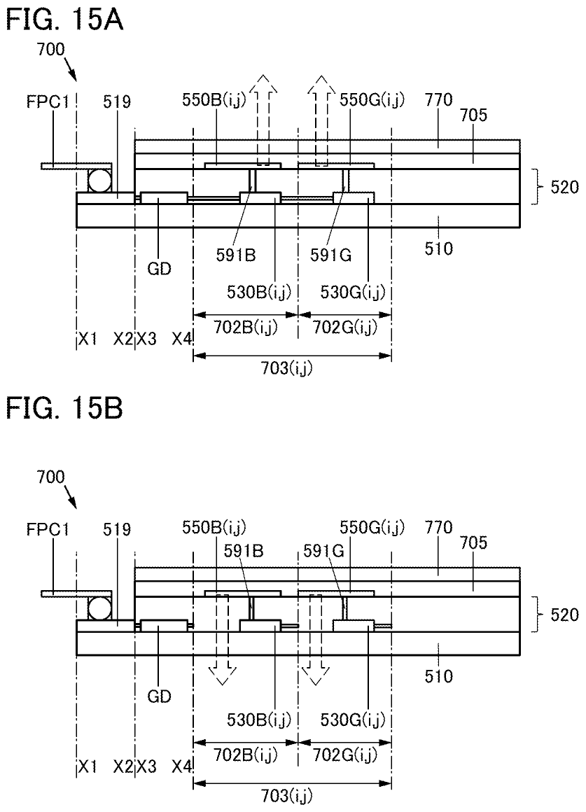

FIG. 15A shows a cross-sectional view of the light-emitting apparatus taken along each of the dashed-dotted line X1-X2 and the dashed-dotted line X3-X4 in FIG. 13A. As illustrated in FIG. 15A, the light-emitting apparatus 700 includes the functional layer 520 between the first substrate 510 and the second substrate 770. The functional layer 520 includes, for example, the driver circuit GD, the driver circuit SD, and the like that are described above and wirings that electrically connect these circuits. Although FIG. 15A illustrates the functional layer 520 including a pixel circuit 530B(i,j), a pixel circuit 530G(i,j), and the driver circuit GD, the functional layer 520 is not limited thereto.

Each pixel circuit (e.g., the pixel circuit 530B(i,j) and the pixel circuit 530G(i,j) in FIG. 15A) included in the functional layer 520 is electrically connected to a light-emitting device (e.g., a light-emitting device 550B(i,j) and a light-emitting device 550G(i,j) in FIG. 15A) formed over the functional layer 520. Specifically, the light-emitting device 550B(i,j) is electrically connected to the pixel circuit 530B(i,j) through an opening 591B, and the light-emitting device 550G(i,j) is electrically connected to the pixel circuit 530G(i,j) through an opening 591G. The insulating layer 705 is provided over the functional layer 520 and the light-emitting devices, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting apparatus of one embodiment of the present invention can be used as a touch panel.

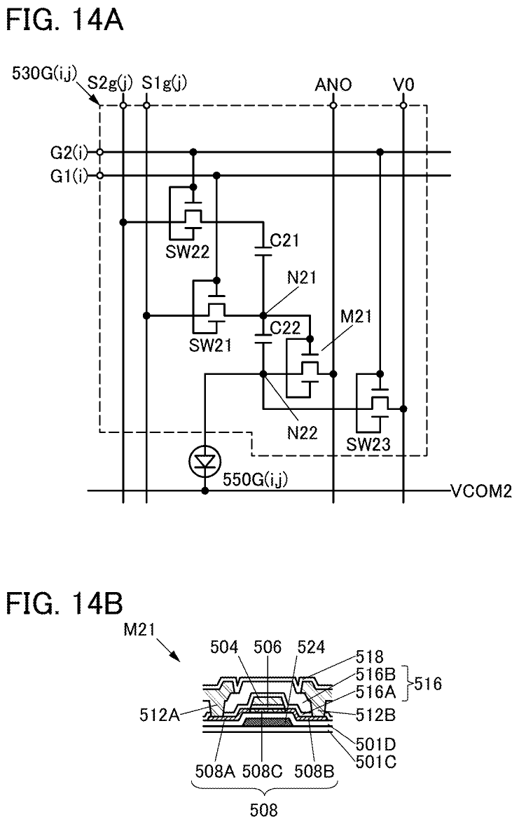

FIG. 14A illustrates a specific configuration of the pixel circuit 530G(i,j).

As illustrated in FIG. 14A, the pixel circuit 530G(i,j) includes a switch SW21, a switch SW22, a transistor M21, a capacitor C21, and a node N21. The pixel circuit 530G(i,j) also includes a node N22, a capacitor C22, and a switch SW23.

The transistor M21 includes a gate electrode electrically connected to the node N21, a first electrode electrically connected to the light-emitting device 550G(i,j), and a second electrode electrically connected to a conductive film ANO.

The switch SW21 includes a first terminal electrically connected to the node N21 and a second terminal electrically connected to the conductive film S1g(j). The switch SW21 has a function of controlling its on/off state on the basis of the potential of the conductive film G1(i).

The switch SW22 includes a first terminal electrically connected to the conductive film S2g(j), and has a function of controlling its on/off state on the basis of the potential of the conductive film G2(i).

The capacitor C21 includes a conductive film electrically connected to the node N21 and a conductive film electrically connected to a second electrode of the switch SW22.

Accordingly, an image signal can be stored in the node N21. Alternatively, the potential of the node N21 can be changed using the switch SW22. Alternatively, the intensity of light emitted from the light-emitting device 550G(i,j) can be controlled with the potential of the node N21.

FIG. 14B illustrates an example of a specific structure of the transistor M21 described in FIG. 14A. As the transistor M21, a bottom-gate transistor, a top-gate transistor, or the like can be used as appropriate.

The transistor illustrated in FIG. 14B includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other.

A conductive film 524 can be used in the transistor. The conductive film 524 includes a region where the semiconductor film 508 is positioned between the conductive film 504 and the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

For the semiconductor film 508, a semiconductor containing an element of Group 14 can be used. Specifically, a semiconductor containing silicon can be used for the semiconductor film 508.

Hydrogenated amorphous silicon can be used for the semiconductor film 508. Microcrystalline silicon or the like can also be used for the semiconductor film 508. In such cases, it is possible to provide a light-emitting apparatus having less display unevenness than a light-emitting apparatus (or a display panel) using polysilicon for the semiconductor film 508, for example. Moreover, it is easy to increase the size of the light-emitting apparatus.

Polysilicon can be used for the semiconductor film 508. In this case, for example, the field-effect mobility of the transistor can be higher than that of a transistor using hydrogenated amorphous silicon for the semiconductor film 508. For another example, the driving capability can be higher than that of a transistor using hydrogenated amorphous silicon for the semiconductor film 508. For another example, the aperture ratio of the pixel can be higher than that in the case of employing a transistor using hydrogenated amorphous silicon for the semiconductor film 508.

For another example, the reliability of the transistor can be higher than that of a transistor using hydrogenated amorphous silicon for the semiconductor film 508.

The temperature required for fabricating the transistor can be lower than that required for a transistor using single crystal silicon, for example.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Single crystal silicon can be used for the semiconductor film 508. In this case, for example, the resolution can be higher than that of a light-emitting apparatus (or a display panel) using hydrogenated amorphous silicon for the semiconductor film 508. For another example, it is possible to provide a light-emitting apparatus having less display unevenness than a light-emitting apparatus using polysilicon for the semiconductor film 508. For another example, smart glasses or a head-mounted display can be provided.

A metal oxide can be used for the semiconductor film 508. In this case, the pixel circuit can hold an image signal for a longer time than a pixel circuit including a transistor that uses hydrogenated amorphous silicon for the semiconductor film. Specifically, a selection signal can be supplied at a frequency of lower than 30 Hz, preferably lower than 1 Hz, further preferably less than once per minute while flickering is suppressed. Consequently, fatigue of a user of an electronic appliance can be reduced. Furthermore, power consumption for driving can be reduced.

An oxide semiconductor can be used for the semiconductor film 508. Specifically, an oxide semiconductor containing indium, an oxide semiconductor containing indium, gallium, and zinc, or an oxide semiconductor containing indium, gallium, zinc, and tin can be used for the semiconductor film 508.

The use of an oxide semiconductor for the semiconductor film achieves a transistor having a lower leakage current in the off state than a transistor using hydrogenated amorphous silicon for the semiconductor film. Thus, a transistor using an oxide semiconductor for the semiconductor film is preferably used as a switch or the like. Note that a circuit in which a transistor using an oxide semiconductor for the semiconductor film is used as a switch is capable of retaining the potential of a floating node for a longer time than a circuit in which a transistor using hydrogenated amorphous silicon for the semiconductor film is used as a switch.

Although the light-emitting apparatus in FIG. 15A has a structure in which light is extracted from the second substrate 770 side (top emission structure), a light-emitting apparatus may have a structure in which light is extracted from the first substrate 510 side (bottom emission structure) as illustrated in FIG. 15B. In a bottom-emission light-emitting apparatus, the first electrode 101 is formed as a transflective electrode and the second electrode 102 is formed as a reflective electrode.

Although FIGS. 15A and 15B illustrate active-matrix light-emitting apparatuses, the structure of the light-emitting device described in Embodiment 2 may be applied to a passive-matrix light-emitting apparatus illustrated in FIGS. 16A and 16B.

FIG. 16A is a perspective view illustrating the passive-matrix light-emitting apparatus, and FIG. 16B shows a cross section along the line X-Y in FIG. 16A. In FIGS. 16A and 16B, an electrode 952 and an electrode 956 are provided over a substrate 951, and an EL layer 955 is provided between the electrode 952 and the electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section of the partition layer 954 in the short axis direction is trapezoidal, and the lower base (the side in contact with the insulating layer 953) is shorter than the upper base. The partition layer 954 thus provided can prevent defects in the light-emitting device due to static electricity or the like.

The structures described in this embodiment can be used in appropriate combination with any of the structures described in the other embodiments.

### Embodiment 5

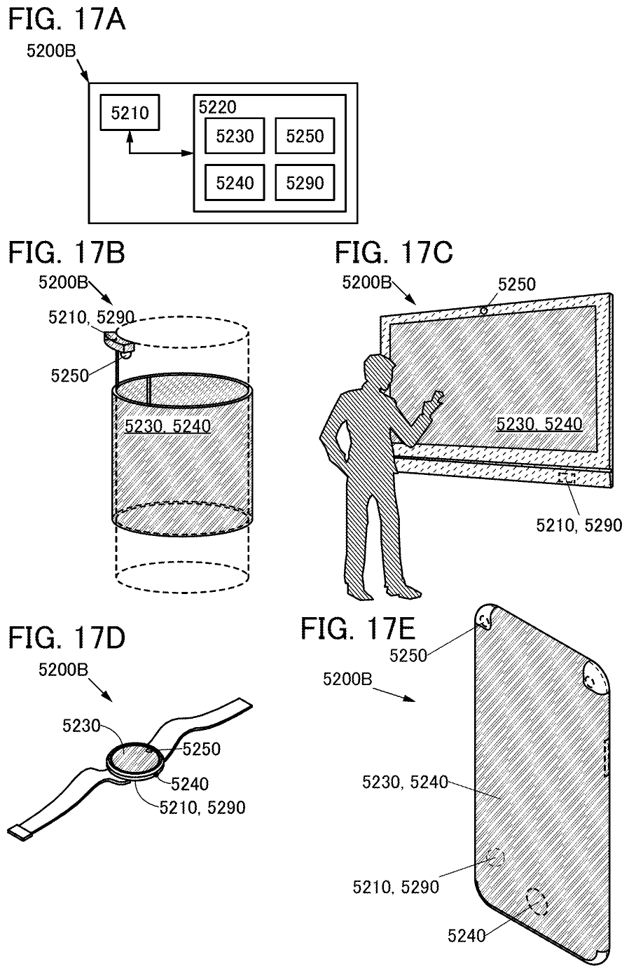

In this embodiment, structures of electronic appliances of embodiments of the present invention will be described with reference to FIGS. 17A to 17E, FIGS. 18A to 18E, and FIGS. 19A and 19B.

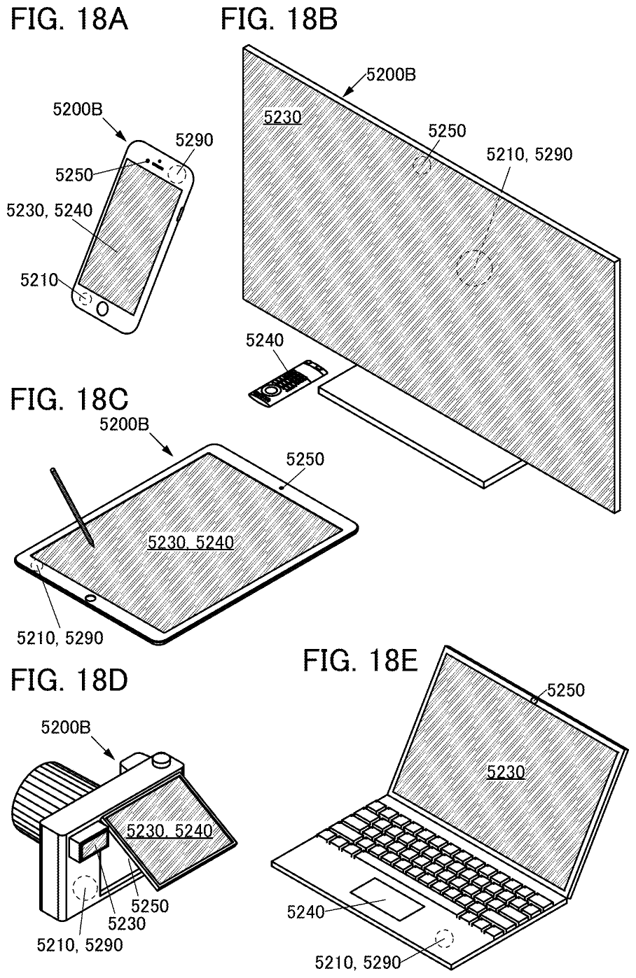

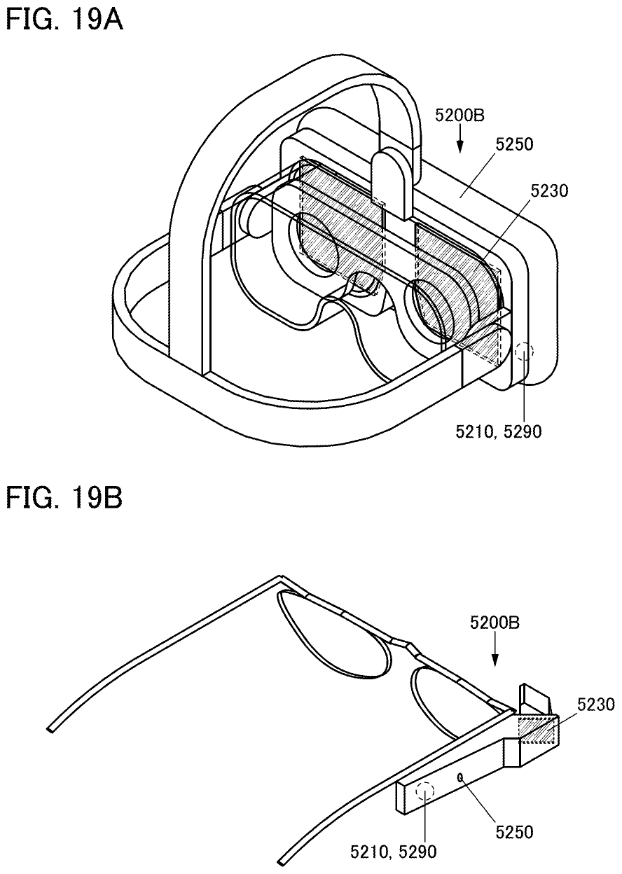

FIGS. 17A to 17E, FIGS. 18A to 18E, and FIGS. 19A and 19B each illustrate a structure of the electronic appliance of one embodiment of the present invention. FIG. 17A is a block diagram of the electronic appliance and FIGS. 17B to 17E are perspective views illustrating structures of the electronic appliance. FIGS. 18A to 18E are perspective views illustrating structures of the electronic appliance. FIGS. 19A and 19B are perspective views illustrating structures of the electronic appliance.

An electronic appliance 5200B described in this embodiment includes an arithmetic device 5210 and an input/output device 5220 (see FIG. 17A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic appliance 5200B.

Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 2 can be used for the display unit 5230.

The sensor unit 5250 has a function of supplying sensing data. For example, the sensor unit 5250 has a function of sensing a surrounding environment where the electronic appliance is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic appliance or a communication network by wireless communication or wired communication. Specifically, the communication unit 5290 has a function of wireless local area network communication, telephone communication, near field communication, or the like.

FIG. 17B illustrates an electronic appliance having an outer shape along a cylindrical column or the like. An example of such an electronic appliance is digital signage. The display panel of one embodiment of the present invention can be used for the display unit 5230. The electronic appliance may have a function of changing its display method in accordance with the illuminance of a usage environment. The electronic appliance has a function of changing the displayed content when sensing the existence of a person. Thus, for example, the electronic appliance can be provided on a column of a building. The electronic appliance can display advertising, guidance, or the like.

FIG. 17C illustrates an electronic appliance having a function of generating image data on the basis of the path of a pointer used by the user. Examples of such an electronic appliance include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 17D illustrates an electronic appliance that is capable of receiving data from another device and displaying the data on the display unit 5230. An example of such an electronic appliance is a wearable electronic appliance. Specifically, the electronic appliance can display several options, and the user can choose some from the options and send a reply to the data transmitter. As another example, the electronic appliance has a function of changing its display method in accordance with the illuminance of a usage environment. Thus, for example, power consumption of the wearable electronic appliance can be reduced. As another example, the wearable electronic appliance can display an image so as to be suitably used even in an environment under strong external light, e.g., outdoors in fine weather.

FIG. 17E illustrates an electronic appliance including the display unit 5230 having a surface gently curved along a side surface of a housing. An example of such an electronic appliance is a mobile phone. The display unit 5230 includes a display panel that has a function of displaying images on the front surface, the side surfaces, the top surface, and the rear surface, for example. Thus, a mobile phone can display data on not only its front surface but also its side surfaces, top surface, and rear surface, for example.

FIG. 18A illustrates an electronic appliance that is capable of receiving data via the Internet and displaying the data on the display unit 5230. An example of such an electronic appliance is a smartphone. For example, the user can check a created message on the display unit 5230 and send the created message to another device. As another example, the electronic appliance has a function of changing its display method in accordance with the illuminance of a usage environment. Thus, power consumption of the smartphone can be reduced. As another example, the smartphone can display an image on the display unit 5230 so as to be suitably used even in an environment under strong external light, e.g., outdoors in fine weather.

FIG. 18B illustrates an electronic appliance that can use a remote controller as the input unit 5240. An example of such an electronic appliance is a television system. For example, data received from a broadcast station or via the Internet can be displayed on the display unit 5230. The electronic appliance can take an image of the user with the sensor unit 5250 and transmit the image of the user. The electronic appliance can acquire a viewing history of the user and provide it to a cloud service. The electronic appliance can acquire recommendation data from a cloud service and display the data on the display unit 5230. A program or a moving image can be displayed on the basis of the recommendation data. As another example, the electronic appliance has a function of changing its display method in accordance with the illuminance of a usage environment. Accordingly, an image can be displayed on the display unit 5230 such that the electronic appliance can be suitably used even when irradiated with strong external light that enters the room from the outside in fine weather.

FIG. 18C illustrates an electronic appliance that is capable of receiving educational materials via the Internet and displaying them on the display unit 5230. An example of such an electronic appliance is a tablet computer. The user can input an assignment with the input unit 5240 and send it via the Internet. The user can obtain a corrected assignment or the evaluation from a cloud service and have it displayed on the display unit 5230. The user can select suitable educational materials on the basis of the evaluation and have them displayed.

For example, an image signal can be received from another electronic appliance and displayed on the display unit 5230. When the electronic appliance is placed on a stand or the like, the display unit 5230 can be used as a sub-display. As another example, an image can be displayed on the display unit 5230 such that the electronic appliance can be suitably used in an environment under strong external light, e.g., outdoors in fine weather.

FIG. 18D illustrates an electronic appliance including a plurality of display units 5230. An example of such an electronic appliance is a digital camera. For example, the display unit 5230 can display an image that the sensor unit 5250 is capturing. A captured image can be displayed on the display unit 5230. A captured image can be decorated using the input unit 5240. A message can be attached to a captured image. A captured image can be transmitted via the Internet. The electronic appliance has a function of changing shooting conditions in accordance with the illuminance of a usage environment. Accordingly, for example, a subject can be displayed on the display unit 5230 to be favorably viewed even in an environment under strong external light, e.g., outdoors in fine weather.

FIG. 18E illustrates an electronic appliance in which the electronic appliance of this embodiment is used as a master to control another electronic appliance used as a slave. An example of such an electronic appliance is a portable personal computer. For example, part of image data can be displayed on the display unit 5230 and another part of the image data can be displayed on a display unit of another electronic appliance. Image signals can be supplied. Data written from an input unit of another electronic appliance can be obtained with the communication unit 5290. Thus, a large display region can be utilized in the case of using a portable personal computer, for example.

FIG. 19A illustrates an electronic appliance including the sensing unit 5250 that senses an acceleration or a direction. An example of such an electronic appliance is a goggles-type electronic appliance. The sensor unit 5250 can supply data on the position of the user or the direction in which the user faces. The electronic appliance can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the display unit 5230, for example.

FIG. 19B illustrates an electronic appliance including an imaging device and the sensing unit 5250 that senses an acceleration or a direction. An example of such an electronic appliance is a glasses-type electronic appliance. The sensor unit 5250 can supply data on the position of the user or the direction in which the user faces. The electronic appliance can generate image data in accordance with the position of the user or the direction in which the user faces. Accordingly, the data can be shown together with a real-world scene, for example. Alternatively, an augmented reality image can be displayed on the glasses-type electronic appliance.

Note that this embodiment can be combined with any of the other embodiments in this specification as appropriate.

### Embodiment 6

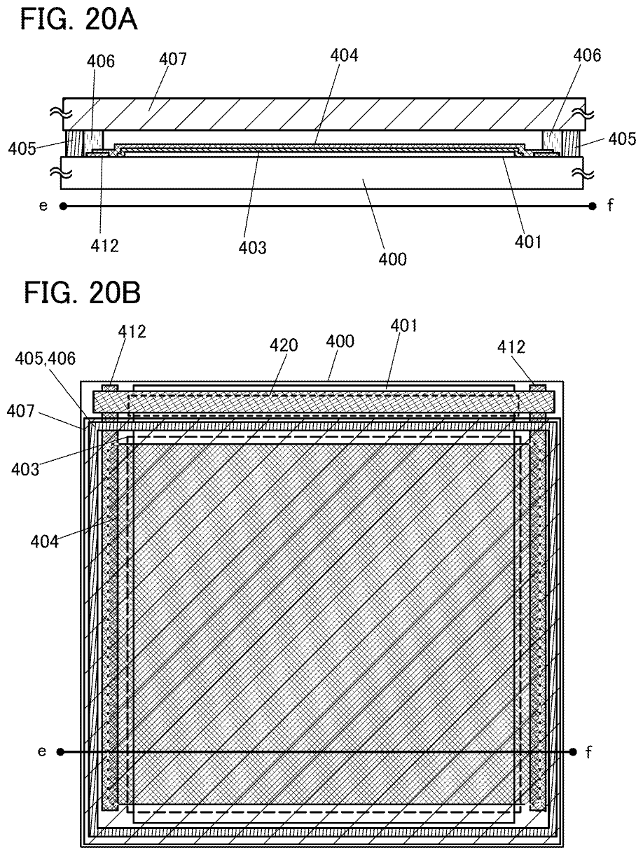

In this embodiment, a structure in which the light-emitting device described in Embodiment 2 is used in a lighting device will be described with reference to FIGS. 20A and 20B. FIG. 20A shows a cross section taken along the line e-f in a top view of the lighting device in FIG. 20B.

In the lighting device in this embodiment, a first electrode 401 is formed over a substrate 400 that is a support and has a light-transmitting property. The first electrode 401 corresponds to the first electrode 101 in Embodiment 2. When light is extracted from the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to, for example, the structure of the EL layer 103 in Embodiment 2 or the structure in which the EL layers 103a, 103b, and 103c and the charge-generation layers 106 (106a and 106b) are combined. Refer to the corresponding description for these structures.

The second electrode 404 is formed to cover the EL layer 403. The second electrode 404 corresponds to the second electrode 102 in Embodiment 2. The second electrode 404 is formed using a material having high reflectance when light is extracted from the first electrode 401 side. The second electrode 404 is connected to the pad 412 so that voltage is applied to the second electrode 404.

As described above, the lighting device described in this embodiment includes a light-emitting device including the first electrode 401, the EL layer 403, and the second electrode 404. Since the light-emitting device has high emission efficiency, the lighting device in this embodiment can have low power consumption.

The substrate 400 provided with the light-emitting device having the above structure and a sealing substrate 407 are fixed and sealed with sealing materials 405 and 406, whereby the lighting device is completed. It is possible to use only either the sealing material 405 or the sealing material 406. In addition, the inner sealing material 406 (not illustrated in FIG. 20B) can be mixed with a desiccant that enables moisture to be adsorbed, increasing the reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

### Embodiment 7

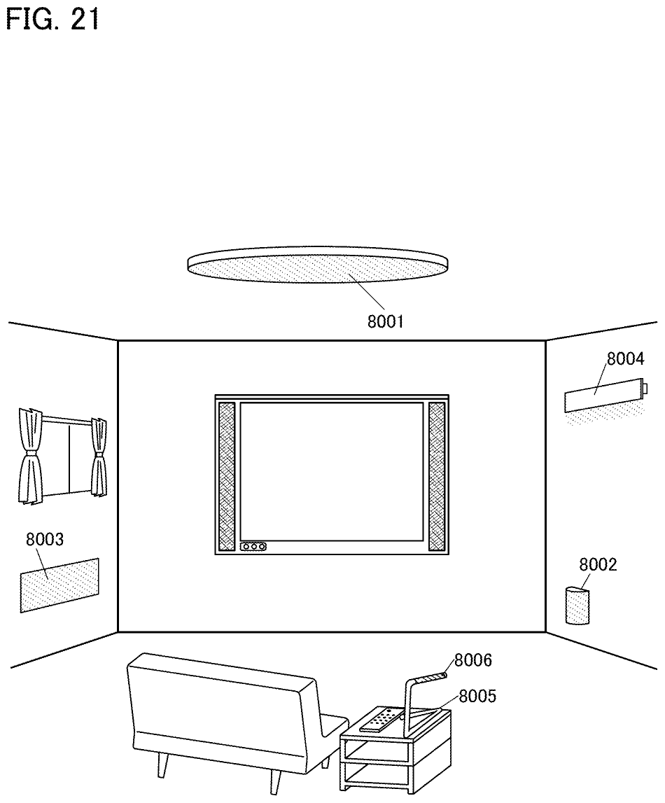

In this embodiment, application examples of lighting devices fabricated using the light-emitting apparatus of one embodiment of the present invention or the light-emitting device, which is part of the light-emitting apparatus, will be described with reference to FIG. 21.

A ceiling light 8001 can be used as an indoor lighting device. Examples of the ceiling light 8001 include a direct-mount light and an embedded light. Such lighting devices are fabricated using the light-emitting apparatus and a housing or a cover in combination. Application to a cord pendant light (light that is suspended from a ceiling by a cord) is also possible.

A foot light 8002 lights a floor so that safety on the floor can be improved. For example, it can be effectively used in a bedroom, on a staircase, and on a passage. In such cases, the size and shape of the foot light can be changed in accordance with the dimensions and structure of a room. The foot light can be a stationary lighting device fabricated using the light-emitting apparatus and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on a wall or a housing that has a curved surface.

A lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device, which is part of the light-emitting apparatus, is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in appropriate combination with any of the structures described in the other embodiments.

### Example 1

### Synthesis Example 1

In this example, a method for synthesizing 2-[3-{(3,5-di-tert-butyl)phenyl}-5-(pyrimidin-5-yl)phenyl]-4,6-diphenyl-1,3,5-triazine (abbreviation: mmtBuPh-mPmPTzn), which is the organic compound represented by Structural Formula (137) in Embodiment 1, is described. The structure of mmtBuPh-mPmPTzn is shown below.

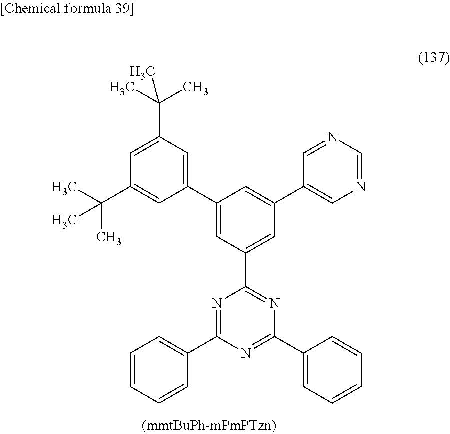 [SMILES: CC(C)(C)c1cc(-c2cc(-c3cncnc3)cc(-c3nc(-c4ccccc4)nc(-c4ccccc4)n3)c2)cc(C(C)(C)C)c1]

<Synthesis of mmtBuPh-mPmPTzn>

Into a three-neck flask were put 4.0 g (6.4 mmol) of 2-{3-(3,5-di-tert-butylphenyl)-5-(4,4,5,5-tetramethyl-1,3,2-dioxaborolan-2-yl)phenyl}-4,6-diphenyl-1,3,5-triazine, 0.93 g (5.8 mmol) of 5-bromopyrimidine, 32 mL of tetrahydrofuran (THF), and 9 mL of an aqueous solution of tripotassium phosphate (2 mol/L), and the mixture was degassed. To this mixture were added 13 mg (0.058 mmol) of palladium(II) acetate and 56 mg (0.12 mmol) of 2-dicyclohexylphosphino-2′,4′,6′-triisopropylbiphenyl (XPhos), and the mixture was heated at 65° C. for 22 hours to cause a reaction. After the reaction, the reacted solution was filtered and the filtrate was collected. The filtrate was subjected to extraction with toluene, and the organic layer was dried with magnesium sulfate. This mixture was gravity-filtered, and the obtained filtrate was concentrated to give a brown oil. This oil was purified by silica gel column chromatography with a developing solvent of ethyl acetate and hexane in a ratio of 1:5 to give a pale yellow solid. The obtained solid was purified by silica gel column chromatography with a developing solvent of ethyl acetate and toluene in a ratio of 1:5 to give a solid which was white to pale yellow in color. This solid was recrystallized with toluene and ethanol to give 2.9 g of a target white solid in a yield of 86%. The synthesis scheme is shown in Formula (a-1).

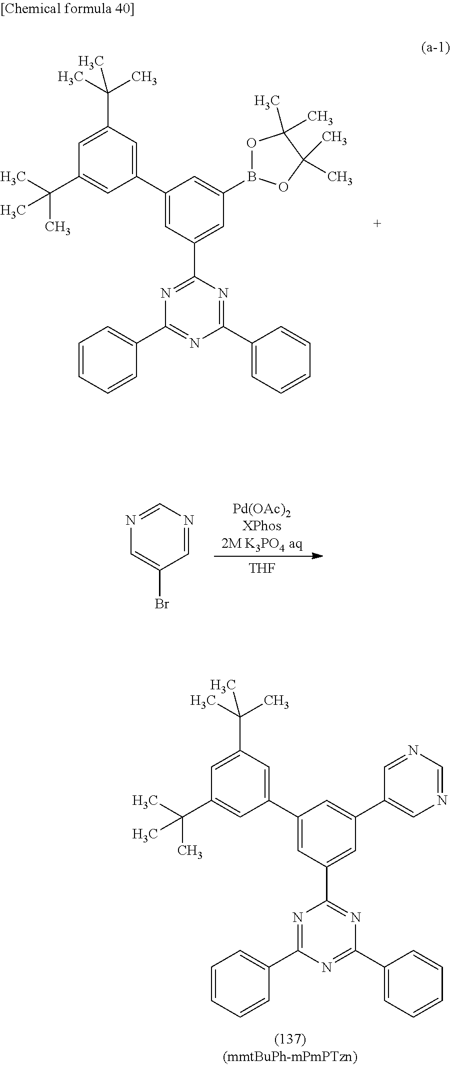

Then, 2.9 g of the obtained solid was purified by a train sublimation method. In the purification by sublimation, the solid was heated under a pressure of 6.0 Pa at 235° C. for 17 hours, at 240° C. for 7.5 hours, and then at 245° C. for 16.5 hours while an argon gas was made to flow. After the purification by sublimation, 2.7 g of a target white solid was obtained at a collection rate of 92%.

Analysis results by nuclear magnetic resonance (¹H-NMR) spectroscopy of the obtained white solid are shown below. These results revealed that mmtBuPh-mPmPTzn, which is the organic compound of one embodiment of the present invention represented by Structural Formula (137), was obtained in this example.

¹H NMR (CDCl₃, 300 MHz): δ=1.45 (s, 18H), 7.57-7.67 (m, 9H), 7.99 (t, J=2.0 Hz, 1H), 8.80 (dd, J=7.7 Hz, 1.7 Hz, 4H), 8.96 (t, J=1.7 Hz, 1H), 9.09 (t, J=1.5 Hz, 1H), 9.19 (s, 2H), 9.32 (s, 1H).

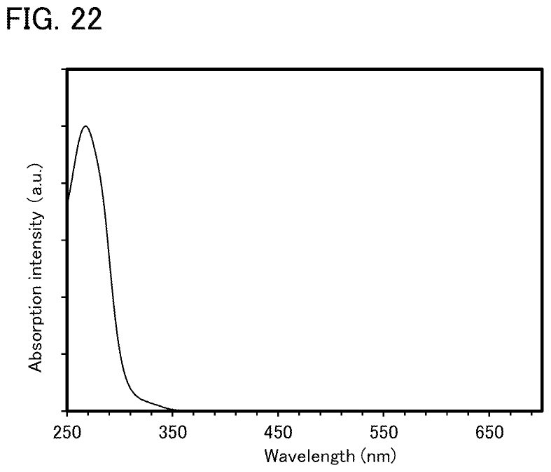

Next, an ultraviolet-visible absorption spectrum (hereinafter simply referred to as absorption spectrum) of the obtained organic compound in dichloromethane was measured. The absorption spectrum was measured at room temperature using a V550 ultraviolet-visible spectrophotometer manufactured by JASCO Corporation. A quartz cell was used as the measurement cell. FIG. 22 shows measurement results of the absorption spectrum. The horizontal axis represents wavelength and the vertical axis represents absorption intensity. The absorption spectrum in FIG. 22 is a result obtained by subtraction of a measured absorption spectrum of dichloromethane alone in a quartz cell from a measured absorption spectrum of the dichloromethane solution in a quartz cell.

As shown in FIG. 22, an absorption peak was observed at 268 nm, revealing that no absorption was shown in the visible region range of 440 nm to 700 nm.

Next, the obtained organic compound was subjected to mass spectrometry (MS) analysis by liquid chromatography-mass spectrometry (LC-MS).

In the LC/MS analysis, liquid chromatography (LC) separation was performed with UltiMate 3000 manufactured by Thermo Fisher Scientific K.K., and mass spectrometry (MS) was performed with Q Exactive manufactured by Thermo Fisher Scientific K.K.

In the LC separation, a given column was used at a column temperature of 40° C., and solution sending was performed in such a manner that an appropriate solvent was selected, the sample was prepared by dissolving the organic compound in an organic solvent at an arbitrary concentration, and the injection amount was 5.0 μL.

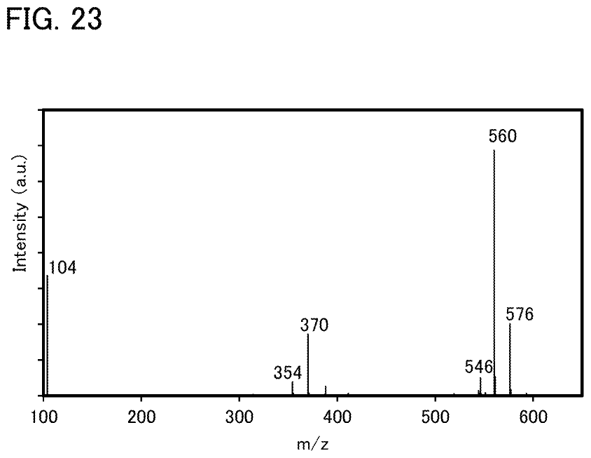

By a parallel reaction monitoring (PRM) method, MS² measurement of m/z=575.30 corresponding to the exact mass of mmtBuPh-mPmPTzn was performed. For setting of the PRM, the mass range of a target ion was set to m/z=575.30±2.0 (isolation window=4) and detection was performed in a positive mode. The measurement was performed with energy (normalized collision energy: NCE) for accelerating a target ion in a collision cell set to 60. The obtained MS spectrum is shown in FIG. 23.

Fragment ions at m/z of 104.05 and m/z of 370.23 were observed. These fragment ions are each probably a fragment formed of one substituent bonded to the triazine and carbon and nitrogen derived from the triazine. For example, the fragment ion at m/z of 104.05 is probably a fragment in which one carbon atom and one nitrogen atom each derived from the triazine are bonded to a phenyl group. The fragment ion at m/z of 370.23 is probably a fragment in which one carbon atom and one nitrogen atom each derived from the triazine are bonded to a substituent other than a phenyl group. These fragments can each be regarded as a feature of a compound having a triazine skeleton.

From the results in FIG. 23, it was confirmed that the target substance mmtBuPh-mPmPTzn was obtained.

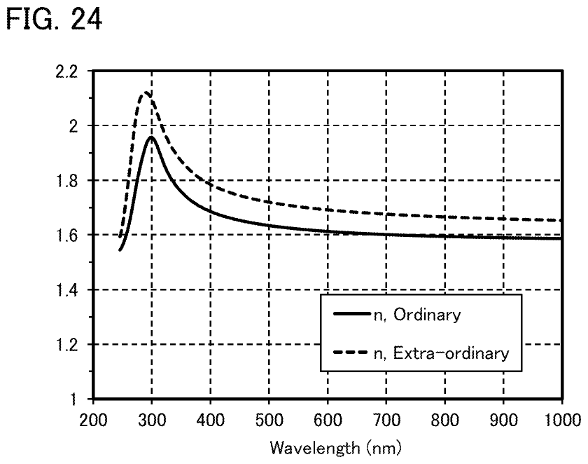

FIG. 24 shows the results of measuring the refractive index of mmtBuPh-mPmPTzn with an M-2000U spectroscopic ellipsometer manufactured by J.A. Woollam Japan Corp. A film used for the measurement was formed to a thickness of approximately 50 nm with the material of each layer over a quartz substrate by a vacuum evaporation method. Note that a refractive index for an ordinary ray, n, Ordinary, and a refractive index for an extraordinary ray, n, Extra-ordinary, are shown in FIG. 24.

FIG. 24 shows that mmtBuPh-mPmPTzn is a material with a low refractive index: the ordinary refractive index of mmtBuPh-mPmPTzn in the entire blue emission region (from 455 nm to 465 nm) is 1.65, which is in the range of 1.50 to 1.75, and the ordinary refractive index of mmtBuPh-mPmPTzn at a wavelength of 633 nm is 1.61, which is in the range of 1.45 to 1.70.

### Example 2

### Synthesis Example 2

In this example, a method for synthesizing 2-[3-{(3,5-di-tert-butyl)phenyl}-5-(pyrazin-2-yl)phenyl]-4,6-diphenyl-1,3,5-triazine (abbreviation: mmtBuPh-mPrPTzn), which is the organic compound represented by Structural Formula (154) in Embodiment 1, is described. The structure of mmtBuPh-mPrPTzn is shown below.

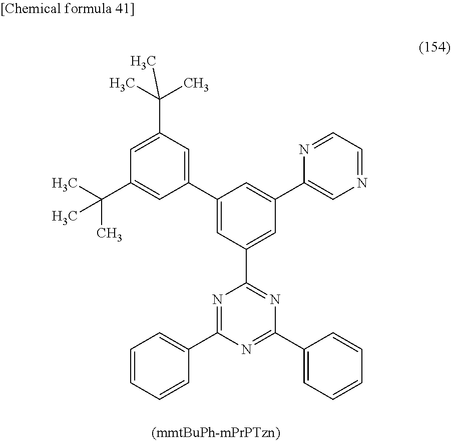 [SMILES: CC(C)(C)c1cc(-c2cc(-c3cnccn3)cc(-c3nc(-c4ccccc4)nc(-c4ccccc4)n3)c2)cc(C(C)(C)C)c1]

<Synthesis of mmtBuPh-mPrPTzn>

Into a three-neck flask were put 4.0 g (6.4 mmol) of 2-{3-(3,5-di-tert-butylphenyl)-5-(4,4,5,5-tetramethyl-1,3,2-dioxaborolan-2-yl)phenyl}-4,6-diphenyl-1,3,5-triazine, 0.67 g (5.8 mmol) of chloropyrazine, 1.6 g (12 mmol) of potassium carbonate, 30 mL of tetrahydrofuran, and 15 mL of water, and the mixture was degassed. To this mixture was added 0.13 g (0.12 mmol) of tetrakis(triphenylphosphine)palladium(0), and the mixture was heated at 65° C. for 14 hours to cause a reaction. After the reaction, extraction was performed with toluene, and the organic layer was dried with magnesium sulfate. This mixture was gravity-filtered, and the obtained filtrate was concentrated to give a brown solid. This solid was purified by silica gel column chromatography while the polarity of a developing solvent was sequentially changed from only toluene to a mixed solvent of toluene and ethyl acetate in a ratio of 100:1 and to 10:1 to give a solid which was white to pale yellow in color. The obtained solid was purified by silica gel column chromatography with a developing solvent of toluene and ethyl acetate in a ratio of 50:1 to give a white solid. This solid was recrystallized with toluene and ethanol to give 3.0 g of a target white solid in a yield of 90%. The synthesis scheme is shown in Formula (b-1).

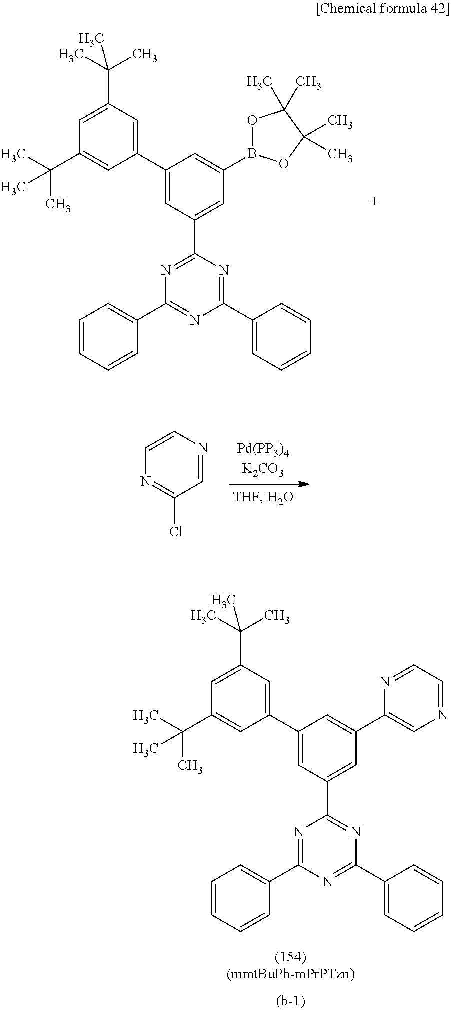

Then, 3.0 g of the obtained solid was purified by a train sublimation method. In the purification by sublimation, the solid was heated under a pressure of 5.6 Pa at 240° C. for 23 hours and then at 245° C. for 23 hours while an argon gas was made to flow. After the purification by sublimation, 2.8 g of a target white solid was obtained at a collection rate of 94%.

Analysis results by nuclear magnetic resonance (¹H-NMR) spectroscopy of the obtained white solid are shown below. These results revealed that mmtBuPh-mPrPTzn, which is the organic compound of one embodiment of the present invention represented by Structural Formula (154), was obtained in this example.

¹H NMR (CDCl₃, 300 MHz): δ=1.45 (s, 18H), 7.55-7.66 (m, 9H), 8.48 (t, J=1.7 Hz, 1H), 8.62 (d, J=2.7 Hz, 1H), 8.76 (t, J=2.1 Hz, 1H), 8.82 (dd, J=8.0 Hz, 1.7 Hz, 4H), 9.11 (t, J=1.7 Hz, 1H), 9.29 (d, J=1.5 Hz, 1H), 9.35 (t, J=1.7 Hz, 1H).

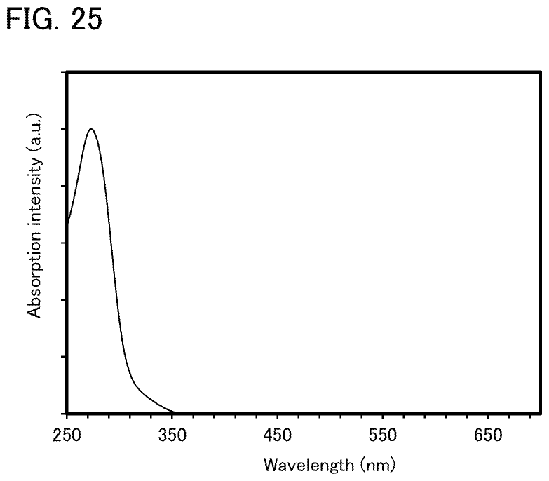

Next, an absorption spectrum of the obtained organic compound in dichloromethane was measured. The absorption spectrum was measured at room temperature using a V550 ultraviolet-visible spectrophotometer manufactured by JASCO Corporation. A quartz cell was used as the measurement cell. FIG. 25 shows measurement results of the absorption spectrum. The horizontal axis represents wavelength and the vertical axis represents absorption intensity. The absorption spectrum in FIG. 25 is a result obtained by subtraction of a measured absorption spectrum of dichloromethane alone in a quartz cell from a measured absorption spectrum of the dichloromethane solution in a quartz cell.

Next, the obtained organic compound was subjected to mass spectrometry (MS) analysis by liquid chromatography-mass spectrometry (LC-MS).

In the LC/MS analysis, liquid chromatography (LC) separation was performed with UltiMate 3000 manufactured by Thermo Fisher Scientific K.K., and mass spectrometry (MS) was performed with Q Exactive manufactured by Thermo Fisher Scientific K.K.

In the LC separation, a given column was used at a column temperature of 40° C., and solution sending was performed in such a manner that an appropriate solvent was selected, the sample was prepared by dissolving mmtBuPh-mPrPTzn in an organic solvent at an arbitrary concentration, and the injection amount was 5.0 μL.

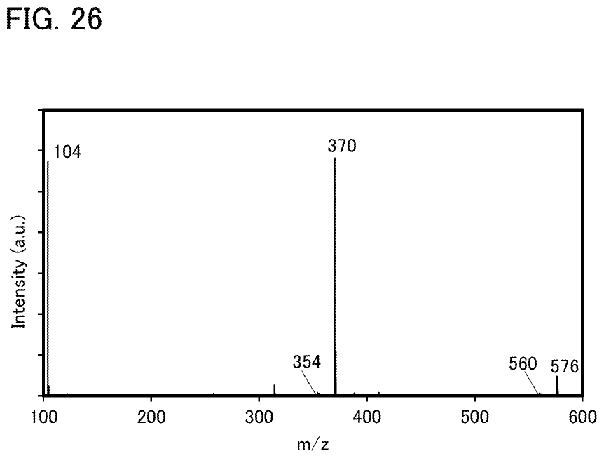

By a PRM method, MS² measurement of m/z=575.30 corresponding to the exact mass of mmtBuPh-mPrPTzn was performed. For setting of the PRM, the mass range of a target ion was set to m/z=575.30±2.0 (isolation window=4) and detection was performed in a positive mode. The measurement was performed with energy (normalized collision energy: NCE) for accelerating a target ion in a collision cell set to 50. The MS spectrum obtained by the MS² measurement is shown in FIG. 26.

Fragment ions at m/z of 104.05 and m/z of 370.23 were observed. These fragment ions are each probably a fragment formed of one substituent bonded to the triazine and carbon and nitrogen derived from the triazine. For example, the fragment ion at m/z of 104.05 is probably a fragment in which one carbon atom and one nitrogen atom each derived from the triazine are bonded to a phenyl group. The fragment ion at m/z of 370.23 is probably a fragment in which one carbon atom and one nitrogen atom each derived from the triazine are bonded to a substituent other than a phenyl group. These fragments can each be regarded as a feature of a compound having a triazine skeleton.

From the results in FIG. 26, it was confirmed that the target substance mmtBuPh-mPrPTzn was obtained.

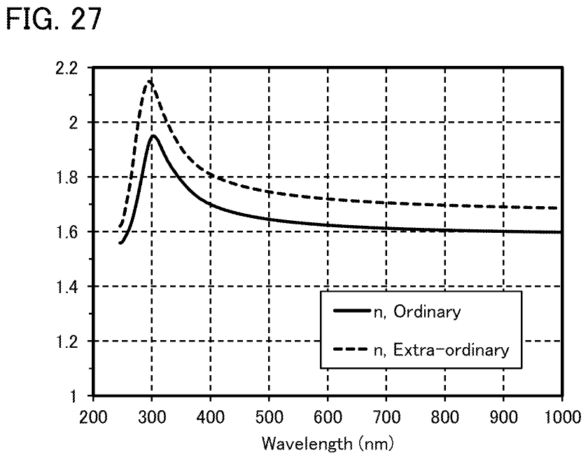

FIG. 27 shows the results of measuring the refractive index of mmtBuPh-mPrPTzn with an M-2000U spectroscopic ellipsometer manufactured by J.A. Woollam Japan Corp. A film used for the measurement was formed to a thickness of approximately 50 nm with the material of each layer over a quartz substrate by a vacuum evaporation method. Note that a refractive index for an ordinary ray, n, Ordinary, and a refractive index for an extraordinary ray, n, Extra-ordinary, are shown in FIG. 27.

FIG. 27 shows that mmtBuPh-mPrPTzn is a material with a low refractive index: the ordinary refractive index of mmtBuPh-mPrPTzn in the entire blue emission region (from 455 nm to 465 nm) is 1.66, which is in the range of 1.50 to 1.75, and the ordinary refractive index of mmtBuPh-mPrPTzn at a wavelength of 633 nm is 1.62, which is in the range of 1.45 to 1.70.

### Example 3

In this example, a light-emitting device 1 of one embodiment of the present invention described in the above embodiment and a comparative light-emitting device 1 are described. Structural formulae of organic compounds used in this example are shown below.

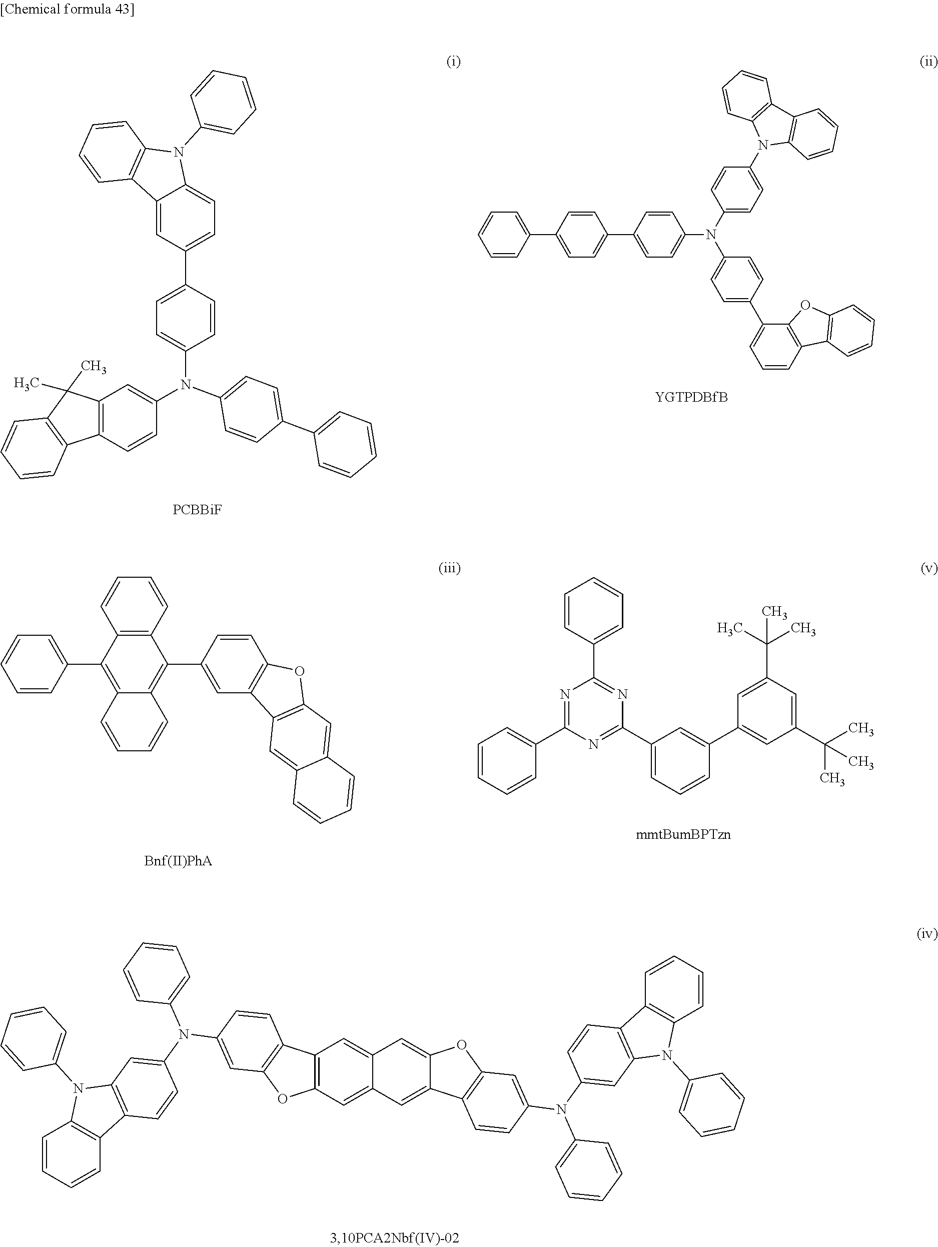

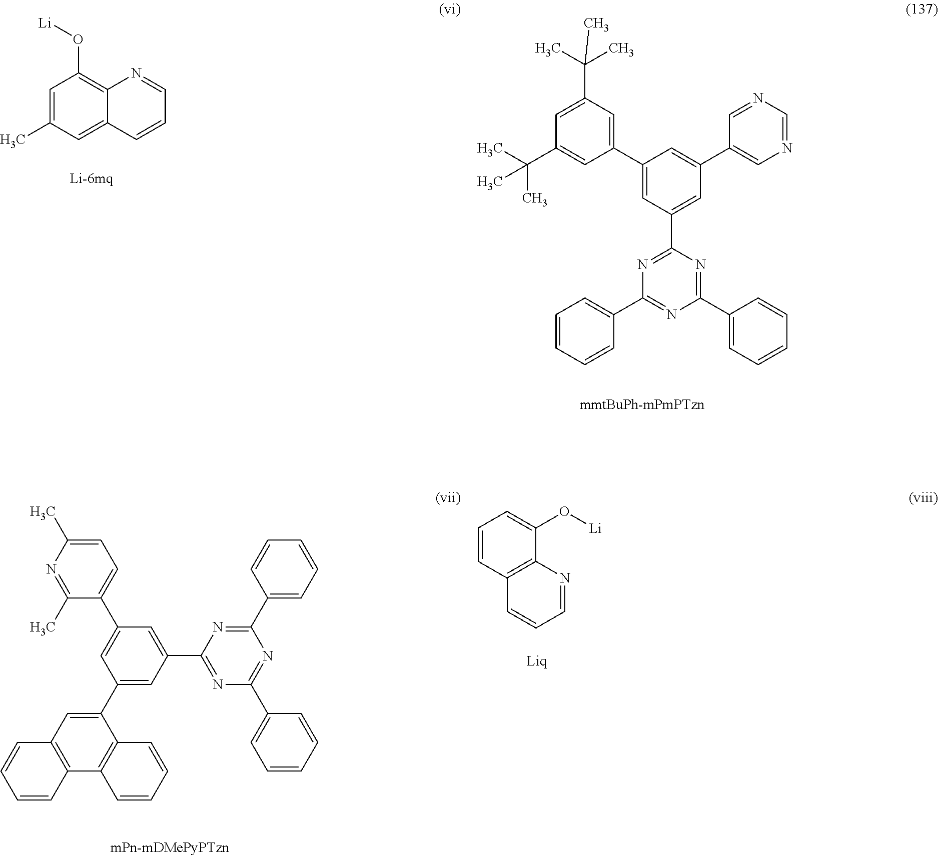

### (Fabrication Method of Light-Emitting Device 1)

First, as a transparent electrode, indium tin oxide containing silicon oxide (ITSO) was deposited over a glass substrate to a thickness of 70 nm by a sputtering method, whereby the first electrode 101 was formed. The electrode area was 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was allowed to cool down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) and a fluorine-containing electron acceptor material with a molecular weight of 672 (OCHD-003) were deposited by co-evaporation to a thickness of 10 nm such that the weight ratio of PCBBiF to OCHD-003 was 1:0.05, whereby the hole-injection layer 111 was formed.

Over the hole-injection layer 111, PCBBiF was deposited by evaporation to a thickness of 20 nm, whereby the hole-transport layer 112 was formed.

Next, over the hole-transport layer 112, N-[4-(9H-carbazol-9-yl)phenyl]-N-[4-(4-dibenzofuranyl)phenyl]-[1,1′:4′,1″-terphenyl]-4-amine (abbreviation: YGTPDBfB) represented by Structural Formula (ii) was deposited by evaporation to a thickness of 10 nm to form an electron-blocking layer.

Then, 2-(10-phenyl-9-anthracenyl)-benzo[b]naphtho[2,3-d]furan (abbreviation: Bnf(II)PhA) represented by Structural Formula (iii) and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, 2-{(3′,5′-di-tert-butyl)-1,1′-biphenyl-3-yl}-4,6-diphenyl-1,3,5-triazine (abbreviation: mmtBumBPTzn) represented by Structural Formula (v) was deposited by evaporation to a thickness of 10 nm to form a hole-blocking layer. Then, 2-[3-{(3,5-di-tert-butyl)phenyl}-5-(pyrimidin-5-yl)phenyl]-4,6-diphenyl-1,3,5-triazine (abbreviation: mmtBuPh-mPmPTzn) (Structural Formula (100)) which is one embodiment of the present invention described in Example 1 and 6-methyl-8-quinolinolato-lithium (abbreviation: Li-6mq) represented by Structural Formula (vi) were deposited by co-evaporation to a thickness of 20 nm such that the weight ratio of mmtBuPh-mPmPTzn to Li-6mq was 1:1, whereby the electron-transport layer 114 was formed.

After the formation of the electron-transport layer 114, LiF was deposited to a thickness of 1 nm to form the electron-injection layer 115.

Lastly, aluminum (Al) was deposited by evaporation to a thickness of 200 nm to form the second electrode 102, whereby the light-emitting device 1 was fabricated.

### (Fabrication Method of Comparative Light-Emitting Device 1)

The comparative light-emitting device 1 was fabricated in the same manner as the light-emitting device 1 except that 2-[3-(2,6-dimethylpyridin-3-yl)-5-(9-phenanthryl)phenyl]-4,6-diphenyl-1,3,5-triazine (abbreviation: mPn-mDMePyPTzn) represented by Structural Formula (vii) was used instead of mmtBuPh-mPmPTzn for the electron-transport layer 114; and 8-quinolinolato-lithium (abbreviation: Liq) represented by Structural Formula (viii) was used instead of Li-6mq.

The structures of the light-emitting device 1 and the comparative light-emitting device 1 are listed in Table 1 below.

TABLE 1

| | | | Comparative |
|---|---|---|---|
| | Thickness | Light-emitting device 1 | light-emitting device 1 |
| Second electrode | 200 nm | Al | |
| Electron-injection | 1 nm | LiF | |
| layer | | | |
| Electron-transport | 20 nm | mmtBuPh-mPmPTzn:Li-6mq | mPn-mDMePyPTzn:Liq |
| layer | | (1:1) | (1:1) |

| | | |
|---|---|---|
| Hole-blocking layer | 10 nm | mmtBumBPTzn |
| Light-emitting layer | 25 nm | Bnf(II)PhA:3,10PCA2Nbf(IV)-02 |
| | | (1:0.015) |
| Electron-blocking | 10 nm | YGTPDBfB |
| layer | | |
| Hole-transport layer | 20 nm | PCBBiF |
| Hole-injection layer | 10 nm | PCBBiF:OCHD-003 |
| | | (1:0.05) |
| First electrode | 70 nm | ITSO |

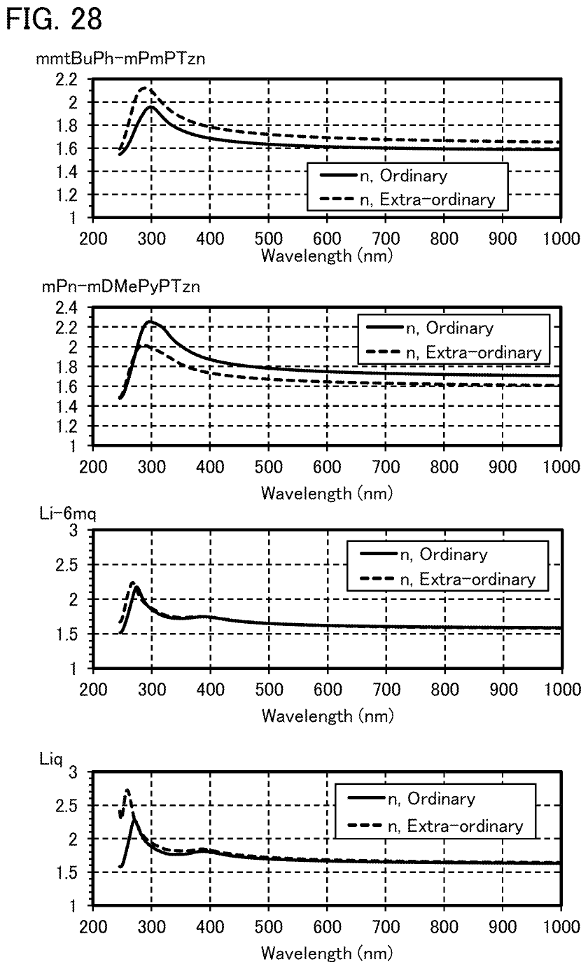

FIG. 28 shows the refractive indices of mmtBuPh-mPmPTzn, mPn-mDMePyPTzn, Li-6mq, and Liq, and Table 2 shows the refractive indices at a wavelength of 456 nm. The refractive indices were measured with an M-2000U spectroscopic ellipsometer manufactured by J.A. Woollam Japan Corp. As a sample used for the measurement, a film was formed to a thickness of approximately 50 nm with the material of each layer over a quartz substrate by a vacuum evaporation method. Note that a refractive index for an ordinary ray, n, Ordinary, and a refractive index for an extraordinary ray, n, Extra-ordinary, are shown in FIG. 28.

TABLE 2

| | Ordinary refractive index |
|---|---|
| | (n, Ordinary) @456 nm |
| mmtBuPh-mPmPTzn | 1.61 |
| mPn-mDMePyPTzn | 1.81 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

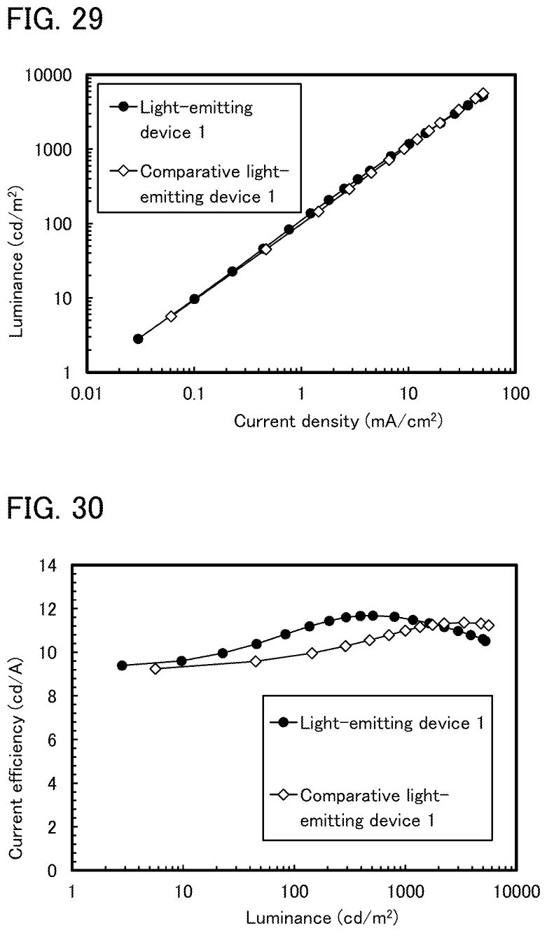

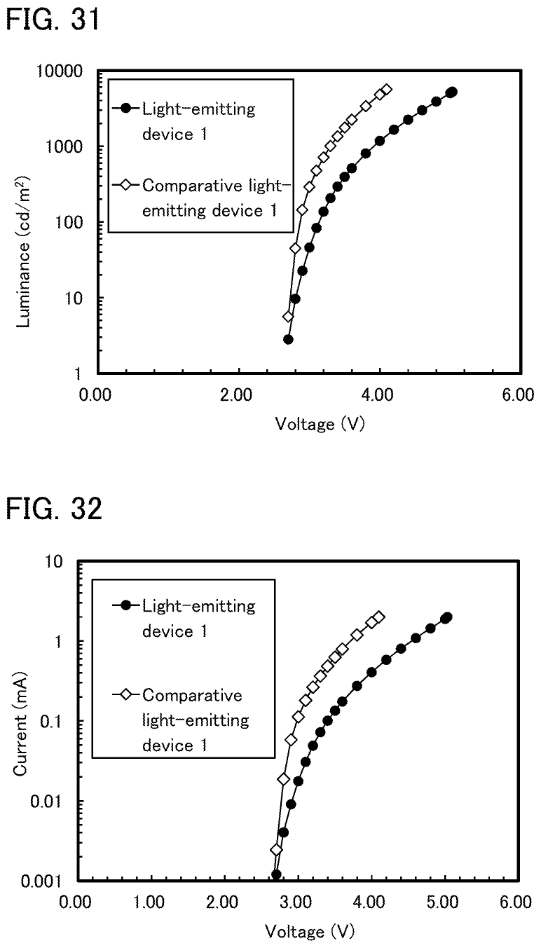

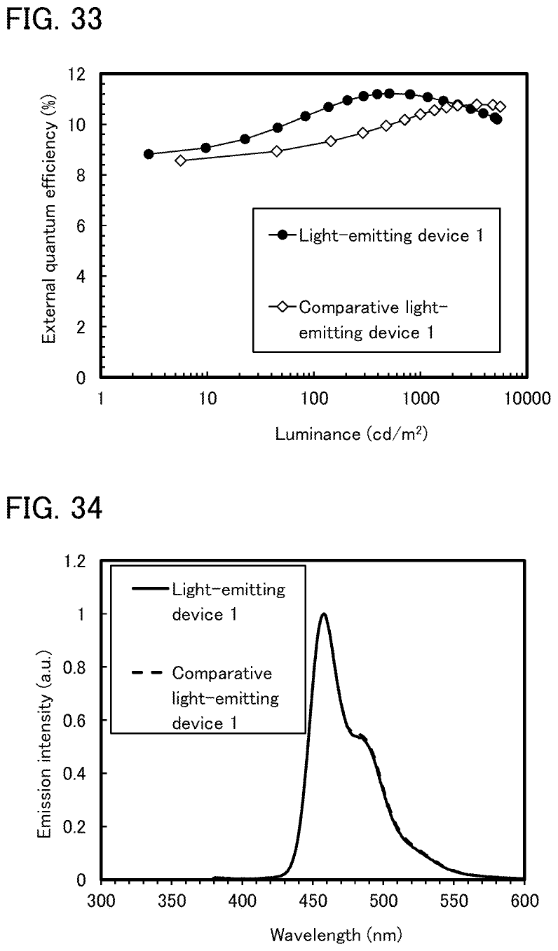

FIG. 29 shows the luminance-current density characteristics of the light-emitting device 1 and the comparative light-emitting device 1. FIG. 30 shows the current efficiency-luminance characteristics thereof. FIG. 31 shows the luminance-voltage characteristics thereof. FIG. 32 shows the current-voltage characteristics thereof. FIG. 33 shows the external quantum efficiency-luminance characteristics thereof. FIG. 34 shows the emission spectra thereof. Table 3 shows the main characteristics of the light-emitting device 1 and the comparative light-emitting device 1 at a luminance of about 1000 cd/m². The luminance, CIE chromaticity, and emission spectra were measured at normal temperature with an SR-UL1R spectroradiometer manufactured by TOPCON TECHNOHOUSE CORPORATION.

TABLE 3

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 4.0 | 0.41 | 10.2 | 0.13 | 0.12 | 11.5 | 11.1 |
| device 1 | | | | | | | |
| Comparative light- | 3.3 | 0.36 | 9.1 | 0.13 | 0.13 | 11.5 | 10.4 |
| emitting device 1 | | | | | | | |

The results in FIG. 29 to FIG. 34 and Table 3 revealed that the light-emitting device 1 fabricated using the low refractive index material of one embodiment of the present invention is an EL device having substantially the same emission spectrum as the comparative light-emitting device 1 and having higher external quantum efficiency than the comparative light-emitting device 1.

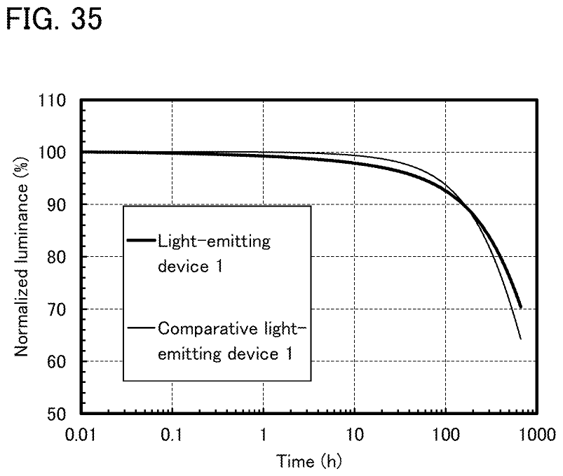

Next, reliability tests were performed on the light-emitting devices. FIG. 35 shows the results of the reliability tests of the light-emitting device 1 and the comparative light-emitting device 1. In FIG. 35, the vertical axis represents normalized luminance (%) with an initial luminance of 100%, and the horizontal axis represents driving time (h) of the devices. As the reliability tests, driving tests at a constant current density of 50 mA/cm² were performed on the light-emitting devices.

The results in FIG. 35 showed that the light-emitting device 1 including the low refractive index material of one embodiment of the present invention had favorable reliability comparable to that of the comparative light-emitting device. Thus, one embodiment of the present invention is suitable for a light-emitting device used for a display.

### Example 4

In this example, a light-emitting device 2 of one embodiment of the present invention and a comparative light-emitting device 2 are described. Structural formulae of organic compounds used in this example are shown below.

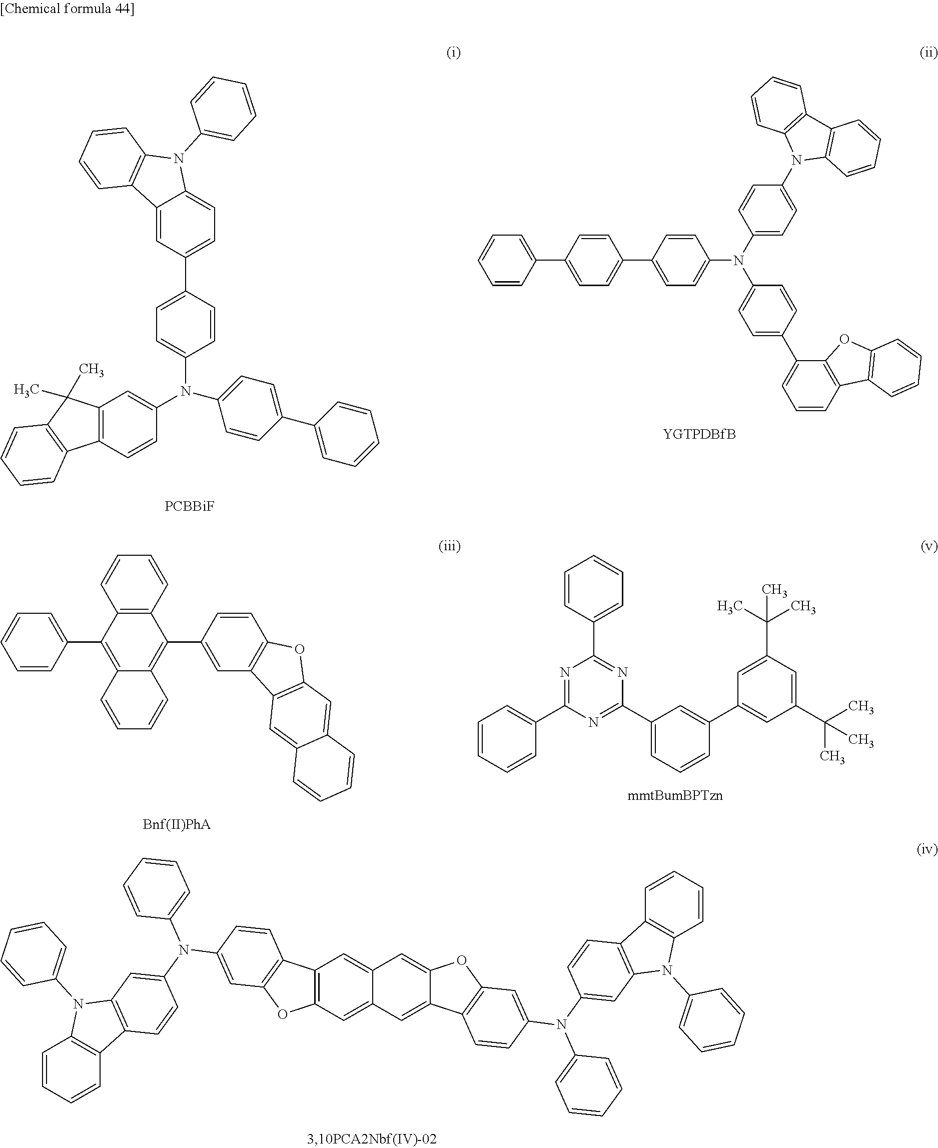

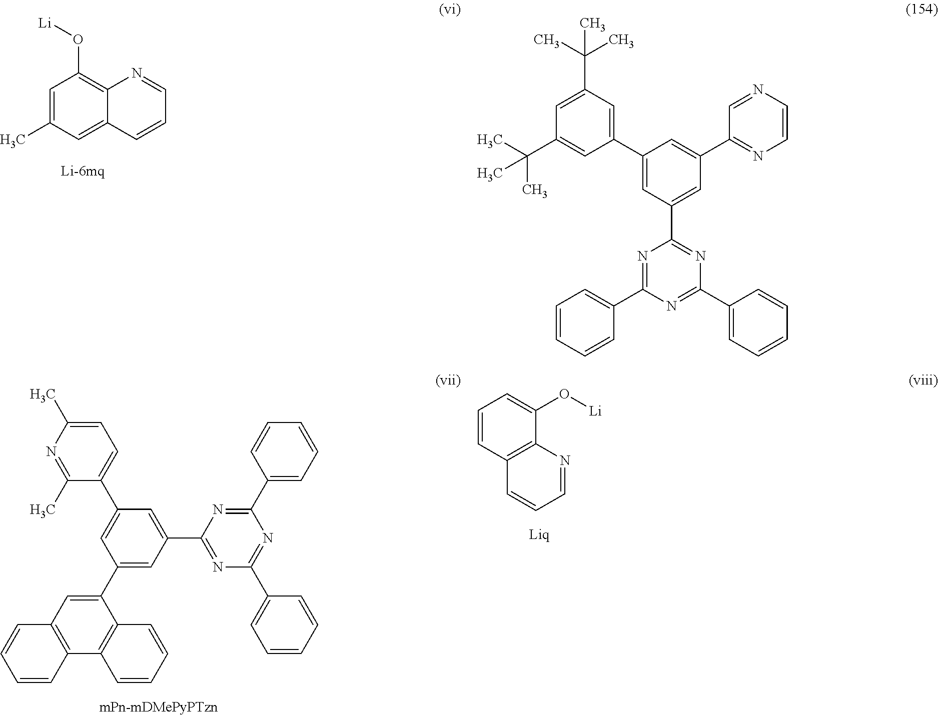

### (Fabrication Method of Light-Emitting Device 2)

First, as a transparent electrode, ITSO was deposited over a glass substrate to a thickness of 70 nm by a sputtering method, whereby the first electrode 101 was formed. The electrode area was 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was allowed to cool down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, PCBBiF represented by Structural Formula (i) and a fluorine-containing electron acceptor material with a molecular weight of 672 (OCHD-003) were deposited by co-evaporation to a thickness of 10 nm such that the weight ratio of PCBBiF to OCHD-003 was 1:0.05, whereby the hole-injection layer 111 was formed.

Over the hole-injection layer 111, PCBBiF was deposited by evaporation to a thickness of 20 nm, whereby the hole-transport layer 112 was formed.

Next, over the hole-transport layer 112, YGTPDBfB represented by Structural Formula (ii) was deposited by evaporation to a thickness of 10 nm to form an electron-blocking layer.

Then, Bnf(II)PhA represented by Structural Formula (iii) and 3,10PCA2Nbf(IV)-02 represented by Structural Formula (iv) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, mmtBumBPTzn represented by Structural Formula (v) was deposited by evaporation to a thickness of 10 nm to form a hole-blocking layer. Then, 2-[3-{(3,5-di-tert-butyl)phenyl}-5-(pyrazin-2-yl)phenyl]-4,6-diphenyl-1,3,5-triazine (abbreviation: mmtBuPh-mPrPTzn) (Structural Formula (101)) which is one embodiment of the present invention described in Example 2 and Li-6mq represented by Structural Formula (vi) were deposited by co-evaporation to a thickness of 20 nm such that the weight ratio of mmtBuPh-mPrPTzn to Li-6mq was 1:1, whereby the electron-transport layer 114 was formed.

After the formation of the electron-transport layer 114, LiF was deposited to a thickness of 1 nm to form the electron-injection layer 115.

Lastly, aluminum (Al) was deposited by evaporation to a thickness of 200 nm to form the second electrode 102, whereby the light-emitting device 1 was fabricated.

### (Fabrication Method of Comparative Light-Emitting Device 2)

The comparative light-emitting device 2 was fabricated in the same manner as the light-emitting device 2 except that mPn-mDMePyPTzn represented by Structural Formula (vii) was used instead of mmtBuPh-mPrPTzn for the electron-transport layer 114; and Liq represented by Structural Formula (viii) was used instead of Li-6mq.

The structures of the light-emitting device 2 and the comparative light-emitting device 2 are listed in Table 4 below.

TABLE 4

| | | | Comparative |
|---|---|---|---|
| | Thickness | Light-emitting device 2 | light-emitting device 2 |
| Second electrode | 200 nm | Al | |
| Electron-injection | 1 nm | LiF | |
| layer | | | |
| Electron-transport | 20 nm | mmtBuPh-mPrPTzn:Li-6mq | mPn-mDMePyPTzn:Liq |
| layer | | (1:1) | (1:1) |

| | | |
|---|---|---|
| Hole-blocking | 10 nm | mmtBumBPTzn |
| layer | | |
| Light-emitting | 25 nm | Bnf(II)PhA:3,10PCA2Nbf(IV)-02 |
| layer | | (1:0.015) |
| Electron-blocking | 10 nm | YGTPDBfB |
| layer | | |
| Hole-transport | 20 nm | PCBBiF |
| layer | | |
| Hole-injection | 10 nm | PCBBiF:OCHD-003 |
| layer | | (1:0.05) |
| First electrode | 70 nm | ITSO |

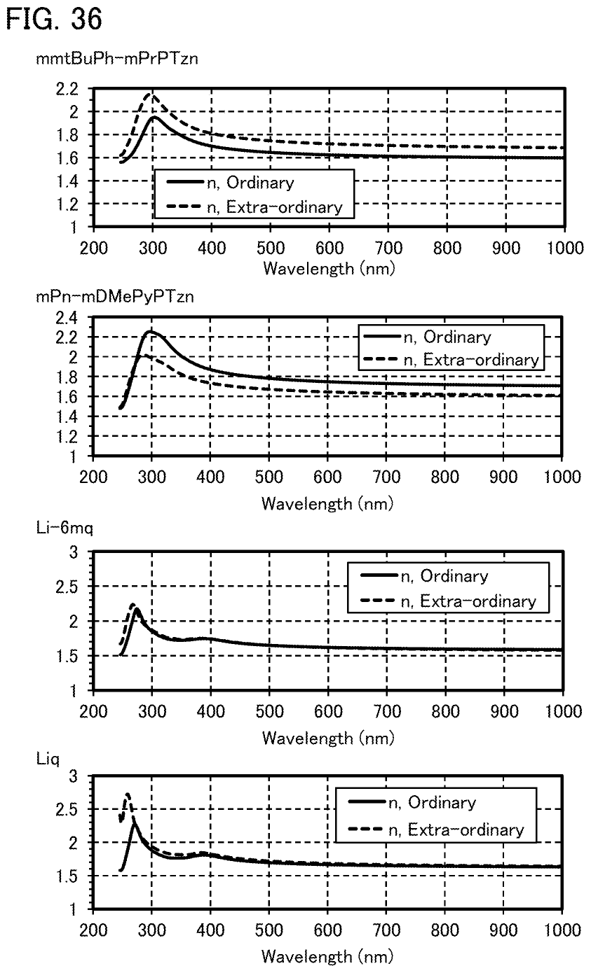

FIG. 36 shows the refractive indices of mmtBuPh-mPrPTzn, mPn-mDMePyPTzn, Li-6mq, and Liq, and Table 5 shows the refractive indices at a wavelength of 456 nm. The refractive indices were measured with an M-2000U spectroscopic ellipsometer manufactured by J.A. Woollam Japan Corp. As a sample used for the measurement, a film was formed to a thickness of approximately 50 nm with the material of each layer over a quartz substrate by a vacuum evaporation method. Note that a refractive index for an ordinary ray, n, Ordinary, and a refractive index for an extraordinary ray, n, Extra-ordinary, are shown in FIG. 36.

TABLE 5

| | Ordinary refractive index |
|---|---|
| | (n , Ordinary) @456 nm |
| mmtBuPh-mPrPTzn | 1.62 |
| mPn-mDMePyPTzn | 1.81 |
| Li-6mq | 1.67 |
| Liq | 1.72 |

The above light-emitting device and comparative light-emitting device were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

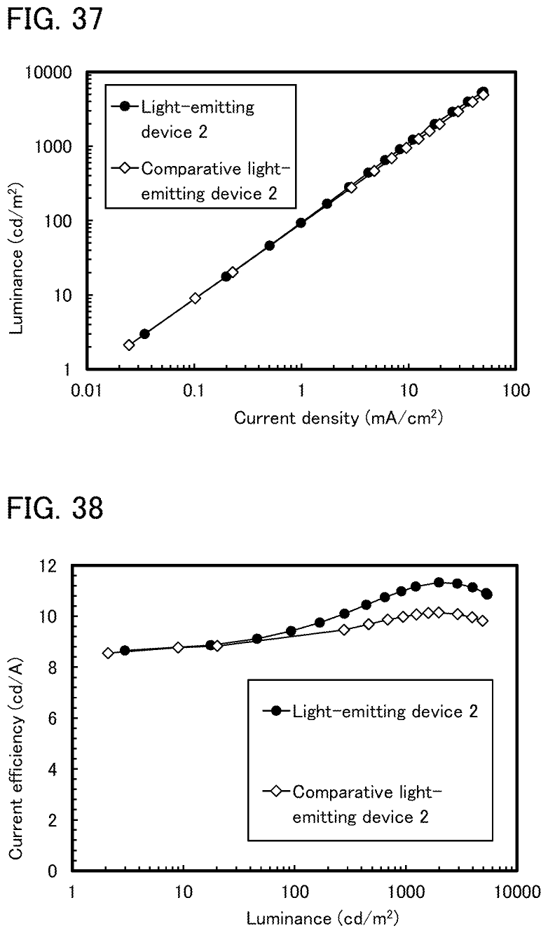

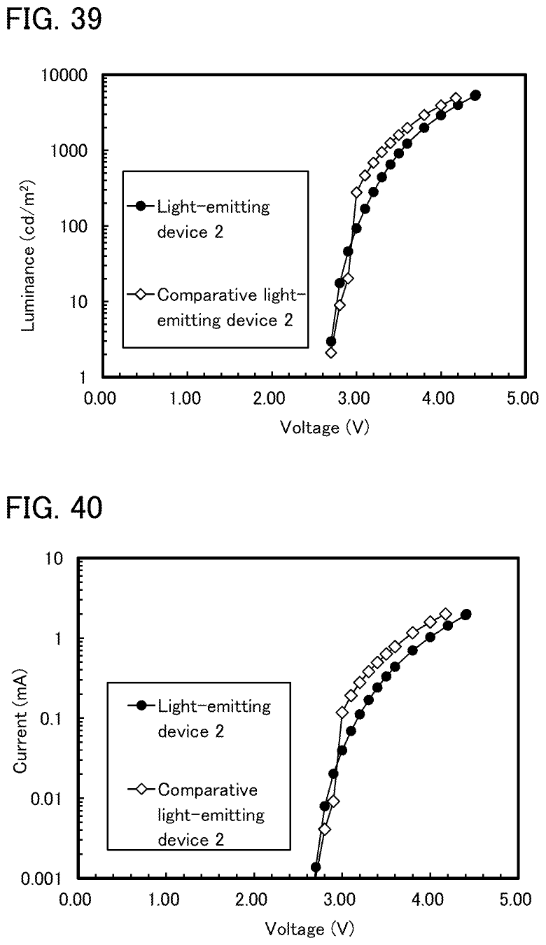

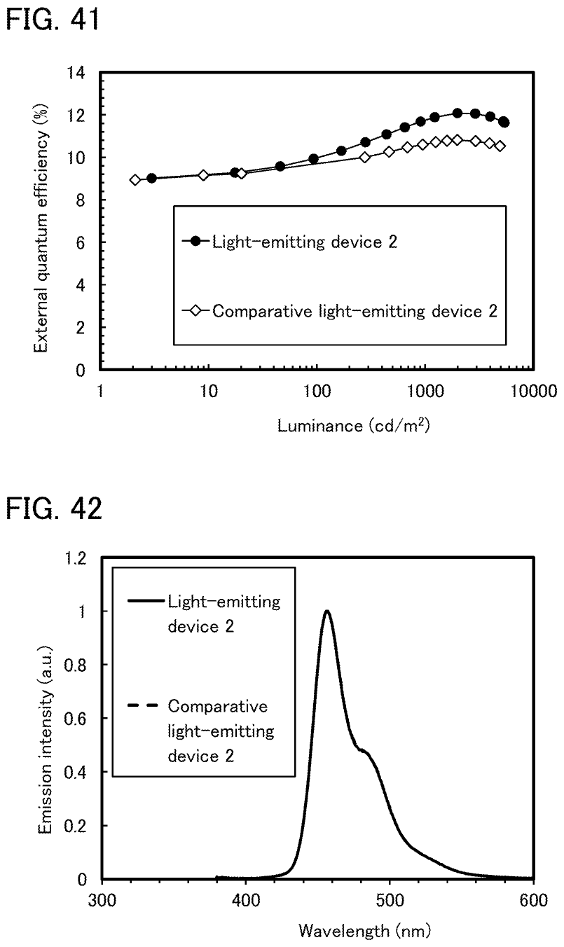

FIG. 37 shows the luminance-current density characteristics of the light-emitting device 2 and the comparative light-emitting device 2. FIG. 38 shows the current efficiency-luminance characteristics thereof. FIG. 39 shows the luminance-voltage characteristics thereof. FIG. 40 shows the current-voltage characteristics thereof. FIG. 41 shows the external quantum efficiency-luminance characteristics thereof. FIG. 42 shows the emission spectra thereof. Table 6 shows the main characteristics of the light-emitting device 2 and the comparative light-emitting device 2 at a luminance of about 1000 cd/m². The luminance, CIE chromaticity, and emission spectra were measured at normal temperature with an SR-UL1R spectroradiometer manufactured by TOPCON TECHNOHOUSE CORPORATION.

TABLE 6

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.5 | 0.33 | 8.3 | 0.14 | 0.11 | 11.0 | 11.7 |
| device 2 | | | | | | | |
| Comparative light- | 3.3 | 0.38 | 9.5 | 0.14 | 0.11 | 10.0 | 10.6 |
| emitting device 2 | | | | | | | |

The results in FIG. 37 to FIG. 42 and Table 6 revealed that the light-emitting device 2 fabricated using the low refractive index material of one embodiment of the present invention is an EL device having substantially the same emission spectrum as the comparative light-emitting device 2 and having higher current efficiency and external quantum efficiency than the comparative light-emitting device 2.

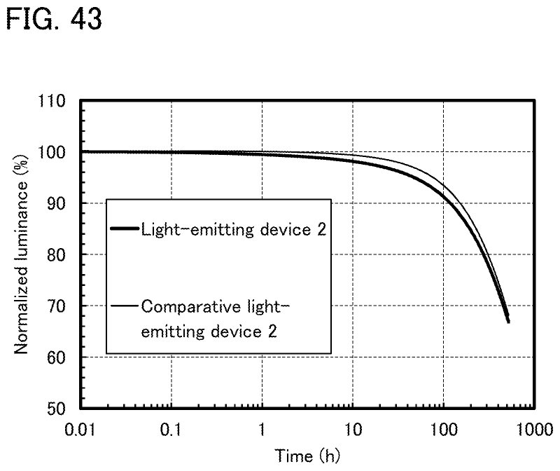

Next, reliability tests were performed on the light-emitting devices. FIG. 43 shows the results of the reliability tests of the light-emitting device 2 and the comparative light-emitting device 2. In FIG. 43, the vertical axis represents normalized luminance (%) with an initial luminance of 100%, and the horizontal axis represents driving time (h) of the devices. As the reliability tests, driving tests at a constant current density of 50 mA/cm² were performed on the light-emitting devices.

The results in FIG. 43 showed that the light-emitting device 2 including the low refractive index material of one embodiment of the present invention had favorable reliability comparable to that of the comparative light-emitting device.

### Example 5

### Synthesis Example 3

In this example, a method for synthesizing 2,4-bis(3′,5′-di-tert-butylbiphenyl-4-yl)-6-[4-(pyrimidin-5-yl)phenyl]pyrimidine (abbreviation: 2,4mmtBuBP-6PmPPm), which is the organic compound represented by Structural Formula (108) in Embodiment 1, is described. The structure of 2,4mmtBuBP-6PmPPm is shown below.

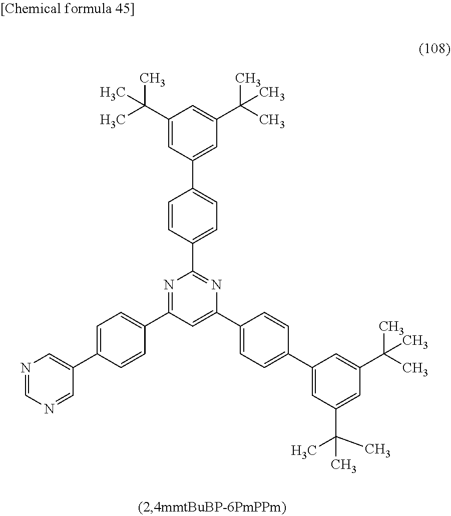 [SMILES: CC(C)(C)c1cc(-c2ccc(-c3cc(-c4ccc(-c5cncnc5)cc4)nc(-c4ccc(-c5cc(C(C)(C)C)cc(C(C)(C)C)c5)cc4)n3)cc2)cc(C(C)(C)C)c1]

<Synthesis of 2,4mmtBuBP-6PmPPm>

Into a three-neck flask were put 0.24 g (1.3 mmol) of 2,4,6-trichloropyrimidine, 0.36 g (1.3 mmol) of 4-(pyrimidin-5-yl)phenyl-4,4,5,5-tetramethyl-1,3,2-dioxaborolane, 6.6 mL of an aqueous solution of potassium carbonate (2 mol/L), and 13 mL of 1,4-dioxane, and the mixture was degassed. Furthermore, 0.046 g (0.066 mmol) of bis(triphenylphosphine)palladium(II) dichloride was added, followed by stirring at 40° C. for 5 hours. To the reacted solution after the stirring, 1.2 g (3.1 mmol) of 2-(3′,5′-di-tert-butylbiphenyl-4-yl)-4,4,5,5-tetramethyl-1,3,2-dioxaborolane and 0.092 g (0.13 mmol) of bis(triphenylphosphine)palladium(II) dichloride were added, followed by stirring at 80° C. for 5 hours. After the stirring, extraction was performed with chloroform and the organic layer was dried with magnesium sulfate. This mixture was gravity-filtered, and the obtained filtrate was concentrated to give a brown solid. This solid was purified by silica gel column chromatography with a developing solvent of toluene and ethyl acetate in a ratio of 10:1 to give a target pale red solid. The synthesis scheme is shown in Formula (c-1).

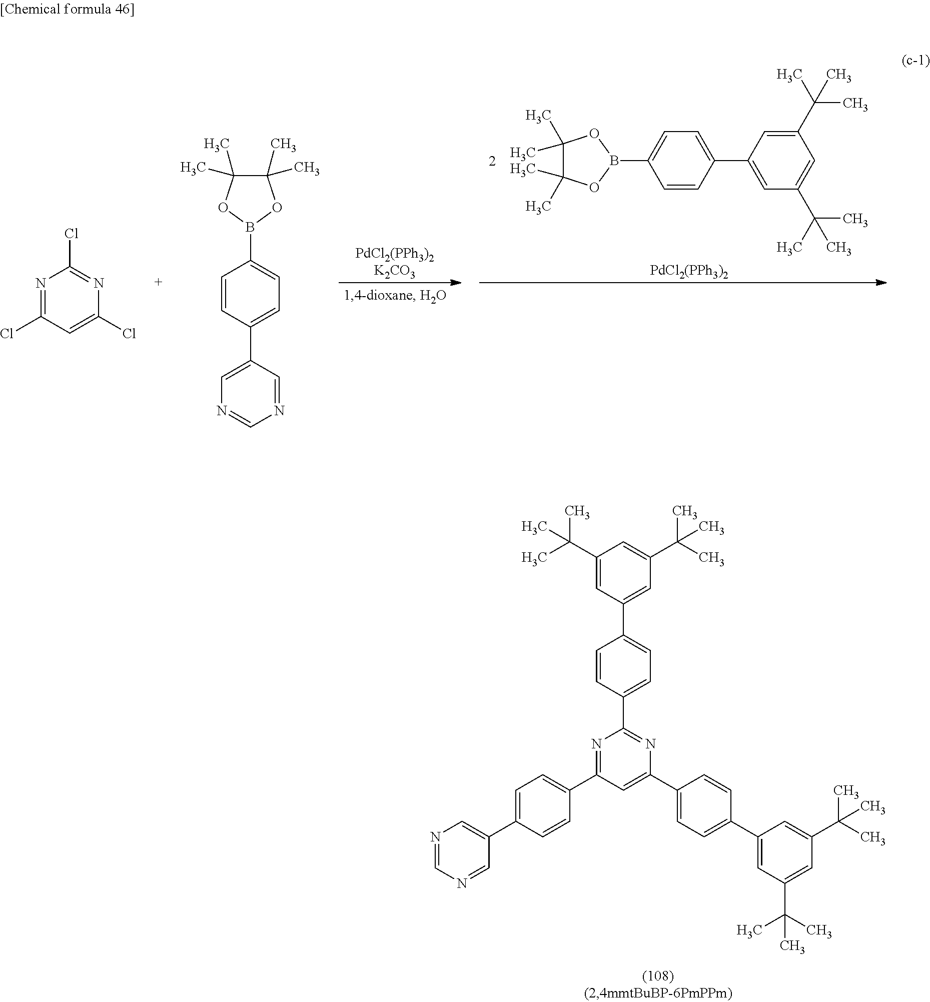

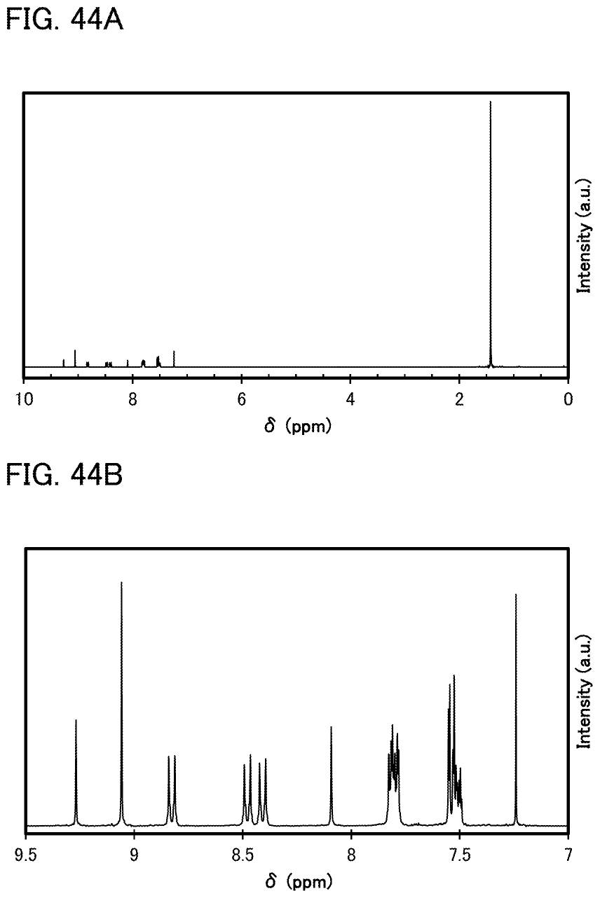

Analysis results by nuclear magnetic resonance (¹H-NMR) spectroscopy of the obtained pale red solid are shown below. FIGS. 44A and 44B show ¹H NMR charts. These results revealed that 2,4mmtBuBP-6PmPPm, which is the organic compound of one embodiment of the present invention represented by Structural Formula (108), was obtained in this example.

¹H NMR (CDCl₃, 300 MHz): δ=1.43 (s, 36H), 7.49-7.55 (m, 6H), 7.78-7.83 (m, 6H), 8.09 (s, 1H), 8.41 (d, J=8.7 Hz, 2H), 8.48 (d, J=8.7 Hz, 2H), 8.83 (d, J=8.4 Hz, 2H), 9.06 (s, 2H), 9.27 (s, 1H).

Next, the molecular weight of the obtained organic compound was measured with a GC/MS detector (ITQ1100 ion trap GC/MS system, manufactured by Thermo Fisher Scientific K.K.). As a result, a main peak with a mass number of 762.46 (mode: EI+) was detected, showing that the target substance 2,4mmtBuBP-6PmPPm was obtained.

### Example 6

### Synthesis Example 4

In this example, a method for synthesizing 4-(3′,5′-di-tert-butylbiphenyl-4-yl)-6-[4-(pyrimidin-5-yl)phenyl]pyrimidine (abbreviation: 4mmtBuBP-6PmPPm), which is the organic compound represented by Structural Formula (199) in Embodiment 1, is described. The structure of 4mmtBuBP-6PmPPm is shown below.

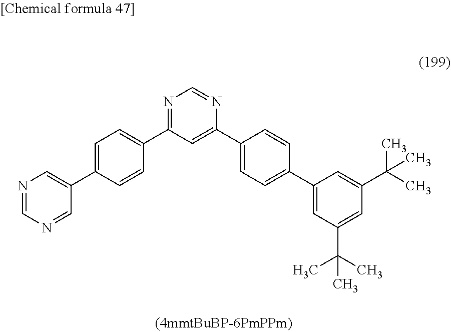 [SMILES: CC(C)(C)c1cc(-c2ccc(-c3cc(-c4ccc(-c5cncnc5)cc4)ncn3)cc2)cc(C(C)(C)C)c1]

<Synthesis of 4mmtBuBP-6PmPPm>

Into a three-neck flask were put 0.48 g (2.0 mmol) of 4,6-dibromopyrimidine, 0.57 g (2.0 mmol) of 4-(pyrimidin-5-yl)phenyl-4,4,5,5-tetramethyl-1,3,2-dioxaborolane, 10 mL of an aqueous solution of potassium carbonate (2 mol/L), and 20 mL of 1,4-dioxane, and the mixture was degassed. Furthermore, 0.071 g (0.10 mmol) of bis(triphenylphosphine)palladium(II) dichloride was added, followed by stirring at 40° C. for 5 hours. To the reacted solution after the stirring, 1.8 g (4.6 mmol) of 2-(3′,5′-di-tert-butylbiphenyl-4-yl)-4,4,5,5-tetramethyl-1,3,2-dioxaborolane and 0.14 g (0.20 mmol) of bis(triphenylphosphine)palladium(II) dichloride were added, followed by stirring at 80° C. for 5 hours. After the stirring, extraction was performed with chloroform and the organic layer was dried with magnesium sulfate. This mixture was gravity-filtered, and the obtained filtrate was concentrated to give a brown solid. This solid was purified by silica gel column chromatography with a developing solvent of toluene and ethyl acetate in a ratio of 10:1 to give a target pale red solid. The synthesis scheme is shown in Formula (d-1).

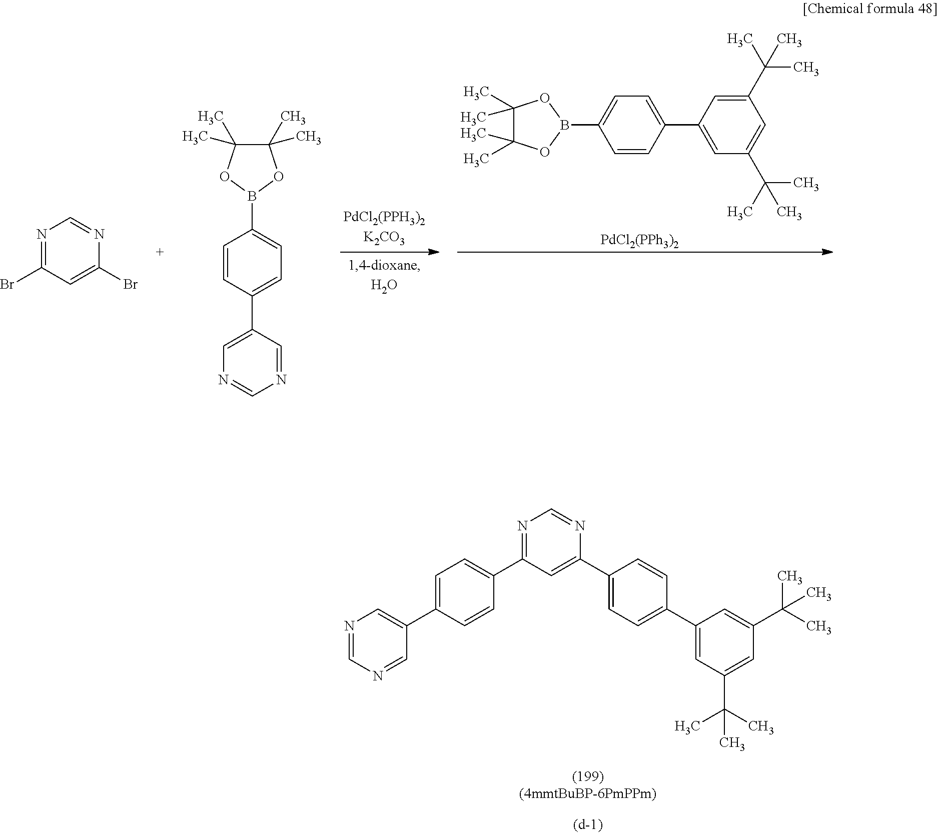

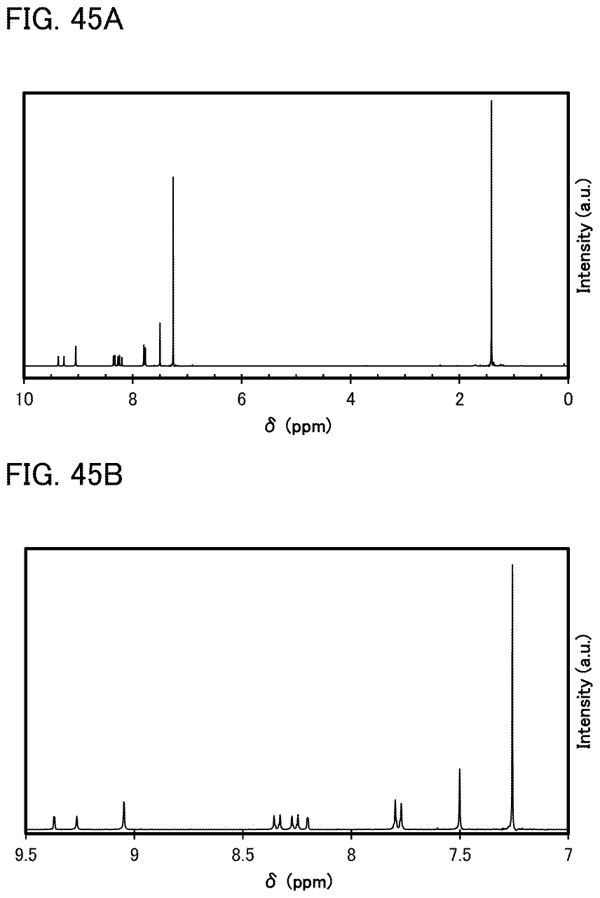

Analysis results by nuclear magnetic resonance (¹H-NMR) spectroscopy of the obtained pale red solid are shown below. FIGS. 45A and 45B show ¹H NMR charts. These results revealed that 4mmtBuBP-6PmPPm, which is the organic compound of one embodiment of the present invention represented by Structural Formula (199), was obtained in this example.

¹H NMR (CDCl₃, 300 MHz): δ=1.41 (s, 18H), 7.50 (s, 3H), 7.78 (d, J=8.4 Hz, 4H), 8.20 (d, J=1.2 Hz, 1H), 8.26 (d, J=8.4 Hz, 2H), 8.34 (d, J=8.4 Hz, 2H), 9.05 (s, 2H), 9.27 (s, 1H), 9.37 (d, J=1.5 Hz, 1H).

Next, the molecular weight of the obtained organic compound was measured with a GC/MS detector (ITQ1100 ion trap GC/MS system, manufactured by Thermo Fisher Scientific K.K.). As a result, a main peak with a mass number of 498.27 (mode: EI+) was detected, showing that the target substance 4mmtBuBP-6PmPPm was obtained.

### Reference Synthesis Example

In this synthesis example, a method for synthesizing 6-methyl-8-quinolinolato-lithium (abbreviation: Li-6mq), the metal complex represented by Structural Formula (vii) and used for some of the light-emitting devices in Examples in this specification, is described. The structural formula of Li-6mq is shown below.

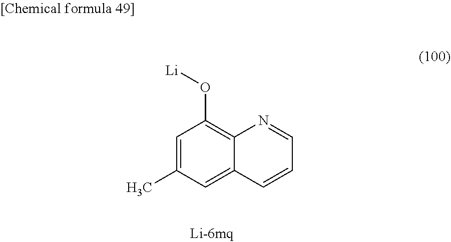

Into a three-neck flask were put 2.0 g (12.6 mmol) of 8-hydroxy-6-methylquinoline and 130 mL of dehydrated tetrahydrofuran (abbreviation: THF), and the mixture was stirred. To this solution was added 10.1 mL (10.1 mmol) of a 1M THF solution of lithium tert-butoxide (abbreviation: tBuOLi), and the mixture was stirred at room temperature for 47 hours. The reaction solution was concentrated to give a yellow solid. Acetonitrile was added to this solid, and the mixture was irradiated with ultrasonic waves and then subjected to filtration to give a pale yellow solid. This washing operation was performed twice. As a residue, 1.6 g of a pale yellow solid of Li-6mq was obtained in a yield of 95%. The synthesis scheme is shown below.

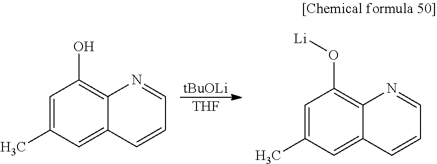

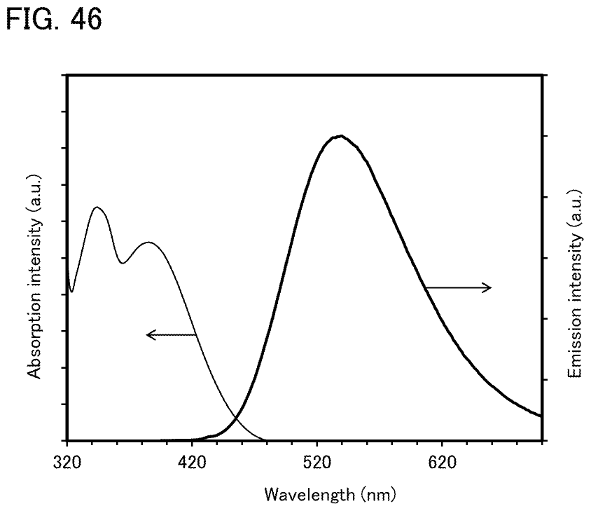

FIG. 46 shows measurement results of an absorption spectrum and an emission spectrum of Li-6mq in dehydrated acetone. The shown absorption spectrum, which was measured with a V550 ultraviolet-visible spectrophotometer manufactured by JASCO Corporation, was obtained by subtracting a measured spectrum of dehydrated acetone alone in a quartz cell from a measured absorption spectrum of the dehydrated acetone solution of Li-6mq in a quartz cell. The emission spectrum was measured with an FP-8600 fluorescence spectrophotometer manufactured by JASCO Corporation.

As shown in FIG. 46, Li-6mq in dehydrated acetone exhibited an absorption peak at 390 nm and an emission wavelength peak at 540 nm (excitation wavelength: 385 nm).

This application is based on Japanese Patent Application Serial No. 2021-011969 filed with Japan Patent Office on Jan. 28, 2021, the entire contents of which are hereby incorporated by reference.

1. An organic compound represented by General Formula (G1): 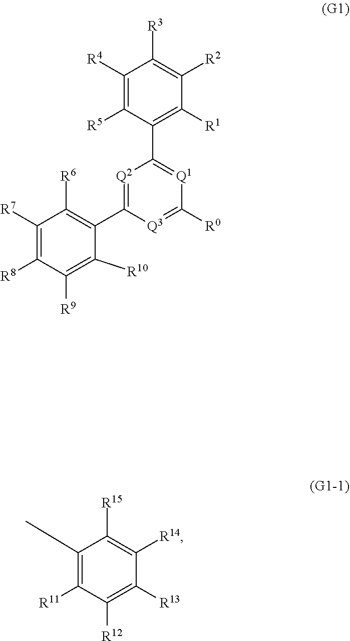
wherein:
one to three of Q¹ to Q³ represent N and when one or two of Q¹ to Q³ represent N, the remaining two or one of Q¹ to Q³ represent CH;
R⁰ represents any of hydrogen, an alkyl group having 1 to 6 carbon atoms, an alicyclic group having 3 to 10 carbon atoms, and a group represented by Formula (G1-1); and
at least one of R¹ to R¹⁵ represents a substituted or unsubstituted group comprising any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group,
wherein when the substituted or unsubstituted group comprising any one of the pyrimidinyl group, the pyrazinyl group, and the triazinyl group comprises one or more substituents, the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group,
wherein the organic compound represented by General Formula (G1) comprises a plurality of hydrocarbon groups each independently selected from an alkyl group having 1 to 6 carbon atoms and an alicyclic group having 3 to 10 carbon atoms, and
wherein the proportion of carbon atoms forming bonds by sp³ hybrid orbitals in the total number of carbon atoms in a molecule of the organic compound is higher than or equal to 10% and lower than or equal to 60%.

2. An organic compound represented by General Formula (G2): 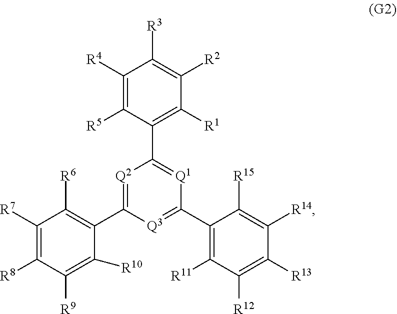
wherein:
one to three of Q¹ to Q³ represent N and when one or two of Q¹ to Q³ represent N, the remaining two or one of Q¹ to Q³ represent CH; and
at least one of R¹ to R¹⁵ represents a substituted or unsubstituted group comprising any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group,
wherein when the substituted or unsubstituted group comprising any one of the pyrimidinyl group, the pyrazinyl group, and the triazinyl group comprises one or more substituents, the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group,
wherein the organic compound represented by General Formula (G2) comprises a plurality of hydrocarbon groups each independently selected from an alkyl group having 1 to 6 carbon atoms and an alicyclic group having 3 to 10 carbon atoms, and
wherein the proportion of carbon atoms forming bonds by sp³ hybrid orbitals in the total number of carbon atoms in a molecule of the organic compound is higher than or equal to 10% and lower than or equal to 60%.

3. An organic compound represented by General Formula (G3): 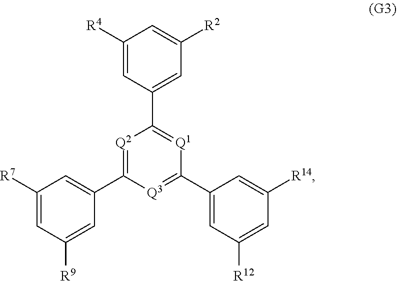
wherein:
one to three of Q¹ to Q³ represent N and when one or two of Q¹ to Q³ represent N, the remaining two or one of Q¹ to Q³ represent CH; and
at least one of R², R⁴, R⁷, R⁹, R¹², and R¹⁴ represents a substituted or unsubstituted group comprising any one of a pyrimidinyl group, a pyrazinyl group, and a triazinyl group, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms forming a ring, and a substituted or unsubstituted pyridinyl group,
wherein when the substituted or unsubstituted group comprising any one of the pyrimidinyl group, the pyrazinyl group, and the triazinyl group comprises one or more substituents, the substituents are each independently any of an alkyl group having 1 to 6 carbon atoms, an alicyclic group having 3 to 10 carbon atoms, an aromatic hydrocarbon group having 6 to 14 carbon atoms forming a ring, and a pyridinyl group,
wherein the organic compound represented by General Formula (G3) comprises a plurality of hydrocarbon groups each independently selected from an alkyl group having 1 to 6 carbon atoms and an alicyclic group having 3 to 10 carbon atoms, and
wherein the proportion of carbon atoms forming bonds by sp³ hybrid orbitals in the total number of carbon atoms in a molecule of the organic compound is higher than or equal to 10% and lower than or equal to 60%.

4. The organic compound according to claim 1,
wherein the substituted or unsubstituted group comprising any one of the pyrimidinyl group, the pyrazinyl group, and the triazinyl group is a group represented by Formula (G1-2): 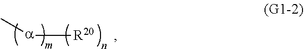
wherein:
α represents a substituted or unsubstituted phenylene group;
R²⁰ represents any one of a substituted or unsubstituted pyrimidinyl group, a substituted or unsubstituted pyrazinyl group, and a substituted or unsubstituted triazinyl group;
m is 0 to 2; and
n is 1 or 2,
wherein when m is 2, a plurality of α's are the same or different from each other, and
wherein when n is 2, a plurality of R²⁰'s are the same or different from each other.

5. The organic compound according to claim 4,
wherein one or both of R² and R⁴ represent the group represented by Formula (G1-2), and
wherein when both R² and R⁴ represent the groups represented by Formula (G1-2), the two groups represented by Formula (G1-2) are the same or different from each other.

6. The organic compound according to claim 1,
wherein the substituted or unsubstituted group comprising any one of the pyrimidinyl group, the pyrazinyl group, and the triazinyl group is a group represented by Formula (G1-3): 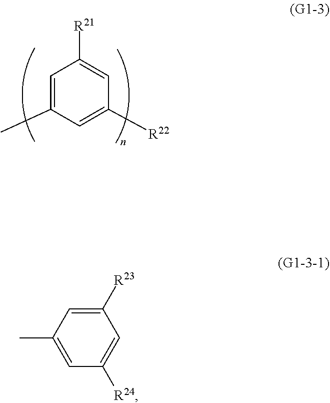
wherein:
R²¹ represents any one of hydrogen, an alkyl group having 1 to 6 carbon atoms, an alicyclic group having 3 to 10 carbon atoms, and a group represented by Formula (G1-3-1);
R²² represents the group represented by Formula (G1-3-1);
n is 0 to 2;
R²³ and R²⁴ each independently represent any one of hydrogen, an alkyl group having 1 to 6 carbon atoms, an alicyclic group having 3 to 10 carbon atoms, a substituted or unsubstituted pyrimidinyl group, a substituted or unsubstituted pyrazinyl group, and a substituted or unsubstituted triazinyl group; and
at least one of R²³ and R²⁴ represents any one of a substituted or unsubstituted pyrimidinyl group, a substituted or unsubstituted pyrazinyl group, and a substituted or unsubstituted triazinyl group, and
wherein when n is 2, a plurality of R²¹'s are the same or different from each other.

7. The organic compound according to claim 6,
wherein one or both of R² and R⁴ represent the group represented by Formula (G1-3), and
wherein when both R² and R⁴ represent the groups represented by Formula (G1-3), the two groups represented by Formula (G1-3) are the same or different from each other.

8. The organic compound according to claim 1,
wherein when the aromatic hydrocarbon group having 6 to 14 carbon atoms forming a ring comprises a substituent, the substituent is any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group which has 6 to 14 carbon atoms forming a ring and to which an alkyl group having 1 to 6 carbon atoms or an alicyclic group having 3 to 10 carbon atoms is bonded.

9. The organic compound according to claim 1,
wherein the aromatic hydrocarbon group having 6 to 14 carbon atoms forming a ring is any one of a phenyl group, a naphthyl group, a phenanthrenyl group, and a fluorenyl group.

10. The organic compound according to claim 1,
wherein the aromatic hydrocarbon group having 6 to 14 carbon atoms forming a ring is represented by any one of Formulae (ra-1) to (ra-16):

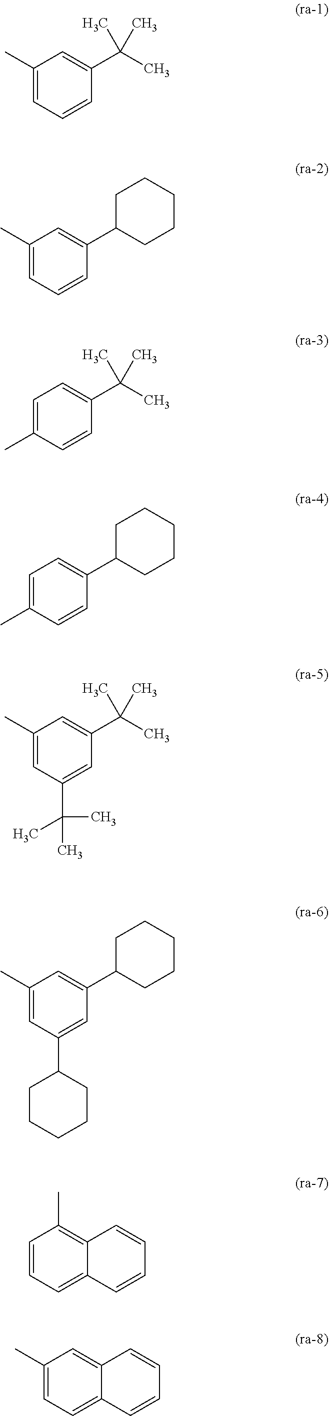

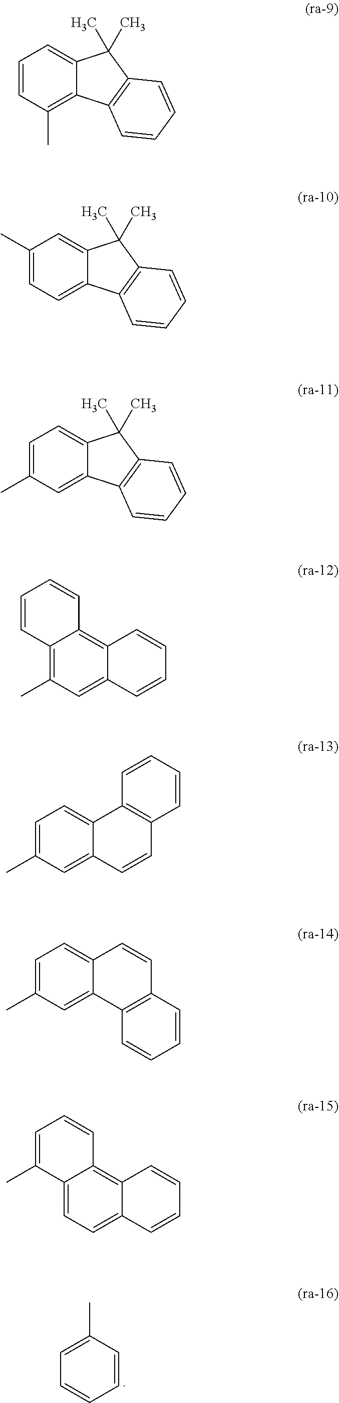

11. The organic compound according to claim 1,
wherein the substituted or unsubstituted pyridinyl group is an unsubstituted pyridinyl group or a pyridinyl group to which one or more methyl groups are bonded.

12. The organic compound according to claim 1,
wherein the alicyclic group is a cycloalkyl group having 3 to 6 carbon atoms.

13. The organic compound according to claim 1,
wherein the alkyl group having 1 to 6 carbon atoms is a branched alkyl group having 3 to 5 carbon atoms.

14. The organic compound according to claim 3,
wherein:
R² represents a group represented by Formula (R²-1);
R⁴, R⁷, R⁹, R¹², and R¹⁴ each independently represent any one of groups represented by Formulae (r-1) to (r-44);
β represents a substituted or unsubstituted phenylene group or a substituted or unsubstituted biphenyldiyl group;
R²⁵ represents any one of the groups represented by Formulae (r-1) to (r-24); and
n is 1 or 2,
wherein the organic compound represented by General Formula (G3) comprises a plurality of hydrocarbon groups each independently selected from an alkyl group having 1 to 6 carbon atoms and an alicyclic group having 3 to 10 carbon atoms, and
wherein the proportion of carbon atoms forming bonds by sp³ hybrid orbitals in the total number of carbon atoms in a molecule of the organic compound is higher than or equal to 10% and lower than or equal to 60%,

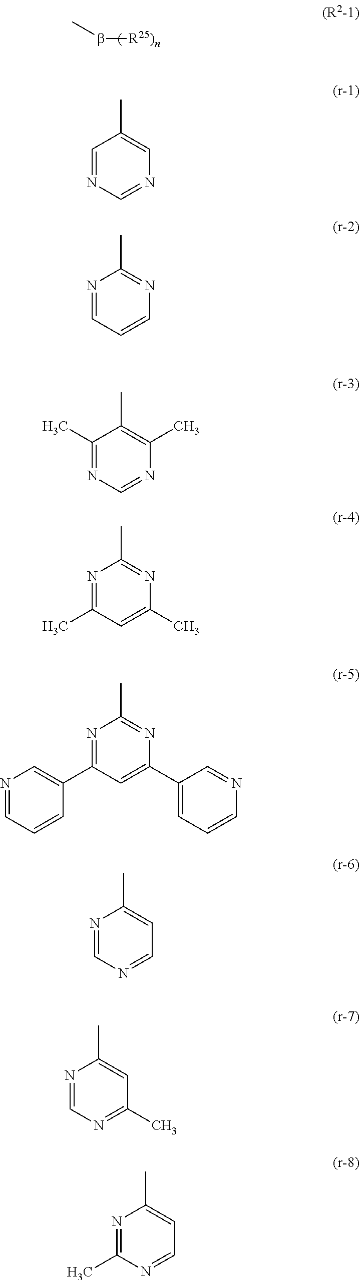

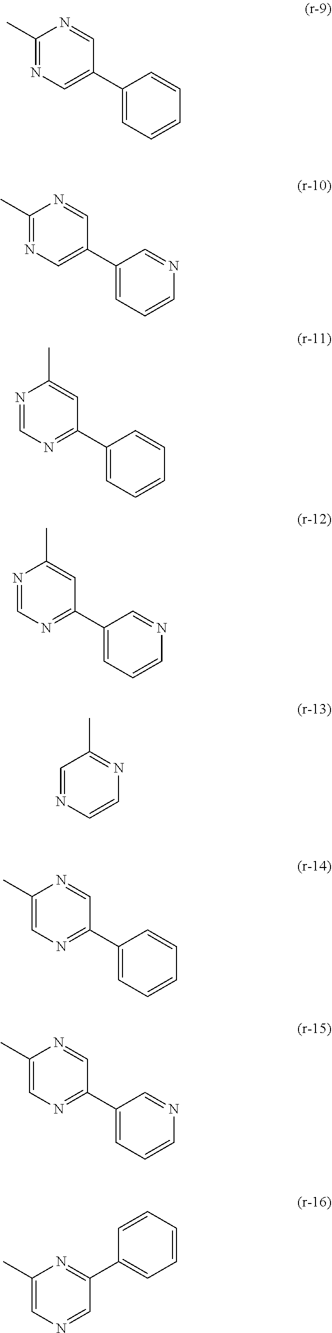

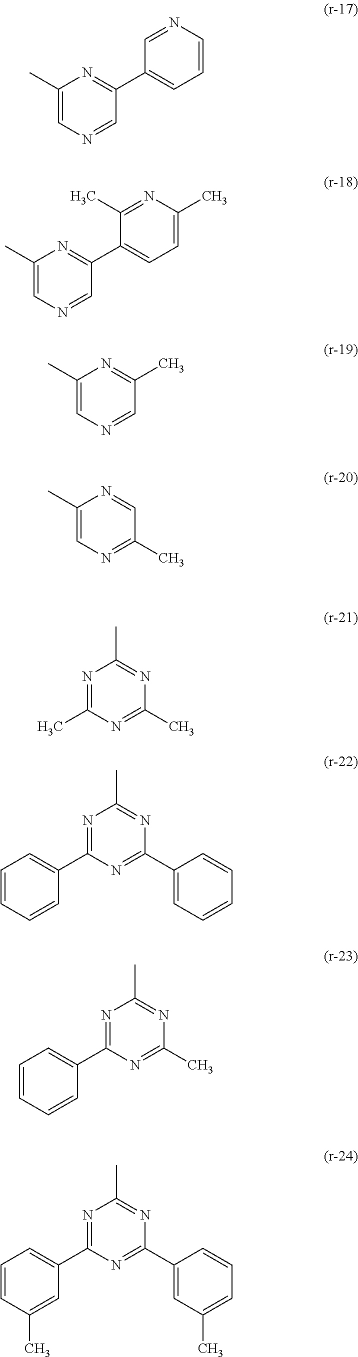

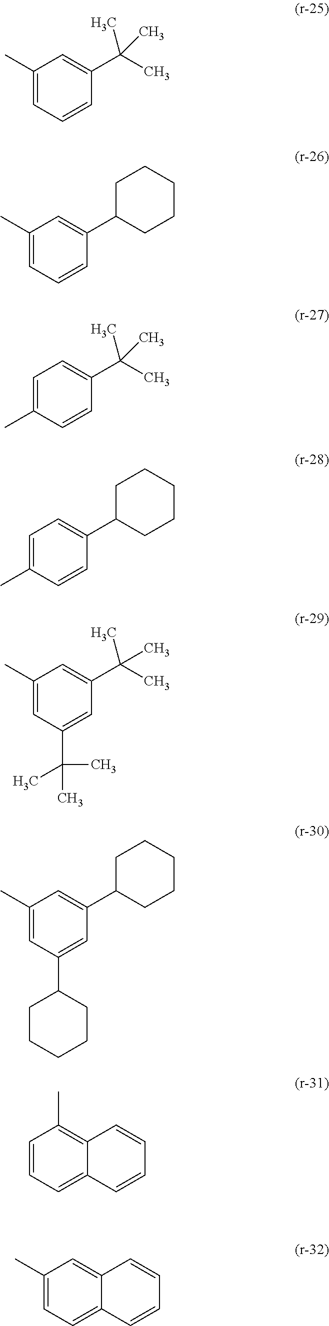

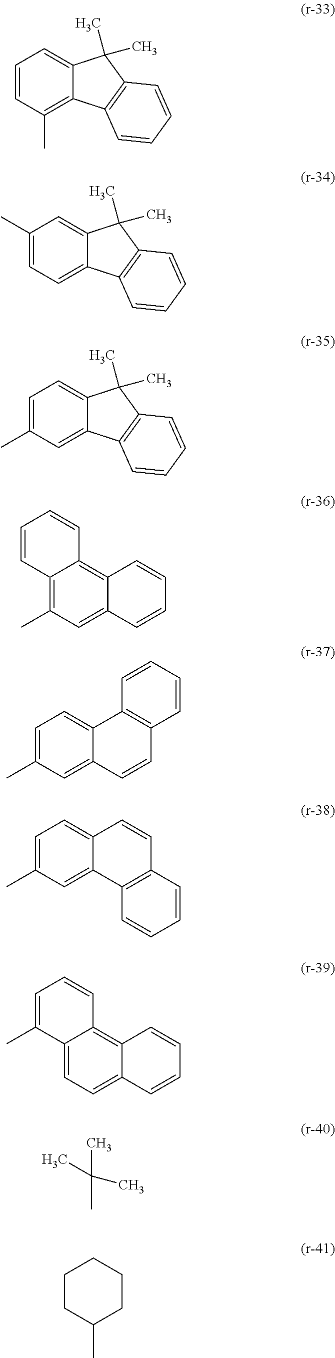

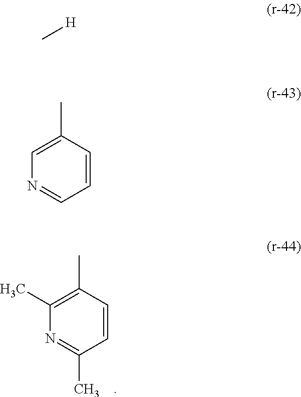

15. The organic compound according to claim 14,
wherein β represents a group represented by any one of Formulae (β-1) to (β-14),

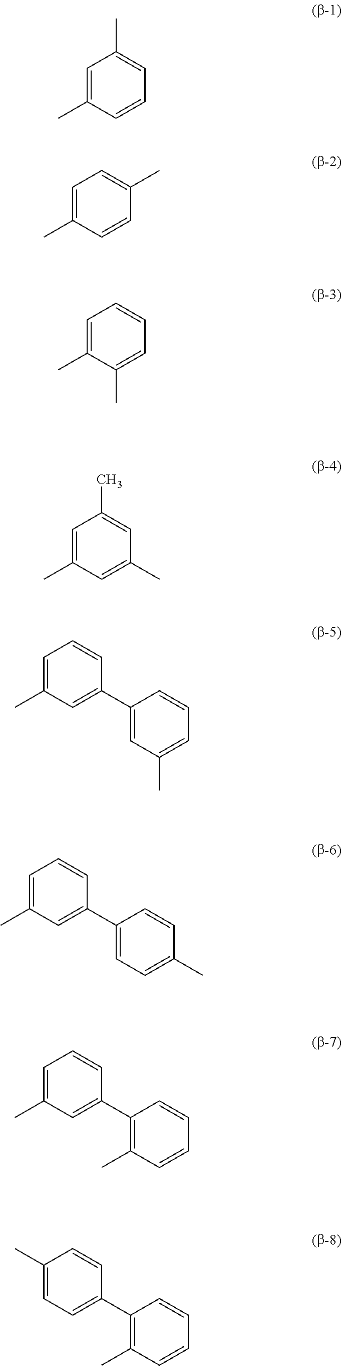

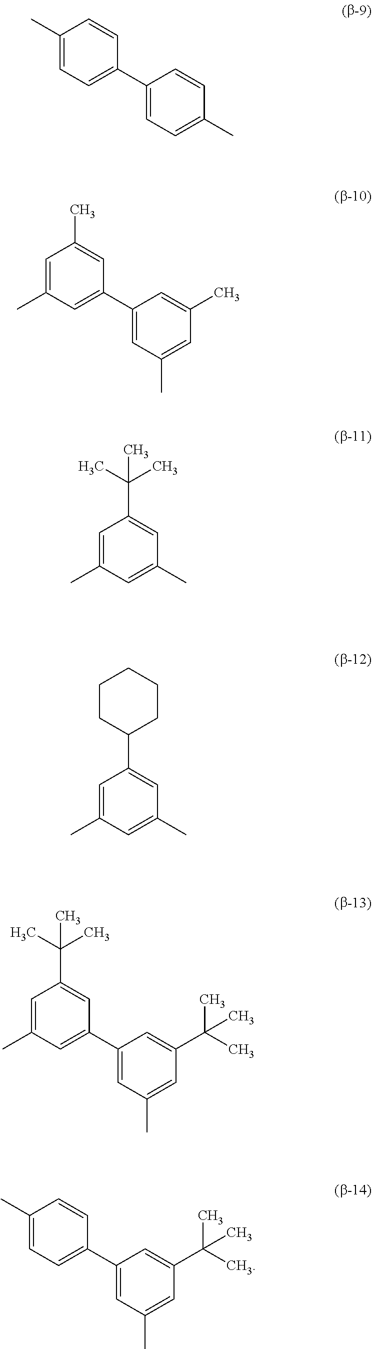

16. The organic compound according to claim 1, wherein the organic compound is represented by Structural Formula (137) or (154),

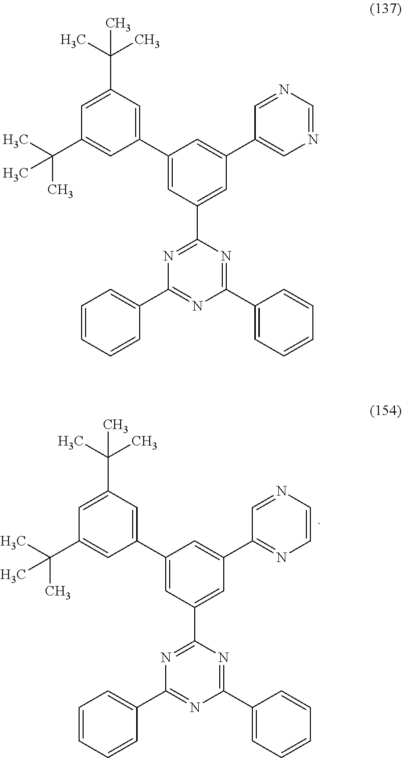 [SMILES: CC(C)(C)c1cc(-c2cc(-c3cncnc3)cc(-c3nc(-c4ccccc4)nc(-c4ccccc4)n3)c2)cc(C(C)(C)C)c1 ; CC(C)(C)c1cc(-c2cc(-c3cnccn3)cc(-c3nc(-c4ccccc4)nc(-c4ccccc4)n3)c2)cc(C(C)(C)C)c1]

17. A light-emitting device comprising the organic compound according to claim 1.

18. An electronic appliance comprising:
the light-emitting device according to claim 17; and
a sensor unit, an input unit, or a communication unit.

19. A light-emitting apparatus comprising:
the light-emitting device according to claim 17; and
a transistor or a substrate.

20. A lighting device comprising:
the light-emitting device according to claim 17; and
a housing.

21. The organic compound according to claim 2,
wherein the substituted or unsubstituted group comprising any one of the pyrimidinyl group, the pyrazinyl group, and the triazinyl group is a group represented by Formula (G1-2): 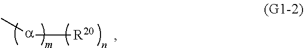
wherein:
α represents a substituted or unsubstituted phenylene group;
R²⁰ represents any one of a substituted or unsubstituted pyrimidinyl group, a substituted or unsubstituted pyrazinyl group, and a substituted or unsubstituted triazinyl group;
m is 0 to 2; and
n is 1 or 2,
wherein when m is 2, a plurality of α's are the same or different from each other, and
wherein when n is 2, a plurality of R²⁰'s are the same or different from each other.

22. The organic compound according to claim 21,
wherein one or both of R² and R⁴ represent the group represented by Formula (G1-2), and
wherein when both R² and R⁴ represent the groups represented by Formula (G1-2), the two groups represented by Formula (G1-2) are the same or different from each other.

23. The organic compound according to claim 3,
wherein the substituted or unsubstituted group comprising any one of the pyrimidinyl group, the pyrazinyl group, and the triazinyl group is a group represented by Formula (G1-2): 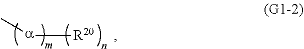
wherein:
α represents a substituted or unsubstituted phenylene group;
R²⁰ represents any one of a substituted or unsubstituted pyrimidinyl group, a substituted or unsubstituted pyrazinyl group, and a substituted or unsubstituted triazinyl group;
m is 0 to 2; and
n is 1 or 2,
wherein when m is 2, a plurality of α's are the same or different from each other, and
wherein when n is 2, a plurality of R²⁰'s are the same or different from each other.

24. The organic compound according to claim 23,
wherein one or both of R² and R⁴ represent the group represented by Formula (G1-2), and
wherein when both R² and R⁴ represent the groups represented by Formula (G1-2), the two groups represented by Formula (G1-2) are the same or different from each other.
